(19) United States
(12) Patent Application Publication
(10) Pub. No.: US 2011/0195056 A1
(43) Pub. Date: Aug. 11, 2011

(54) COMPOSITIONS COMPRISING HEPATOCYTE-LIKE CELLS AND USES THEREOF

(75) Inventors: Howard I. Pryor, Washington, DC (US); Joseph P. Vacanti, Boston, MA (US); David H. Lum, Arlington, MA (US); Tim D. Ahfeldt, Cambridge, MA (US); Chad Cowan, Boston, MA (US)

(73) Assignee: The General Hospital Corporation, Boston, MA (US)

(21) Appl. No.: 12/999,238

(22) PCT Filed: Jul. 31, 2009
(86) PCT No.: PCT/US09/52482

§ 371 Date: Apr. 29, 2011

Related U.S. Application Data
(62) Division of application No. 61/137,479, filed on Jul. 31, 2008.
Application No. 61/137,479 is a division of application No. 61/100,946, filed on Sep. 29, 2008.

(51) Int. Cl. A61K 35/407; C12N 15/00; C12N 5/07; C12Q 1/02; C12P 21/00; A61P 1/16

(52) U.S. Cl. 424/93.21; 435/455; 435/325; 435/29; 435/69.1

(57) ABSTRACT

The invention generally features methods for generating hepatocytes from a variety of pluripotent stem cells, including adipose mesenchymal stem cells, therapeutic compositions featuring such cells, and methods of using them for the treatment of subjects.

### CROSS-REFERENCE TO RELATED APPLICATION

This application claims the benefit of the U.S. Provisional Application Nos. 61/137,479, filed Jul. 31, 2008, and 61/100,946, filed Sep. 29, 2008, the entire contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant Nos: Nos. T32DK07754-09. The government has certain rights in the invention.

### BACKGROUND OF THE INVENTION

The liver is the largest internal organ and the largest gland in the human body. The adult human liver normally weighs between 1.4-1.6 kilograms (3.1-3.5 pounds), and it is a soft, pinkish-brown, triangular four-lobed organ that lies on the right of the stomach and overlies the gallbladder. It is located on the right side of the upper abdomen below the diaphragm anatomy. Two lobes of the liver are visible from the front (anterior). The falciform ligament, visible on the anterior side of the liver, divides the liver into a left anatomical lobe, and a right anatomical lobe. When the liver flipped over, looking at it from behind (the visceral surface), there are two additional lobes between the right and left. Each of the lobes is made up of lobules, a vein goes from the centre of each lobule which then joins to the hepatic vein to carry blood out from the liver. On the surface of the lobules there are ducts, veins and arteries that carry fluids to and from them.

The liver is necessary for survival; a human can only last up to 24 hours without liver function. Overall, the liver performs over 500 jobs, and produces over 1,000 essential enzymes. The various functions of the liver are carried out by the liver cells or hepatocytes.

The liver plays a major role in carbohydrate, protein, and lipid metabolism. The liver is responsible for gluconeogenesis (the synthesis of glucose from certain amino acids, lactate or glycerol), glycogenolysis (the breakdown of glycogen into glucose), glycogenesis (the formation of glycogen from glucose) the breakdown of insulin and other hormone, convert lactic acid to alanine (the mainstay of protein metabolism), cholesterol synthesis and lipogenesis, the production of triglycerides (fats). The liver has blood vessels connecting it to the spleen, pancreas, stomach, small intestine, and large intestine, so it can process the nutrients and by-products of food digestion.

In addition, the liver has a number of functions in connection with regulating a wide variety of high-volume biochemical reactions requiring very specialized tissues in the body. These include glycogen storage, decomposition of red blood cells, plasma protein synthesis, and detoxification. It produces and excretes bile (a greenish liquid) required for emulsifying fats. Some of the bile drains directly into the duodenum, and some is stored in the gallbladder. It produces coagulation factors I (fibrinogen), II (prothrombin), V, VII, IX, X and XI, as well as protein C, protein S and antithrombin. The liver breaks down haemoglobin, creating metabolites that are added to bile as pigment (bilirubin and biliverdin). The liver breaks down toxic substances and most medicinal products in a process called drug metabolism. This sometimes results in toxication, when the metabolite is more toxic than its precursor. The liver converts ammonia to urea. The liver stores a multitude of substances, including glucose (in the form of glycogen), vitamin B12, iron, and copper. In the first trimester fetus, the liver is the main site of red blood cell production. By the 32nd week of gestation, the bone marrow has almost completely taken over that task. The liver is responsible for immunological effects—the reticuloendothelial system of the liver contains many immunologically active cells, acting as a ‘sieve’ for antigens carried to it via the portal system. The liver produces albumin, the major osmolar component of blood serum.

Because the liver is so vital to the physiological homeostasis in the body, the liver is among the few internal human organs capable of natural regeneration of lost tissue; as little as 25% of a liver can regenerate into a whole liver. This is predominantly due to the hepatocytes re-entering the cell cycle (i.e. the hepatocytes go from the quiescent G0 phase to the G1 phase and undergo mitosis). During liver regeneration after partial hepatectomy, normally quiescent hepatocytes undergo one or two rounds of replication to restore the liver mass by a process of compensatory hyperplasia. In contrast to the regenerative process after partial hepatectomy, which is driven by the replication of existing hepatocytes, liver repopulation after acute liver failure depends on the differentiation of progenitor cells. Such cells are also present in chronic liver diseases.

When there is sudden, massive destruction of liver cells or by insults that severely inhibit the ability of hepatocytes to accomplish their normal function, liver failure occurs. Fibrous tissue form of in the liver, replacing the dead liver cells. This is known as cirrhosis of the liver. The most common causes are various liver diseases: hepatitis B virus, non-A, non-B, non-C viral hepatitis; alcoholism; certain hereditary diseases: Wilson's disease and haemochromatosis; and exposure to certain drugs and toxins. Acetaminophen ingestion is responsible for 10% of acute liver failure cases in the United States. In 2005, 27,530 deaths in America was attributed to chronic liver disease and cirrhosis (National vital statistics reports by the Centers for Disease Control and Prevention, vol. 56, No. 10, Apr. 24, 2008), and every year many more are diagnosed with acute or chronic liver disease and cirrhosis. Long-term treatment options include liver transplantation and liver dialysis. However, there is always a shortage of livers suitable for transplantation. Currently, there is no artificial organ or device capable of emulating all the functions of the liver, although some functions can be emulated by liver dialysis.

A bioartificial liver device (BAL) is an artificial extracorporeal supportive device for an individual who is suffering from acute liver failure. BALs are essentially bioreactors, with embedded hepatocytes (liver cells) that perform the functions of a normal liver. A series of studies in 2004 have shown that a BAL device reduced mortality by about half in acute liver failure cases. The studies, which covered 171 patients in the U.S. and Europe, compared standard supportive care to the use of a bioreactor device using pig liver cells. Currently most BALs use porcine hepatocyte because of lack of a supply of human hepatocytes. Isolated primary hepatocytes from human cadaver liver have exhibited a limited replicating lifespan in culture. In addition, when stimulated to divide in culture they have generally lost differentiated functions such as the ability to synthesize and secrete albumin and transferrin. Hence, there is a significant effort to develop a renewable source of human hepatocytes for this purpose. In addition, the renewable source of human hepatocytes can provide cultured hepatocytes for other applications such as gene therapy, cell transplants, drug production, clinical and academic research of the mechanisms of liver regeneration and differentiation, and drug and chemical testing.

Long-term treatment options include liver transplantation and liver dialysis. However, there is always a shortage of livers suitable for transplantation. Currently, there is no artificial organ or device capable of emulating all the functions of the liver, although some functions can be emulated by liver dialysis.

A renewable source of human hepatocytes are urgently required not only for therapeutic use, but also for drug screening and in vitro research use.

### SUMMARY OF THE INVENTION

As described below, the present invention features methods for generating hepatocytes from a variety of cell types, including adipose mesenchymal stem cells, as well as therapeutic compositions and methods featuring the use of such cells.

In one aspect, the invention provides a method for generating a hepatocyte-like cell, the method involving culturing a stem cell in a three dimensional culture; expressing in the stem cell a heterologous nucleic acid molecule encoding a HNF-4-α polypeptide, contacting the stem cell with one or more agents selected from the group consisting of epidermal growth factor, basic fibroblast growth factor, hepatocyte growth factor, nicotinamide, and oncostatin M, thereby generating a hepatocyte-like cell.

In another aspect, the invention provides a method for generating a hepatocyte-like cell, the method involving culturing a adipose-derived mesenchymal stem cell (ADMSC) in a three dimensional culture; expressing in the stem cell a heterologous nucleic acid molecule encoding a HNF-4-α polypeptide; contacting the stem cell with epidermal growth factor, basic fibroblast growth factor, hepatocyte growth factor, nicotinamide, and oncostatin M, thereby generating a hepatocyte-like cell.

In another aspect, the invention features a hepatocyte-like cell produced by the method of a previous aspect or any other method delineated herein.

In yet another aspect, the invention provides a hepatocyte-like cell comprising a heterologous nucleic acid molecule encoding a HNF-4-α polypeptide and expressing a hepatocyte marker or hepatocyte biological activity.

In yet another aspect, the invention provides a pharmaceutical composition comprising a hepatocyte-like cell comprising a heterologous nucleic acid molecule encoding a HNF-4-α polypeptide and expressing a hepatocyte marker or hepatocyte biological activity.

In yet another aspect, the invention provides a method for treating a subject in need of an increase in liver function, the method comprising administering to the subject a hepatocyte-like cell of any previous aspect or otherwise delineated herein, thereby increasing liver function.

In still another aspect, the invention provides a method of characterizing agent toxicity, the method comprising contacting a hepatocyte-like cell produced by the method of any previous aspect with an agent and identifying a reduction in the cell's metabolic activity or viability.

In still another aspect, the invention provides a method for producing a coagulation factor, the method comprising culturing a hepatocyte-like cell produced by the method of any previous aspect, and isolating from the cell a coagulation factor.

In still another aspect, the invention provides a method of neutralizing a toxic compound in a bodily fluid of a mammal, the method comprising contacting the fluid with a hepatocyte-like cell produced by the method of any previous aspect, wherein the contacting step takes place in a perfusion device.

In still another aspect, the invention provides a bioartificial liver device comprising a hepatocyte-like cell produced by the method of any of claims 1-8.

In still another aspect, the invention provides a method of treating a subject for reduced liver function, the method comprising administering to the subject the bioartificial liver device of the previous aspect or any bioartificial liver device described herein.

In various embodiments of any of the above aspects, the stem cell is an adipose-derived mesenchymal stem cell (ADMSC), embryonic stem cell, mesenchymal stem cell, tissue-specific stem cell, or induced pluripotent stem cell. In other embodiments, the stem cell further contains a heterologous nucleic acid molecule that is any one or more of HNF-3β, HNF-1α, and CEBP/α. In still other embodiments, the HNF-4-α polypeptide is expressed in a viral vector. In still other embodiments, the hepatocyte-like cell expresses one or more hepatocyte markers that is any one or more of glucose-6-phosphatase, albumin secretion, arginase Type I, CYP 3A4, and bile or exhibits a biological activity that is any one or more of glucose metabolism, protein synthesis, urea production, xenobiotic detoxification, and biliary secretion. In still other embodiments, the cell expresses HNF-4-α, Cyp-3A4, and Zona Occludens-1. In still other embodiments, the three dimensional culture is a collagen sandwich culture.

Embodiments of the invention are based on the discovery that non-committed, non-differentiated stem cells can be induced to differentiated into hepatocytes by (1) transducing and over-expression of exogenous copies of master hepatocyte transcription factors in the stem cells, and (2) treating the transduced stem cells with a course of growth factors and differentiation factors. The hepatocyte transcription factors are hepatocyte nuclear factor 4 alpha (HNF-4-α), hepatocyte nuclear factor 1alpha (HNF-1α), hepatocyte nuclear factor 3beta (HNF-3β), and CCAAT/enhancer binding protein beta (CEBP beta). Various combinations of these hepatocyte transcription factors can be transduced into stem cells. The growth factors and differentiation factors are epithelial growth factor (EGF), basic fibroblast growth factor (bFGF), hepatocyte growth factor (HGF), nicotinamide, and oncostatin M (OSM). The differentiated ADMSCs are hepatocytes-like cells exhibiting the expression of many hepatocyte specific proteins such as albumin, cytochrome P450, and arginase; and by-products such as urea and bile.

Accordingly, the present invention provides herein is a method for producing hepatocyte-like cells from stem cells, the method comprising: (a) introducing an exogenous copy of at least one gene selected from the group consisting of hepatic nuclear factor-4-alpha (HNF-4-α), hepatic nuclear factor-3 (HNF-3β), hepatic nuclear factor-1 (HNF-1α) and CCAAT/enhancer binding protein beta (CEBP beta), into a stem cell; (b) ex-vivo culturing the stem cell of step (a) in a culture medium comprising epidermal growth factor (EGF) and basic fibroblast growth factor (bFGF) for 1-7 days; (c) ex-vivo culturing the stem cell of step (b) in a culture medium comprising hepatocyte growth factor (HGF), basic fibroblast growth factor (bFGF) and nicotinamide for 1-7 days; and (d) ex-vivo culturing the stem cell of step (c) in a culture medium comprising oncostatin M (OSM) for 1-180 days; wherein the method produces a hepatocyte-like cell, wherein the hepatocyte-like cell has at least three of the following characteristics: evidence of glycogen storage; antibody-detectable expression of albumin; absence of antibody-detectable expression of α-fetoprotein; evidence of cytochrome p450 (CYP 3A4) activity; evidence of glucose-6-phosphatase activity; evidence of arginase Type I of the urea cycle; or evidence of bile secretion.

In one embodiment, the transduced stem cells are embedded in an extracellular matrix, such as collagen.

In one embodiment, the stem cell is pluripotent. In another embodiment, the stem cell is multi-potent. In other embodiments, the stem cell is an adult, fetal or embryonic stem cell.

In some embodiments, the stem cell is isolated from umbilical, placenta, amniotic fluid, chorion villi, blastocysts, bone marrow, adipose tissue, brain, peripheral blood, cord blood, menstrual blood, blood vessels, skeletal muscle, skin and liver. In some embodiments, the stem cell is reprogrammed differentiated cells.

In one embodiment, provided herein is a tissue engineered liver comprising a hepatocyte-like cell produced by the methods set forth herein.

In one embodiment, provided herein is a hepatocyte-based bio-artificial liver (BAL) comprising a hepatocyte-like cell produced by the methods described herein.

In one embodiment, provided herein is a use of a hepatocyte-like cell produced by the methods described herein in the production of coagulation factors.

In one embodiment, provided herein is a method of evaluating the toxicity of a compound in vitro, comprising (a) providing a hepatocyte-like cell described herein; (b) contacting the hepatocyte-like cell with the compound to generate a cell supernatant; (c) measuring the metabolic activity or viability of the hepatocyte-like cell, wherein a decrease in metabolic activity or viability in the presence of the supernatant compared to that in the absence of the supernatant indicates that the compound is toxic in vivo.

In one embodiment, provided herein is a method of evaluating the toxicity of a compound in vitro, comprising (a) providing a first hepatocyte-like cell described herein; (b) contacting the first hepatocyte-like cell with the compound to generate a cell supernatant; (c) removing the cell supernatant from the first hepatocyte-like cell; (d) providing a second hepatocyte-like cell described herein; (e) contacting the second hepatocyte-like cell of step (d) with the supernatant; and (f) measuring the metabolic activity or viability of the second hepatocyte-like cell, wherein a decrease in metabolic activity or viability in the presence of the supernatant compared to that in the absence of the supernatant indicates that the compound is toxic in vivo. In one embodiment, provided herein is a method of neutralizing a toxic compound in a bodily fluid of a mammal, comprising contacting the fluid with a hepatocyte-like cell described herein, wherein the contacting step takes place in a perfusion device. In one embodiment, the transduced stem cell is embedded in a collagen sandwich. In one embodiment, the culture medium for differentiating the transduced stem cell further comprises KO-Serum replacement and plasmanate.

The invention provides methods for generating hepatocytes from stem cells. Compositions and articles defined by the invention were isolated or otherwise manufactured in connection with the examples provided below. Other features and advantages of the invention will be apparent from the detailed description, and from the claims.

### BRIEF DESCRIPTION OF THE DRAWINGS

FIG. 1 is a micrograph showing nuclear co-localization of HNF4-α and DAPI

(Blue—DAPI; Red—HNF4-α; Green—GFP) in transduced adipose-derived mesenchymal stem cells (MSC).

FIG. 2 is a micrograph showing that HNF4α induced expression of endogenous cytochrome p450 3A4 (CYP-3A4) in transduced adipose-derived mesenchymal stem cells (MSC).

FIG. 3 is a micrograph showing staining for the Zona Occludens-1, DAPI, and Combined (Red—CYP 3A4; Blue—DAPI).

FIG. 4 is a micrograph showing Hepatocyte Nuclear Factor-4-α (HNF-4-α) staining in HepG2 cells.

FIG. 5 is a micrograph showing CYP-3A4 and DAPI staining in HepG2 cells.

FIGS. 6A and 6B are micrographs showing CYP-3A4 and DAPI staining in primary hepatocytes. FIG. 6A shows staining in a cytospin of hepatocytes. FIG. 6B shows CYP-3A4 and DAPI staining in primary hepatocytes.

FIG. 7 is a micrograph showing HNF4α (top) and DAPI (bottom) staining in primary human hepatocytes.

FIG. 8 provides two micrograph showing HNF-4-α staining in transfected 3T3 Murine Fibroblasts.

FIG. 9 is a schematic diagram illustrating the Invitrogen Gateway cloning system.

FIG. 10 is a schematic diagram depicting the donor pDONR221 plasmid.

FIG. 11 is a schematic diagram depicting the pCC#DL38 (pFUdGWtetOP/rfa verB) DEST (destination) plasmid.

FIG. 12 is a schematic diagram depicting the pCC#DL71 (pFUdGWtetOP/HNF4□V2) carrying the HNF4αgene.

FIG. 13 is a schematic diagram depicting the pCC#DL68 (pFUdGWtetOP/C/EBPα) carrying the C/EBP alpha gene.

FIG. 14 is a schematic diagram depicting the pCC#DL94 (pFUdGWtetOP/HNF1α) carrying the HNF1αgene.

FIG. 15 is a schematic diagram depicting the pCC#DL96 (pFUdGWtetOP/FOXA2) carrying the HNF31β gene.

FIG. 16 shows the sequence and restriction map of HNF4α gene.

FIG. 17 shows the sequence and restriction map of HNF1α gene.

FIG. 18 shows the sequence and restriction map of (FOXA2) HNF31β gene.

### DETAILED DESCRIPTION OF THE INVENTION

As described below, the present invention features methods for generating hepatocytes from a pluripotent, normally proliferative cell population (e.g., embryonic stem cells; mesenchymal stem cells; tissue-specific stem cells; or induced pluripotent stem cells). This process involves the transfer of plasmids containing four genes that function in the maintenance of a mature hepatocyte: HNF4-α; HNF1-α; HNF3-β (FOXA2); and CEBP/α, either through transfection or transduction into the pluripotent cell source. The present invention is based, at least in part, on the discovery that adipose-derived mesenchymal stem cells (ADMSCs) could be induced to differentiate into hepatocytes by over-expressing a nuclear transcription factor essential to the hepatocyte genotype: HNF4-α, which induced expression of the liver-specific enzyme CYP 3A4 and tight junction formation as indicated by staining for Zona Occludens-1.

By inducing the expression of a combination of hepatocyte nuclear transcription factors and the application of extra-cellular signals, a population of pluripotent, normally proliferative cells can be re-programmed to perform the functions of mature human hepatocytes. These re-programmed cells can then be used for a variety of tissue engineering applications including: high-throughput drug screening, coagulation factor synthesis, hepatic dialysis, and eventually organ support and replacement therapy.

### DEFINITIONS OF TERMS

As used herein, the term “stem cell” refers to an unspecialized cell that gives rise to a specific specialized cell, such as a blood. A “stem cell” has the ability to self-renewal, i.e. to go through numerous cycles of cell division while maintaining the undifferentiated state, and has potency, i.e. the capacity to differentiate into specialized cell types.

As used herein, the term “pluripotent stem cell” refers to a stem cell having the potential to make any differentiated cell in the body but not those of the placenta which is derived from the trophoblast.

As used herein, the term “multipotent stem cell” refers to a stem cell but can only differentiate into a limited number of types. For example, the bone marrow contains multipotent stem cells that give rise to all the cells of the blood but not to other types of cells. Multipotent stem cells are found in adult animals; perhaps most organs in the body (e.g., brain, liver) contain them where they can replace dead or damaged cells. These adult stem cells may also be the cells that—when one accumulates sufficient mutations—produce a clone of cancer cells.

As used herein, the term “hepatocyte-like cell” refers to a cell that has been transfected with exogenous copies of HNF-4-α, HNF-3β, HNF-1α and/or CEBP alpha, and have been differentiated to exhibit major characteristics of a hepatocyte such as glycogen synthesis, albumin urea and bile syntheses.

As used herein, the term “exogenous copy” of a gene refers to the non-genomic copy of a gene, an added copy of gene that is introduced into the cell. The term “endogenous” use herein means the original copy of the gene found in the genome of the cell.

As used herein, the term “vector” or “plasmid” refers broadly to any plasmid, phagemid or virus encoding an exogenous nucleic acid. The term is also to be construed to include non-plasmid, non-phagemid and non-viral compounds which facilitate the transfer of nucleic acid into virions or cells, for example, poly-lysine compounds and the like. The vector can be a viral vector that is suitable as a delivery vehicle for delivery of the nucleic acid, or mutant thereof, to a cell, or the vector can be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94: 12744-12746). Examples of viral vectors include, but are not limited to, a recombinant Vaccinia virus, a recombinant adenovirus, a recombinant retrovirus, a recombinant adeno-associated virus, a recombinant avian pox virus, and the like (Cranage et al., 1986, EMBO J. 5: 3057-3063; International Patent Application No. WO94/17810, published Aug. 18, 1994; International Patent Application No. WO94/23744, published Oct. 27, 1994). Examples of non-viral vectors include, but not limited to, liposomes, polyamine derivatives of DNA, and the like.

The term “replication incompetent” as used herein means the viral vector cannot further replicate and package its genomes. For example, when the cells of a subject are infected with replication incompetent recombinant adeno-associated virus (rAAV) virions, the heterologous (also known as transgene) gene is expressed in the patient's cells, but, the rAAV is replication defective (e.g., lacks accessory genes that encode essential proteins from packaging the virus) and viral particles cannot be formed in the patient's cells.

The term “gene” means the nucleic acid sequence which is transcribed (DNA) and translated (mRNA) into a polypeptide in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

The term “nucleic acid” refers to deoxyribonucleotides or ribonucleotides and polymers thereof (“polynucleotides”) in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid molecule/polynucleotide also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J. Biol. Chem. 260: 2605-2608 (1985); Rossolini et al., Mol. Cell. Probes 8: 91-98 (1994)). Nucleotides are indicated by their bases by the following standard abbreviations: adenine (A), cytosine (C), thymine (T), and guanine (G).

The term “isolated” as used herein signifies that the cells are placed into conditions other than their natural environment. The term “isolated” does not preclude the later use of these cells thereafter in combinations or mixtures with other cells.

As used herein, the term “expanding” refers to increasing the number of like cells through cell division (mitosis). The term “proliferating” and “expanding” are used interchangeably.

As used herein, the term “transduced” refers to the transfection with exogenous copies of HNF-4-α, HNF-3β, HNF-1α and/or CEBP alpha into cells.

As used herein the term “comprising” or “comprises” is used in reference to BAL, tissue engineered construct or methods etc, and respective component(s) thereof, that are essential to the invention, yet open to the inclusion of unspecified elements, whether essential or not.

The term “consisting of” refers to BAL, tissue engineered construct or methods etc, and respective components thereof as described herein, which are exclusive of any element not recited in that description of the embodiment.

Embodiments of the present invention is based on the discovery that it is possible to induce somatic adult stem cells to differentiate into hepatocytes by the over-expression of a key nuclear transcription factor, hepatocyte nuclear factor-4-α (HNF4-α), that is essential for hepatocyte differentiation, in the somatic adult stem cells. This method serve to provide an abundant and renewable supply of hepatocytes for tissue engineering constructs, BAL, and research.

HNF-4-α was originally identified as an activity in crude rat liver nuclear extracts that bound DNA elements required for the transcription of two liver-specific genes—transthyretin (TTR) and apolipoprotein CIII (apoCIII). After protein purification and cloning, it was found that HNF4 is a member of the nuclear receptor superfamily (NR2A1) of ligand-dependent transcription factors (Sladek et al., 1990, Genes Dev. 1990 4:2353-65). In addition to a relatively high level of expression in the liver, HNF4α mRNA and protein is also found in the kidney, intestine and colon and to a lesser extent pancreas and stomach. HNF4α is expressed early in development, is required for the proper complete gastrulation of the visceral endoderm and the expression of a large array of genes whose expression in differentiated hepatocytes is essential for a functional hepatic parenchyma, including genes encoding several apolipoproteins, metabolic proteins, and serum factors. HNF-4α is central to the maintenance of hepatocyte differentiation and is a major in vivo regulator of genes involved in the control of lipid homeostasis.

The inventors introduced additional gene copies of HNF4-α in a doxycycline-inducible plasmid into adipose-derived mesenchymal stem cells (ADMSCs) using a lenti-viral vector system so as to over-express of HNF4-α in the stem cells. The transduced ADMSCs were cultured two-dimensionally and embedded between two layers of collagen hydrogel in vitro. The exogeneous HNF4-α was induced to over-express in the transduced ADMSCs by the addition of doxycycline. Following the over-expression of HNF4-α, the inventors cultured these cells in epithelial growth factor (EGF), basic fibroblast growth factor (bFGF), hepatocyte growth factor (HGF), nicotinamide, and oncostatin M (OSM) which led to the differentiation of these ADMSCs into hepatocyte-like cells exhibiting the expression of many hepatocyte specific proteins and by-products. A specific time-course regime of culturing these transfuced stem cells in culture media was developed by the inventors. More specifically, the transduced and over-expressing HNF4-α ADMSCs that were embedded in collagen hydrogel were initially cultured for 1-7 days in culture media with EGF and bFGF, followed by 1-7 days in culture media with bFGF, HGF, and nicotinamide, and finally 1-180 days in culture media with oncostatin M (OSM) to produced ADMSC derived hepatocyte cells.

These ADMSC derived hepatocyte cells express hepatocyte-specific markers: HNF4-α, CYP 3A4 (a liver-specific enzyme) and ZO-1 (zona occludens-1 tight junctions between liver cells). In addition, these ADMSC derived hepatocyte cells express mRNAs and proteins of five key metabolic pathways found in the liver: glucose-6-phosphatase for glucose metabolism; albumin for protein synthesis; arginase I for physiologic detoxification (urea production); CYP 3A4 for metabolic detoxification; and bile production.

Accordingly, gene transfer can be used to facilitate the over-expression of genes associated with hepatocyte differentiation and function in any undifferentiated stem cells. Genes associated with hepatocyte differentiation and function include but not limited to HNF-4-α, HNF-1α, HNF-3β (FOXA2), and CCAAT/enhancer binding protein (C/EBP) beta, (CEBP/α). In contrast to the endogenous expression of liver-specific genes, using gene transfer to introduce exogenous copies for over-expression purposes ensures the transduced stem cells will express the necessary genes required for hepatocyte differentiation and function. Moreover any undifferentiated stem cells (embryonic, pluripotent or multipotent) can be used for the gene transfer.

Accordingly, in one embodiment of the invention herein provides a method of producing hepatocyte-like cells from stem cells, the method comprising (a) introducing an exogenous copy of at least one gene selected from the group consisting of hepatic nuclear factor-4-α (HNF-4-α), hepatic nuclear factor-3β (HNF-3β), hepatic nuclear factor-1α (HNF-1α), and CCAAT/enhancer binding protein beta (CEBP alpha), into a stem cell; (b) ex-vivo culturing the stem cell of step (a) in a culture medium comprising epidermal growth factor (EGF) and basic fibroblast growth factor (bFGF) for 1-7 days; (c) ex-vivo culturing the stem cell of step (b) in a culture medium comprising hepatocyte growth factor (HGF), basic fibroblast growth factor (bFGF) and nicotinamide for 1-7 days; and (d) ex-vivo culturing the stem cell of step (c) in a culture medium comprising oncostatin M (OSM) for 1-180 days; wherein the method produces hepatocyte-like cells, wherein the hepatocyte-like cells have at least three of the following characteristics: glucose-6-phosphatase for glucose metabolism; albumin secretion—protein synthesis; arginase Type I—urea cycle; CYP 3A4—xenobiotic detoxification; and biliary fluorescence assay (morphologic assay)—biliary secretion.

In one embodiment, the transduced stem cells are embedded in an extracellular matrix, such as collagen. The transduced stem cells are grown two-dimensionally in a monolayer in the extracellular matrix.

In some embodiments, the methods described herein comprise providing a supply of embryonic, pluripotent or multipotent stem cells cultured in vitro. In some embodiments, the stem cell is an adult, fetal or embryonic stem cell. In other embodiments, differentiated cells that have been reprogrammed into stem cells can also be used for the method of producing differentiated hepatocytes from stem cells. In other embodiments, the stem cell is isolated from umbilical, placenta, amniotic fluid, chorion villi, blastocysts, bone marrow, adipose tissue, brain, peripheral blood, blood vessels, skeletal muscle, skin and liver. In other embodiments, the cells are pluripotent stem cells or dermal fibroblasts.

In one embodiment, the exogenous copy of the gene of is introduced into the stem cell in an inducible plasmid such as a doxycycline-inducible plasmid. For example, pcDNA™4/TO and pcDNA™5/TO plasmid from Invitrogen Inc. These inducible expression plasmids are suitable for the expression of a gene of interest under the control of the strong human cytomegalovirus immediate-early (CMV) promoter and two tetracycline operator 2 (TetO2) sites. A regulatory plasmid, pcDNA6/TR©, which encodes the Tet repressor (TetR) under the control of the human CMV promoter is also introduced into the stem cells. The TetR produced forms a homodimer that binds with extremely high affinity to each TetO2 sequence in the promoter of the inducible expression vector, blocking transcription. In the presence of doxycycline or tetracycline, the repression by TetR is removed, and the gene of interest is expressed.

In one embodiment, the exogenous copy of the gene of HNF-4-α, HNF-3β, HNF-1α, and/or CEBP/α is introduced into the stem cell in a non-inducible plasmid such as a plasmid where gene expression is constitutive, under the control of the human CMV promoter.

In some embodiments, the vectors carrying the exogenous copy of HNF-4-α, HNF-3β, HNF-1α and/or CEBP/α can be viral or non-viral. In some embodiments, the vectors carrying the exogenous copy of HNF-4-α, HNF-3β, HNF-1α, and/or CEBP/α can be introduced into mammalian host cells by standard transfection methods.

In some embodiments, the method described herein further comprises embedding and encapsulating the transduced stem cells in a collagen to form a collagen sandwich culture. Collagen hydrogel preparations are pH measured and corrected to be within a range of 7.4 to 7.9, and plated in vessels in sufficient liquid volumes to achieve a hydrogel base layer thickness of ≃150 μm. The liquid gels are then allowed to set for 30 minutes at 37° C. in 5% CO₂ atmosphere. Transduced stem cells are then plated at a density of ˜35,000 cells per cm². The transduced stem cells are allowed to attach overnight and the culture media is removed. The cells are gently washed to remove cellular debris and a second collagen hydrogel is applied to the top of the cells. The second hydrogel is allowed to set at 30 minutes at 37° C. in 5% CO₂ atmosphere. In other embodiments, other hydrogel materials such as gelatin, fibrinogen, chitosan, hylauronic acid, alginate, poly-ethyleneglycol, lactic acid, and N-isopropyl arcrylamide can be used.

In another embodiment, the invention herein provides a method of producing hepatocyte-like cells from stem cells, the method comprising (a) increasing the expression of at least one gene selected from the group consisting of hepatic nuclear factor-4-α (HNF-4-α), hepatic nuclear factor-3β (HNF-3β), hepatic nuclear factor-1α (HNF-1α), and CCAAT/enhancer binding protein beta (CEBP alpha), into a stem cell; (b) ex-vivo culturing the stem cell of step (a) in a culture medium comprising epidermal growth factor (EGF) and basic fibroblast growth factor (bFGF) for 1-7 days; (c) ex-vivo culturing the stem cell of step (b) in a culture medium comprising hepatocyte growth factor (HGF), basic fibroblast growth factor (bFGF) and nicotinamide for 1-7 days; and (d) ex-vivo culturing the stem cell of step (c) in a culture medium comprising oncostatin M (OSM) for 1-180 days; wherein the method produces hepatocyte-like cells, wherein the hepatocyte-like cells have at least three of the following characteristics: glucose-6-phosphatase for glucose metabolism; albumin secretion—protein synthesis; arginase Type I—urea cycle; CYP 3A4—xenobiotic detoxification; and biliary fluorescence assay (morphologic assay)—biliary secretion.

One of ordinary skill in the art should be able to increase gene expression of the endogenous gene by any method of known it the art. For example, in U.S. Pat. No. 5,939,541 which is hereby incorporated by reference in its entirety.

Unless otherwise explained, all technical and scientific terms used herein have the same meaning as commonly understood by one of ordinary skill in the art to which this disclosure belongs. Definitions of common terms in medicine, cell and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); The ELISA guidebook (Methods in molecular biology 149) by Crowther J. R. (2000); Fundamentals of RIA and Other Ligand Assays by Jeffrey Travis, 1979, Scientific Newsletters; Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

It should be understood that this invention is not limited to the particular methodology, protocols, and reagents, etc., described herein and as such may vary. The terminology used herein are for the purpose of describing particular embodiments only, and is not intended to limit the scope of the present invention, which is defined solely by the claims.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Stem Cells Useful for the Invention

In some embodiments, the stem cells useful for the method described herein include but not limited to embryonic, fetal and adult stem cells. In other embodiments, the stem cells useful for the method described herein are pluripotent or multi-potent stem cells.

Stem cells can undergo self-renewing cell division to give rise to phenotypically and genotypically identical daughters for an indefinite time and ultimately can differentiate into at least one final cell type. Stem cells are defined as cells that have extensive, and perhaps indefinite, proliferation potential that differentiate into several cell lineages, and that can repopulate tissues upon transplantation. The quintessential stem cell is the embryonic stem (ES) cell, as it has unlimited self-renewal and multipotent differentiation potential. These cells are derived from the inner cell mass of the blastocyst, or can be derived from the primordial germ cells from a post-implantation embryo (embryonic germ cells or EG cells). ES and EG cells have been derived from mouse, non-human primates and humans. When introduced into mouse blastocysts or blastocysts of other animals, ES cells can contribute to all tissues of the mouse (animal). When transplanted in post-natal animals, ES and EG cells generate teratomas, which again demonstrates their multipotency. ES (and EG) cells can be identified by positive staining with the antibodies SSEA1 and SSEA4.

At the molecular level, ES and EG cells are characterized by the expression of a number of transcription factors highly specific for these undifferentiated cells. These include Oct-4, Rex-1, Sox-2 the LIF-R and Rox-1. Oct-4 is required for maintaining the undifferentiated phenotype of ES cells, and plays a major role in determining early steps in embryogenesis and differentiation. oct-4, in combination with Rox-1, causes transcriptional activation of the Zn-finger protein Rex-1, and is also required for maintaining ES in an undifferentiated state. Likewise, sox-2, is needed together with oct-4 to retain the undifferentiated state of ES/EC. Human primordial germ cells require presence of LIF. In addition, ES cells have telomerase, which provides these cells with an unlimited self-renewal potential in vitro.

Fetal stem cells are cells with self-renewal capability and pluripotent differentiation potential. They can be isolated and expanded from fetal cytotrophoblast cells (European Patent EPO412700) and chorionic villi, amniotic fluid and the placenta (WO/2003/042405). These are hereby incorporated by reference in their entirety. Cell surface markers of fetal stem cells include CD117/c-kit+, SSEA3+, SSEA4+ and SSEA1−.

Somatic stem cells have been identified in most organ tissues. The best characterized is the hematopoietic stem cell. This is a mesoderm-derived cell that has been purified based on cell surface markers and functional characteristics. The hematopoietic stem cell, isolated from bone marrow, blood, cord blood, fetal liver and yolk sac, is the progenitor cell that reinitiates hematopoiesis for the life of a recipient and generates multiple hematopoietic lineages (see U.S. Pat. Nos. 5,635,387; 5,460,964; 5,677,136; 5,750,397; 759,793; 5,681,599; 5,716,827; Hill, B., et al., Exp. Hematol. (1996) 24 (8): 936-943). These are hereby incorporated by reference in their entirety. When transplanted into lethally irradiated animals or humans, hematopoietic stem cells can repopulate the erythroid, neutrophil-macrophage, megakaryocyte and lymphoid hematopoieticcell pool. In vitro, hematopoieticstem cells can be induced to undergo at least some self-renewing cell divisions and can be induced to differentiate to the same lineages as is seen in vivo. Therefore, this cell fulfills the criteria of a stem cell.

The next best characterized is the mesenchymal stem cells (MSC), originally derived from the embryonic mesoderm and isolated from adult bone marrow, can differentiate to form muscle, bone, cartilage, fat, marrow stroma, and tendon. During embryogenesis, the mesoderm develops into limb-bud mesoderm, tissue that generates bone, cartilage, fat, skeletal muscle and possibly endothelium. Mesoderm also differentiates to visceral mesoderm, which can give rise to cardiac muscle, smooth muscle, or blood islands consisting of endothelium and hematopoietic progenitor cells. Primitive mesodermal or mesenchymal stem cells, therefore, could provide a source for a number of cell and tissue types. A number of mesenchymal stem cells have been isolated (see, for example, U.S. Pat. Nos. 5,486,359; 5,827,735; 5,811,094; 5,736,396; U.S. Pat. Nos. 5,837,539; 5,837,670; 5,827,740; Jaiswal, N., et al., J. Cell Biochem. (1997) 64(2): 295-312; Cassiede P., et al., J. Bone Miner. Res. (1996) 11(9): 1264-1273; Johnstone, B., et al., (1998) 238(1): 265-272; Yoo, et al., J. Bone Joint Sure. Am. (1998) 80(12): 1745-1757; Gronthos, S., Blood (1994) 84(12): 41644173; Makino, S., et al., J. Clin. Invest. (1999) 103(5): 697-705). These are hereby incorporated by reference in their entirety. Of the many mesenchymal stem cells that have been described, all have demonstrated limited differentiation to form only those differentiated cells generally considered to be of mesenchymal origin. To date, the most multipotent mesenchymal stem cell expresses the SH2+ SH4+ CD29+ CD44+ CD71+ CD90+ CD106+ CD120a+ CD124+ CD14− CD34− CD45− phenotype.

In one embodiment, adipose-derived mesenchymal stem cells (ADMSCs) are used for the method described herein. ADMSCs are normally proliferative and are readily available through minimally invasive procedures. They can survive freezing and thawing and require no special handling in vitro (such as co-culture, defined media, etc.).

Other stem cells have been identified, including gastrointestinal stem cells, epidermal stem cells, neural and hepatic stem cells, also termed oval cells (Potten C, Philos Trans R Soc Lond B Biol Sci 353:821-30, 1998; Watt F, Philos. Trans R Soc Lond B Biol Sci 353:831, 1997; Alison M et al, Hepatol 29:678-83, 1998).

In some embodiments, the stem cells useful for the method described herein include but not limited to embryonic stem cell, mesenchymal stem cells, bone-marrow derived stem cells hematopoietic stem cells chrondrocytes progenitor cells, epidermal stem cells, gastrointestinal stem cells, neural stem cells, hepatic stem cells adipose-derived mesenchymal stem cells, pancreatic progenitor cells, hair follicular stem cells, endothelial progenitor cells and smooth muscle progenitor cells.

In some embodiments, differentiated cells that have been reprogrammed into stem cells are used for the method described herein. Such cells are referred to as induced pluripotent stem cells (iPS). For example, human skin cells have been reprogrammed into embryonic stem cells by the transduction of Oct3/4, Sox2, c-Myc and Klf4 (Junying Yu, et. al., 2007, Science 318: 1917-1920; Takahashi K. et. al., 2007, Cell 131: 1-12). Neural tissues were differentiated from these converted skin cells. U.S. Pat. No. 7,410,773 teaches a method of “dedifferentiating” mature cells in order to increase the ability of the cells to act like stem cells. These references are hereby incorporated by reference in their entirety. A person's own cells can be manipulated to provide a supply of hepatocytes that can be used in the manufacture of a hepatocyte-based bioartificial liver and/or tissue engineered liver or even for hepatocyte transplantation into that same person. These hepatocytes are genetically similar to the host except for the exogenously added genes and the recipient's immune system should not recognize them as foreign, transplant tissues. This will greatly reduce the incidence of tissue rejection.

In some embodiments, the stem cells used for the method described herein is isolated from umbilical cord, placenta, amniotic fluid, chorion villi, blastocysts, bone marrow, adipose tissue, brain, peripheral blood, the gastrointestinal tract, cord blood, blood vessels, skeletal muscle, skin, liver and menstrual blood. Stem cells prepared in the menstrual blood are called endometrial regenerative cells (Medistem Inc.).

One ordinary skilled artisan in the art can locate, isolate and expand such stem cells. The detailed procedures for the isolation of human stem cells from various sources are described in Current Protocols in Stem Cell Biology 2007 (Mick Bhatia, et. al., ed., John Wiley and Sons, Inc.) and it is hereby incorporated by reference in its entirety. Alternatively, commercial kits and isolation systems can be used. For example, the BD FACSAria cell sorting system, BD IMag magnetic cell separation system, and BD IMag mouse hematopoietic progenitor cell enrichment set from BD Biosciences. Methods of isolating and culturing stem cells from various sources are also described in U.S. Pat. Nos. 5,486,359, 6,991,897, 7,015,037, 7,422,736, 7,410,798, 7,410,773, 7,399,632 and these are hereby incorporated by reference in their entirety.

### Nuclear Transcription Factors Essential to the Hepatocyte Genotype and Differentiation.

The numerous functions of hepatocytes are controlled primarily at the transcriptional level by the concerted actions of a limited number of hepatocyte-enriched transcription factors, mainly hepatocyte nuclear factor 1-α (HNF-1α), -1β, -3α, -3β, -3γ, -4-α, and -6 and members of the c/ebp family). Of these, only HNF-4-α (also known as nuclear receptor 2A1 or (NR2A1)) and HNF-1α appear to be correlated with the differentiated phenotype of cultured hepatoma cells. HNF-1α-null mice are viable, indicating that this factor is not an absolute requirement for the formation of an active hepatic parenchyma. In contrast, HNF-4α-null mice die during embryogenesis. HNF4α is expressed early in development, visible by in situ hybridization in the mouse visceral endoderm at embryonic day 4.5, long before liver development. Whereas HNF4α appears to be essential in the visceral endoderm it may not be necessary for the earliest steps in the development of the fetal liver (Li et al., 2000, Genes Dev. 2000, 14:464-74). HNF-4α is both essential for hepatocyte differentiation during mammalian liver development and also crucial for metabolic regulation and proper liver function (Hayhurst et al., 2001, Mol Cell Biol. 2001 February; 21(4):1393-403).

HNF-4α is also known as TCF; HNF4; MODY; MODY1; NR2A1; TCF14; HNF4α7; HNF4α8; HNF4α9; NR2A21; and FLJ39654. Six transcriptional variants or isoforms are produced from the genomic gene, isoforms a, b c, d, d, e, and f (Genbank Accession Nos: NM_{—}000457.3 (SEQ. ID. No. 1), NM_{—}001030003.1 (SEQ. ID. No. 2), NM_{—}001030004.1 (SEQ. ID. No. 3), NM_{—}175914.3 (SEQ. ID. No. 4), NM_{—}178849.1 (SEQ. ID. No. 5), and NM_{—}178850.1 (SEQ. ID. No. 6)). All isoforms contain a zinc finger, C4 type DNA binding domain and ligand-binding domain. The encoded protein is a nuclear transcription factor which binds DNA as a homodimer and controls the expression of several genes, including HNF-1α, a transcription factor which in turns regulates the expression of several hepatic genes. Over 55 distinct target genes have been identified for HNF4α. Since many of those genes contain more than one HNF4α binding site, the total number of distinct, non species redundant HNF4α binding sites is now 74. These genes can be grouped into several different categories, according to function, such as nutrient transport and metabolism, blood maintenance, immune function, liver differentiation and growth factors. The best characterized HNF-4-α target genes are those involved in lipid transport (e.g., apolipoprotein genes) and glucose metabolism (e.g., L-PK and PEPCK). Nearly all of the target genes identified thus far are expressed primarily in the liver; several are expressed in other organs as well, such as the pancreas.

HNF-1α is also known as HNF1, LFB1, TCF1, and MODY3. HNF-1α is a transcription factor that is highly expressed in the liver and is involved in the regulation of the expression of several liver specific genes such as the human class I alcohol dehydrogenase. HNF-1α (GenbankAccession No: NM_{—}000545.4) (SEQ. ID. No. 7) belongs to the homeobox gene family for it contains a homeobox DNA binding domain. A homeobox is a DNA sequence that binds DNA. The translated homeobox is a highly conserved stretch of 60 amino acid residues. The expression of HNF-1α controlled by HNF4α. This gene has been linked to sporadic cases of maturity-onset diabetes of the young.

HNF-3β is also known as forkhead box A2 (FOXA2), HNF3B, TCF3B and MGC19807. HNF-3β is a member of the forkhead class of DNA-binding proteins. The forkhead box is a sequence of 80 to 100 amino acids that form a motif that binds to DNA. This forkhead motif is also known as the winged helix due to the butterfly-like appearance of the loops in the protein structure of the domain. These hepatocyte nuclear factors are transcriptional activators for liver-specific genes such as albumin and transthyretin, and they also interact with chromatin. Similar family members in mice have roles in the regulation of metabolism and in the differentiation of the pancreas and liver. This gene has been linked to sporadic cases of maturity-onset diabetes of the young. Transcript variants encoding different isoforms, isoform 1 and 2, have been identified for this gene (Genbank Accession Nos: NM_{—}021784.4 (SEQ. ID. No. 8) and NM_{—}153675.2 (SEQ. ID. No. 9).

CCAAT/enhancer binding protein (C/EBP) alpha is a CCAAT/enhancer-binding protein. C/EBPs are a family of transcription factors that are critical for cellular differentiation, terminal functions and inflammatory response. Six members of the family have been characterized (C/EBP alpha, C/EBP beta, C/EBP delta, C/EBP epsilon, C/EBP gamma and C/EBP zeta) and are distributed in a variety of tissues.

Transcription factors that function in hepatocyte differentiation and maintenance include any one or more of HNF1A, HNF1B, FOXA2, HNF4A; HNF6, LRH1 and CEBP/alpha. Agents useful in hepatocyte generation include dexamethasone, sodium butyrate, insulin, basic fibroblast growth factor (bFGF), hepatocyte growth factor (HGF), and activin A.

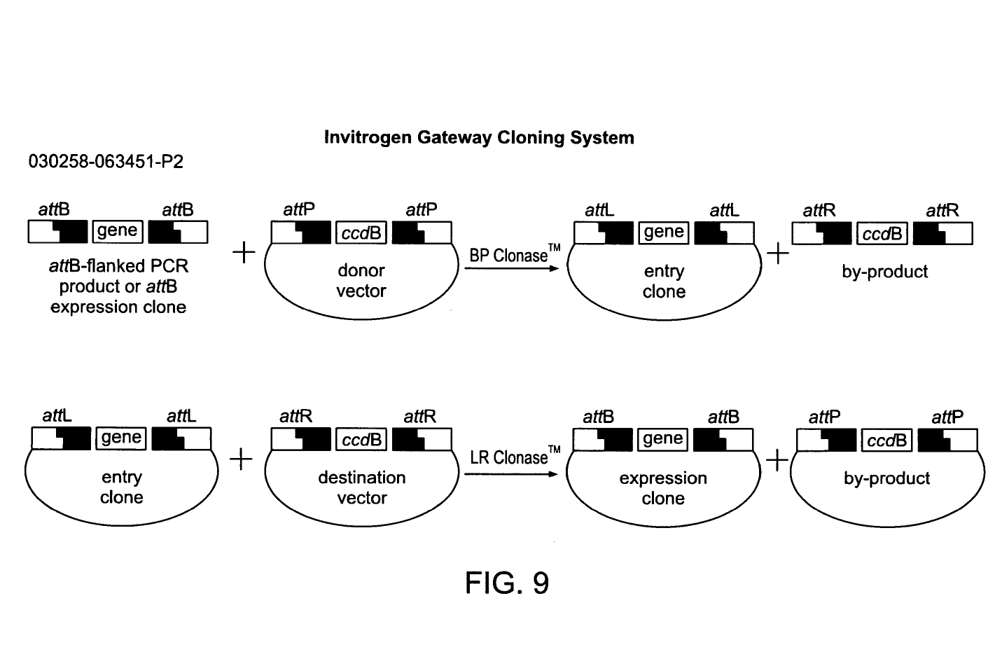

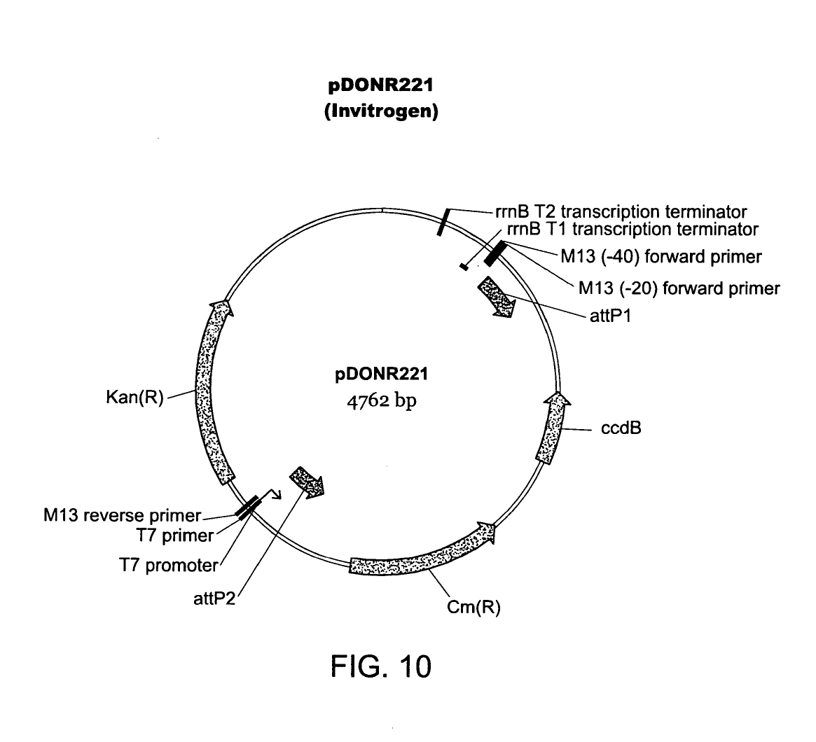

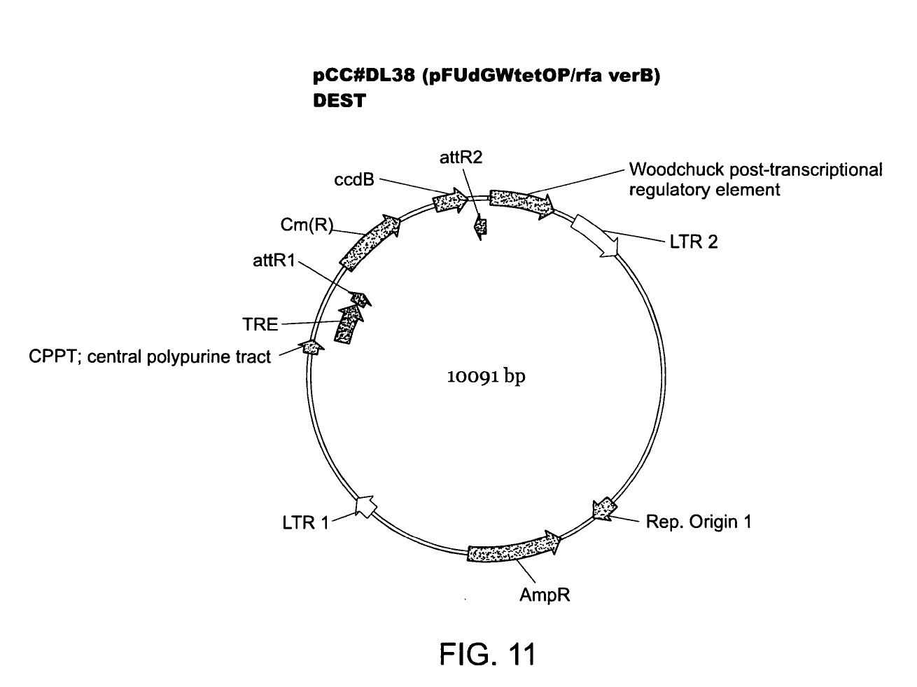

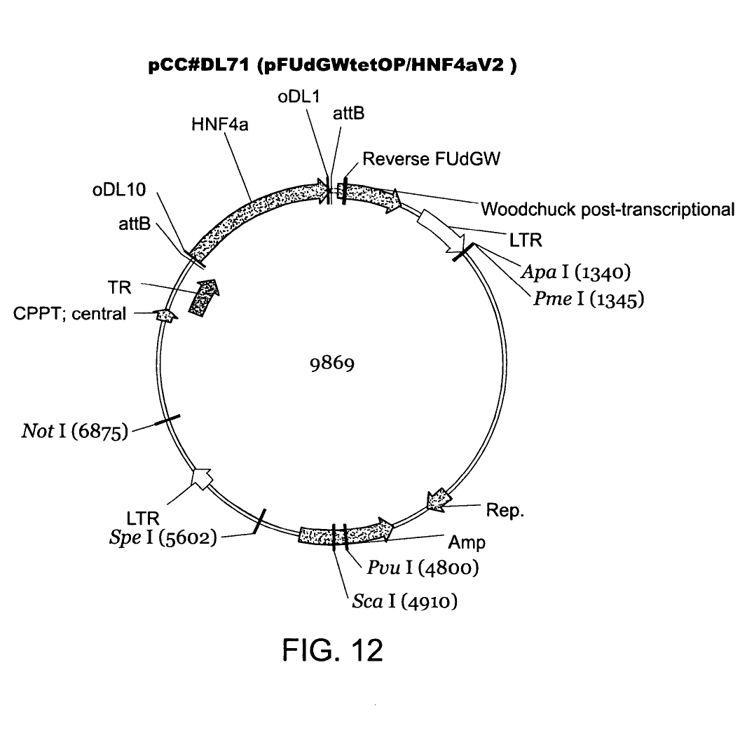

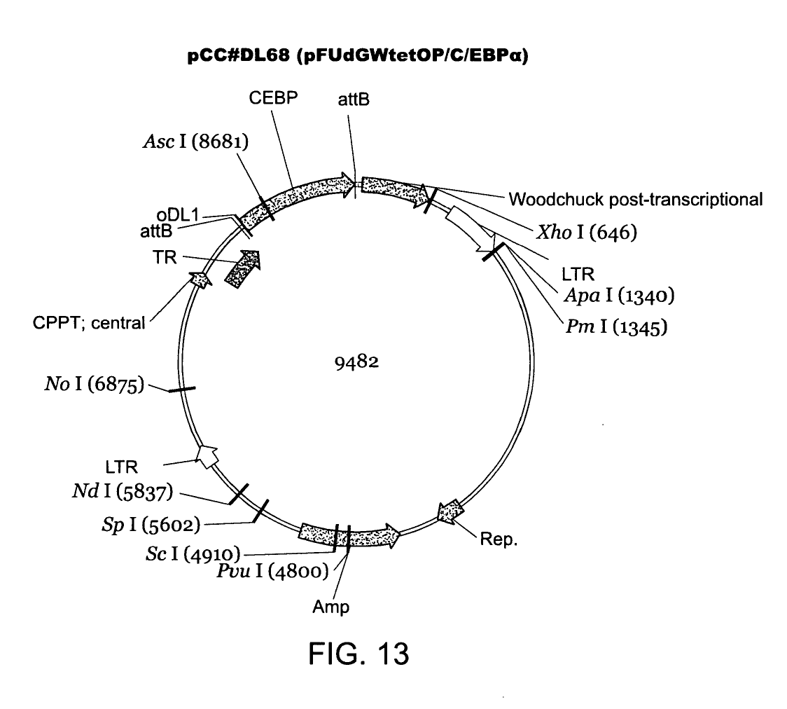

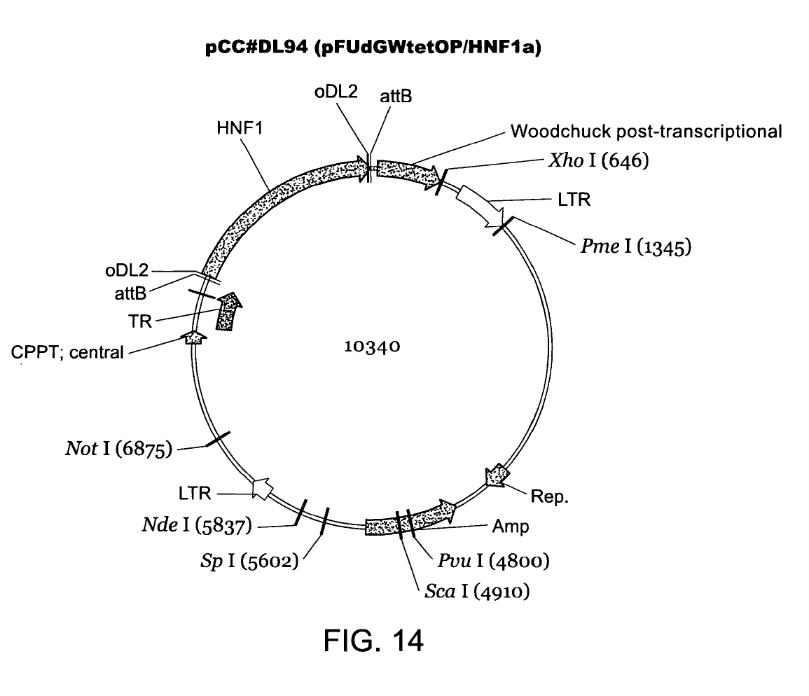

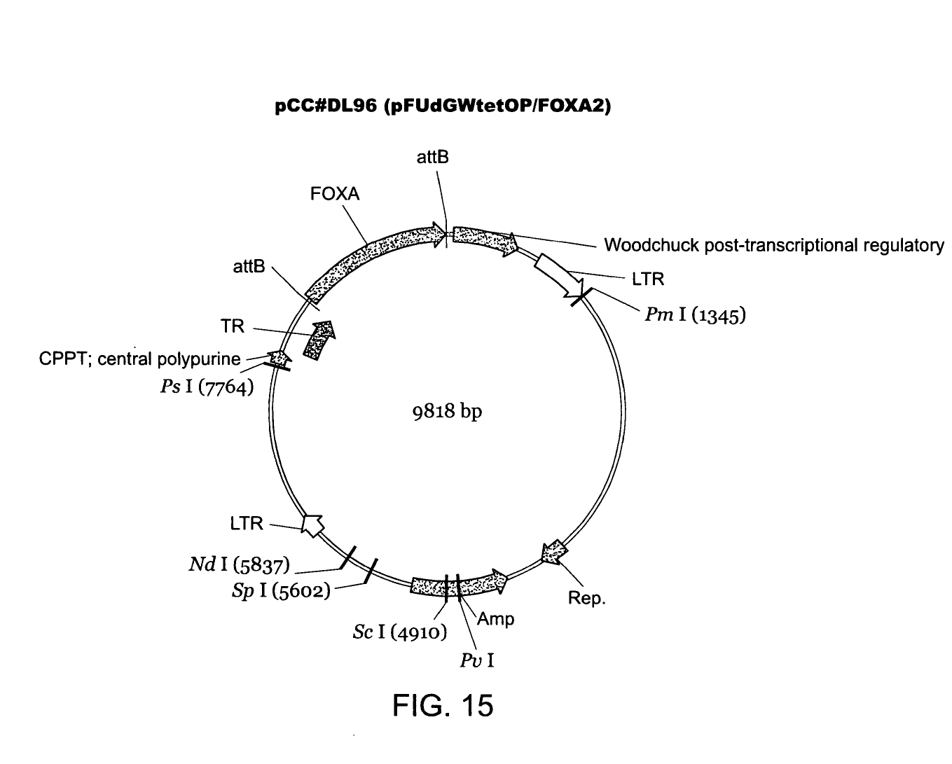

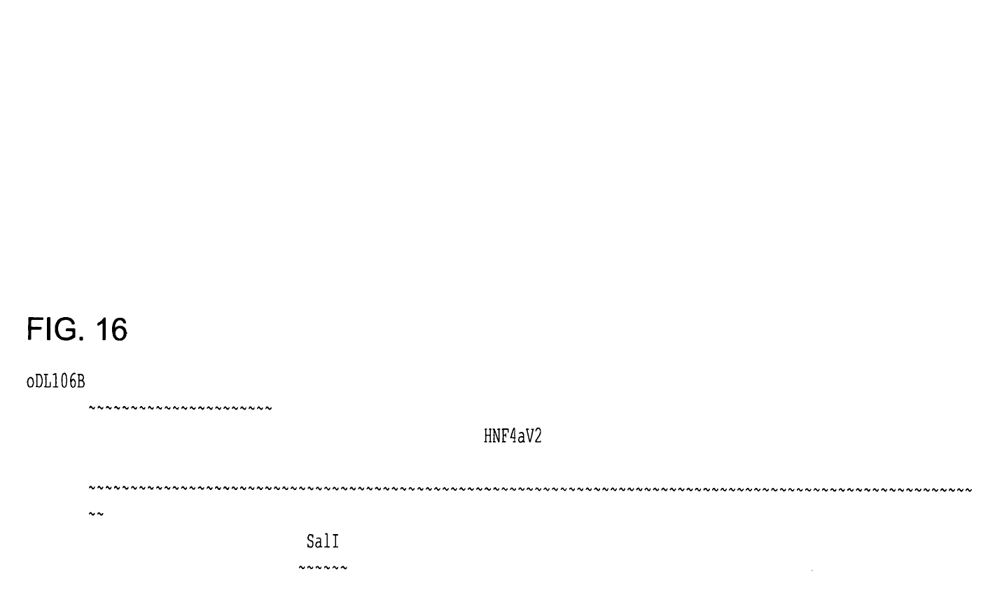

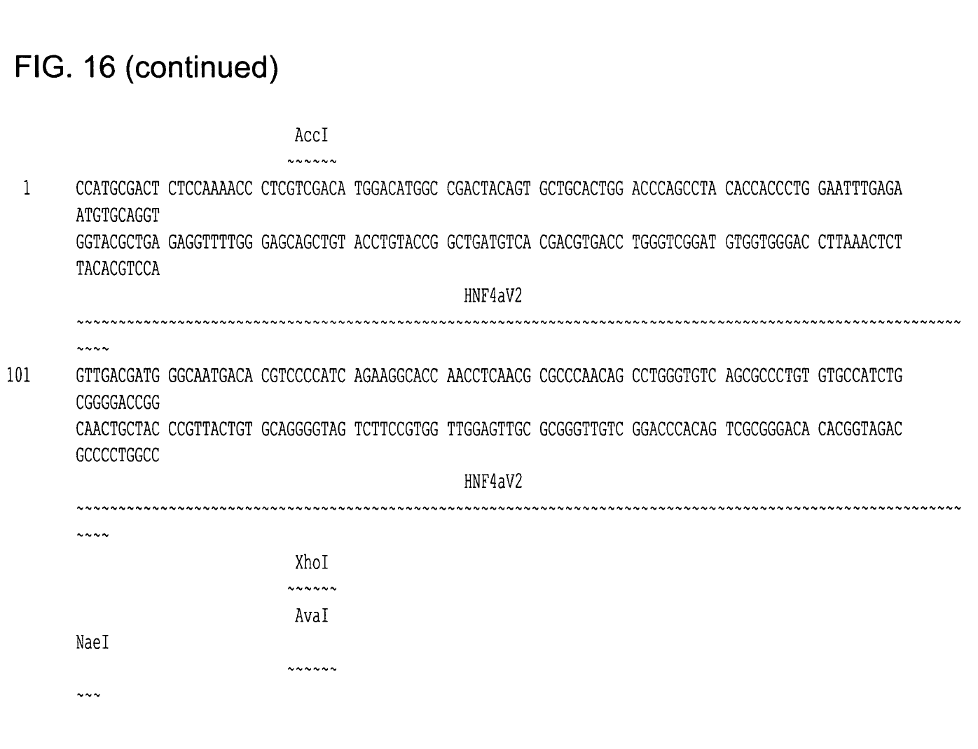

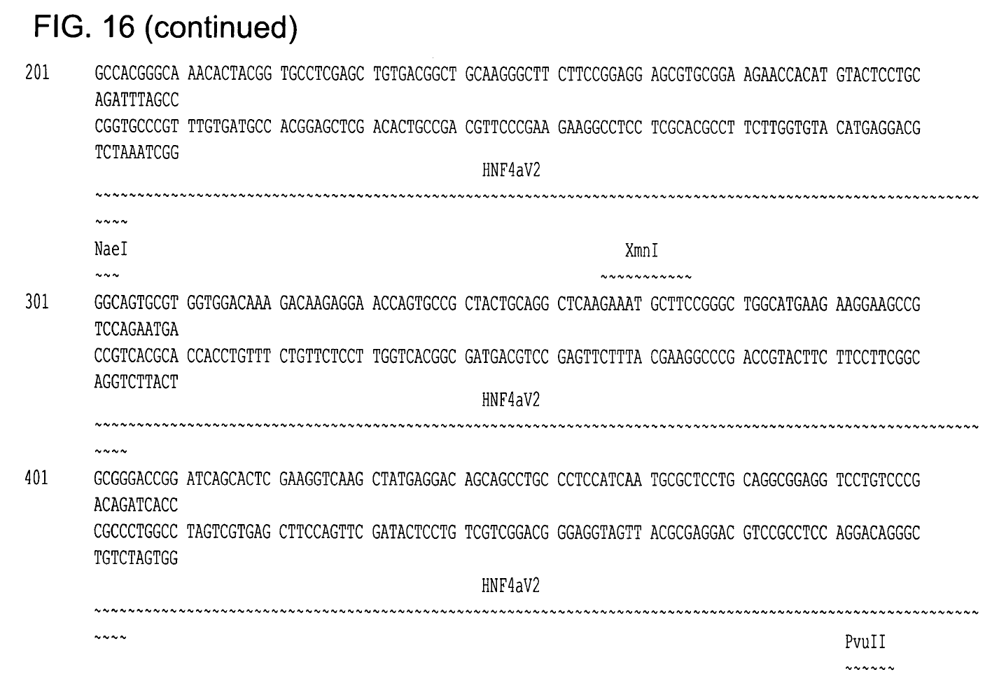

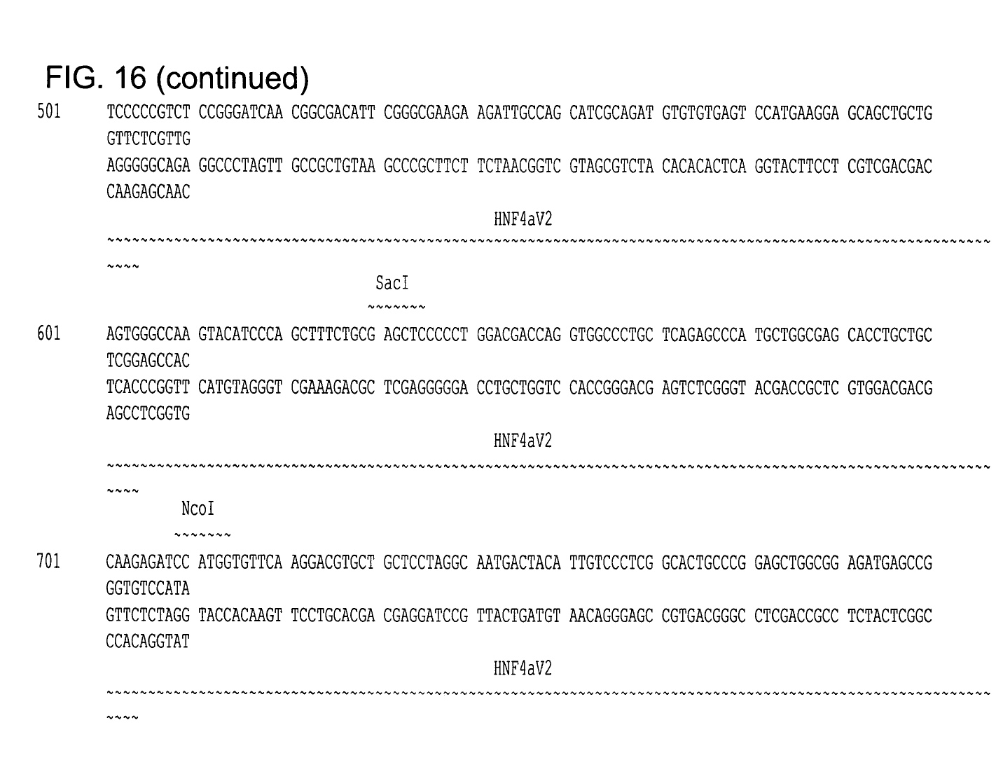

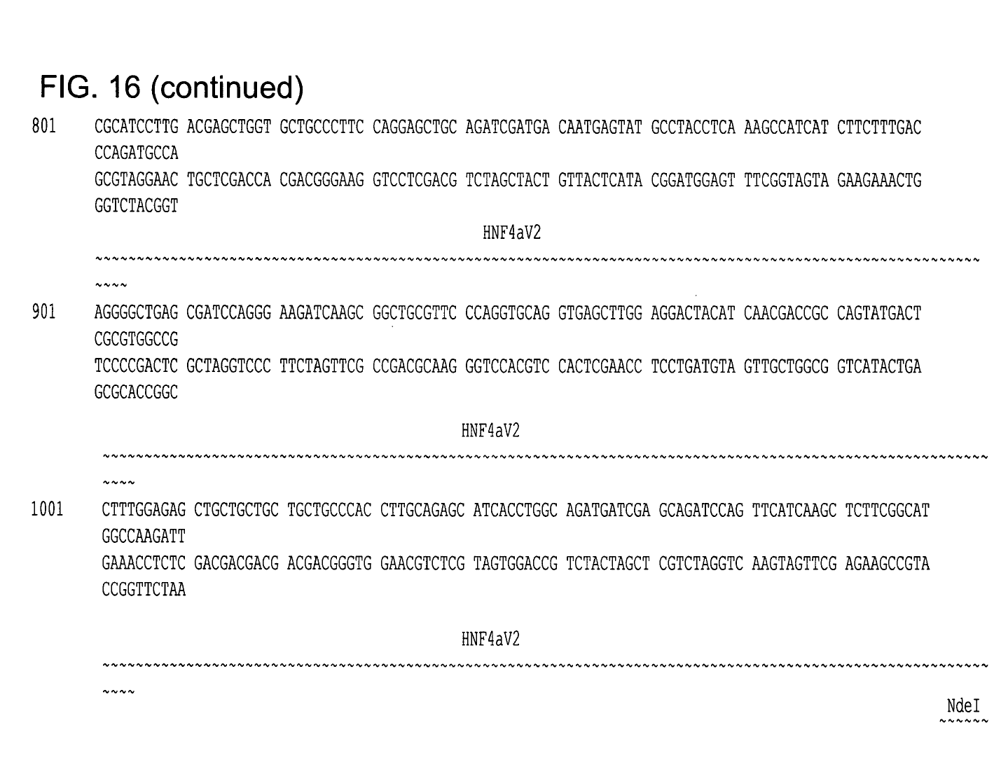

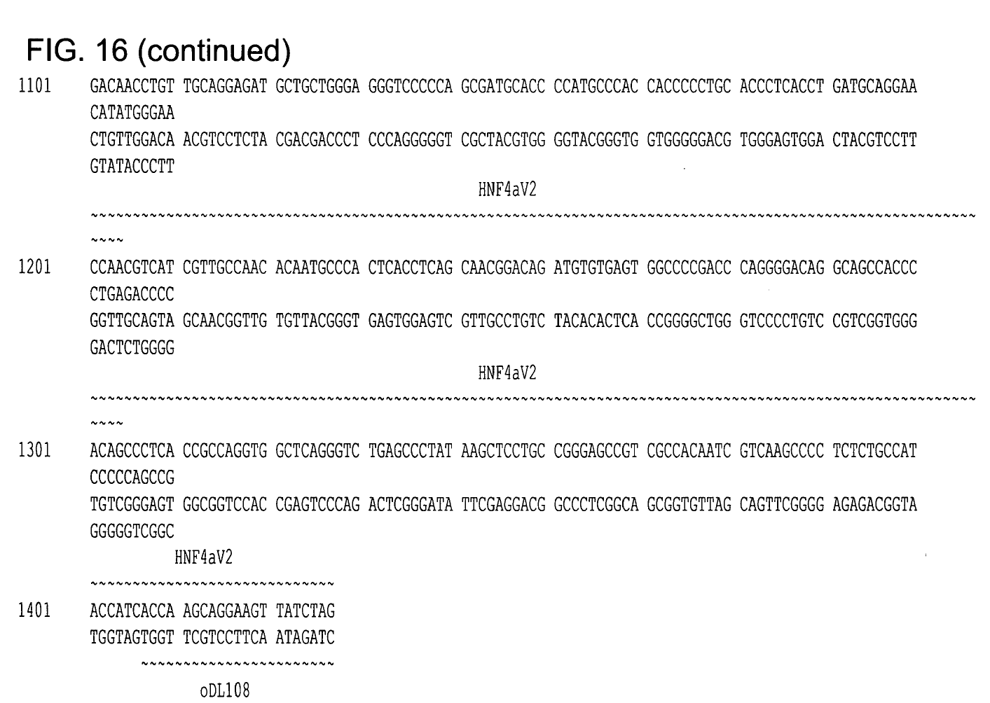

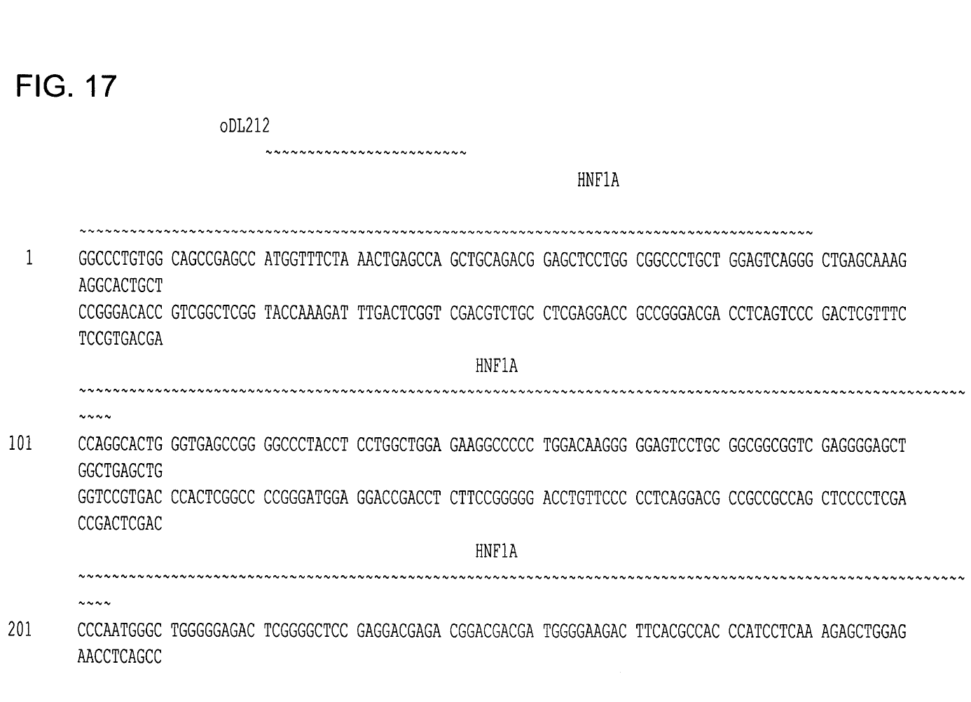

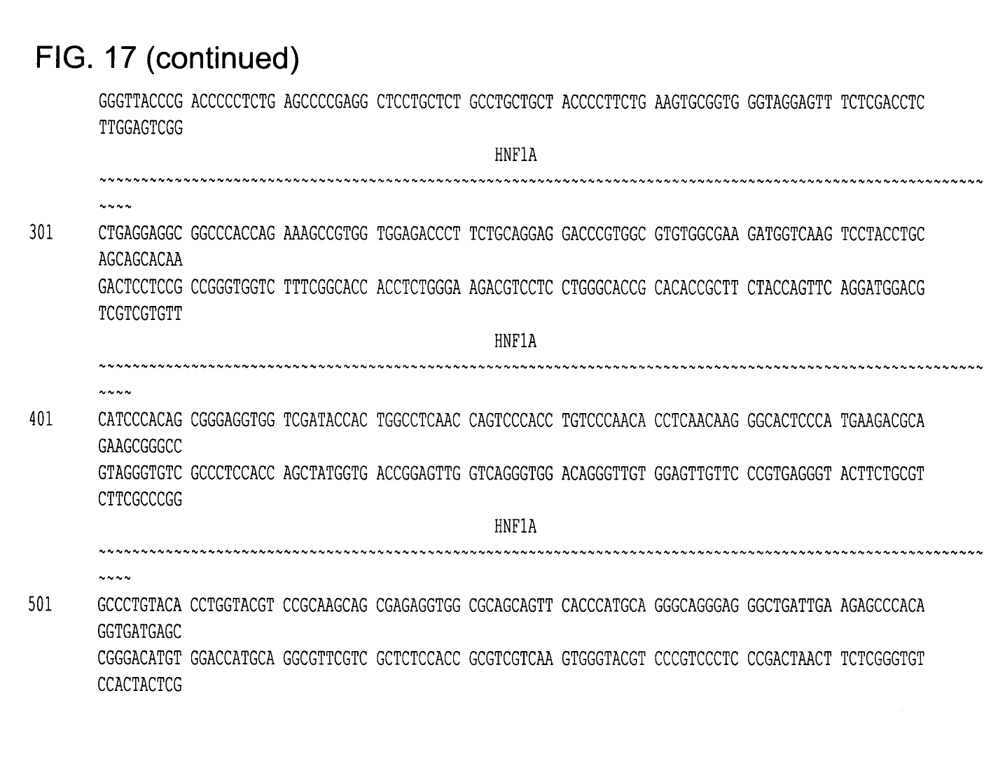

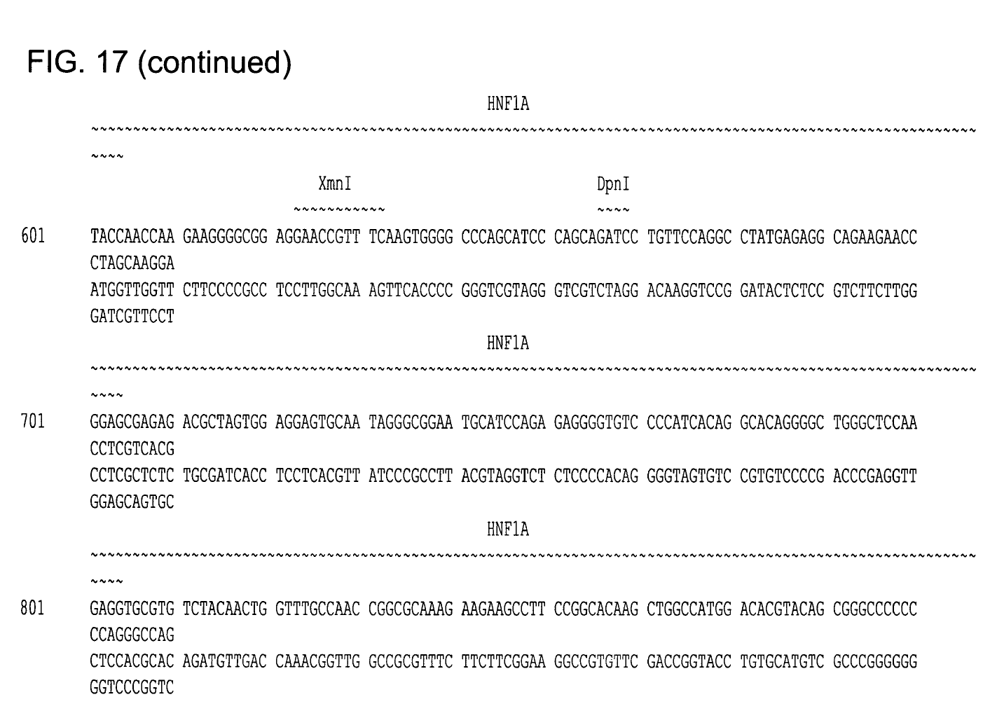

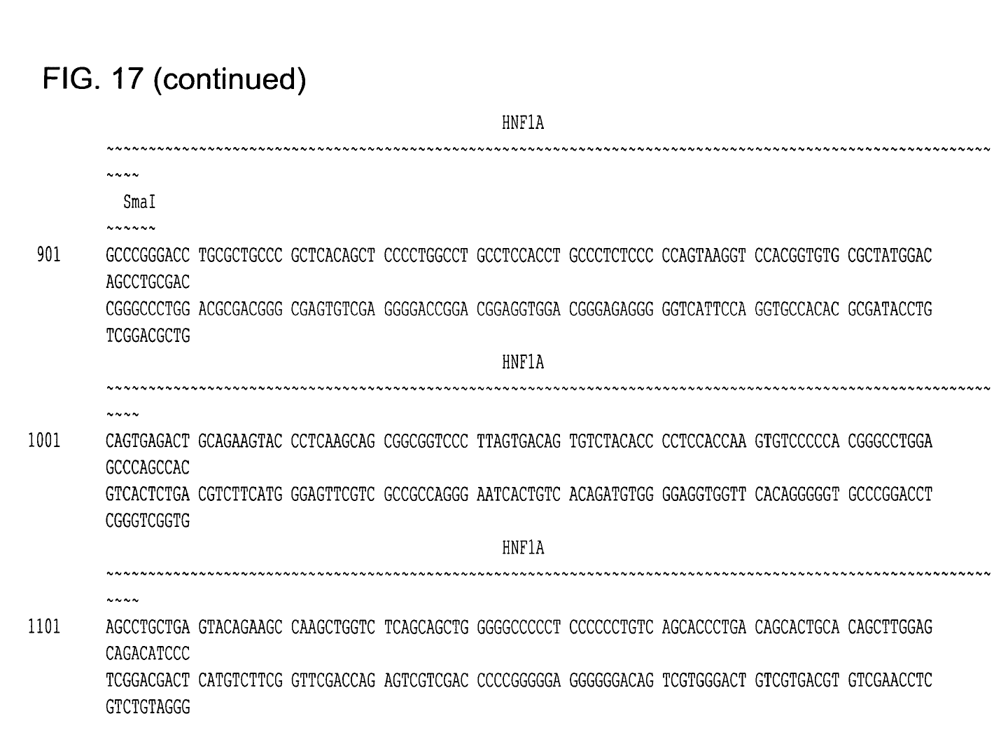

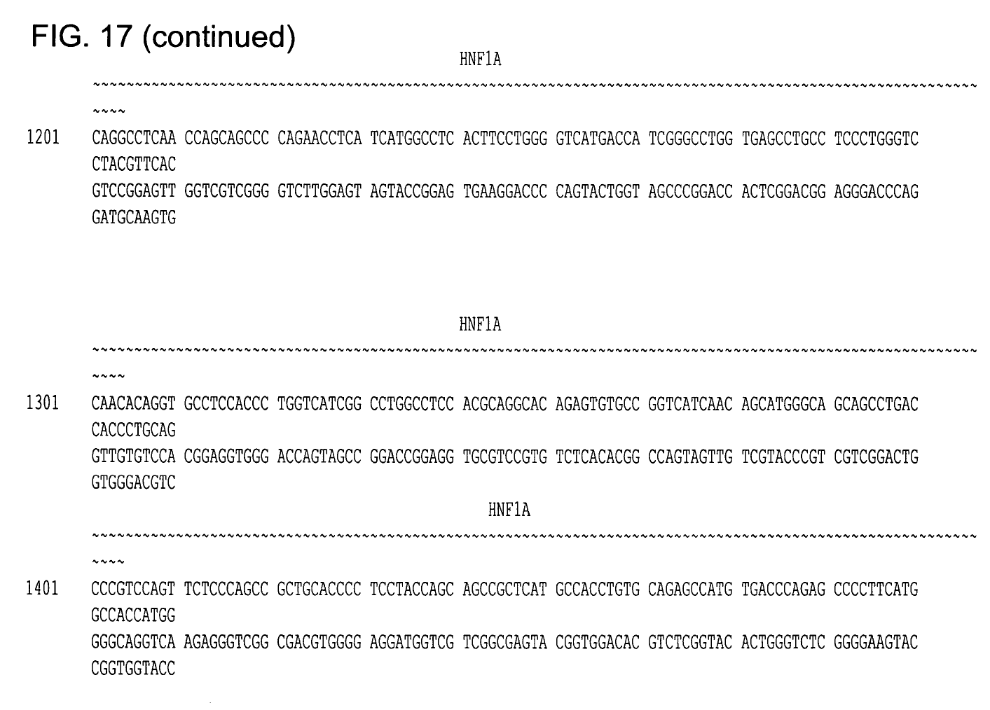

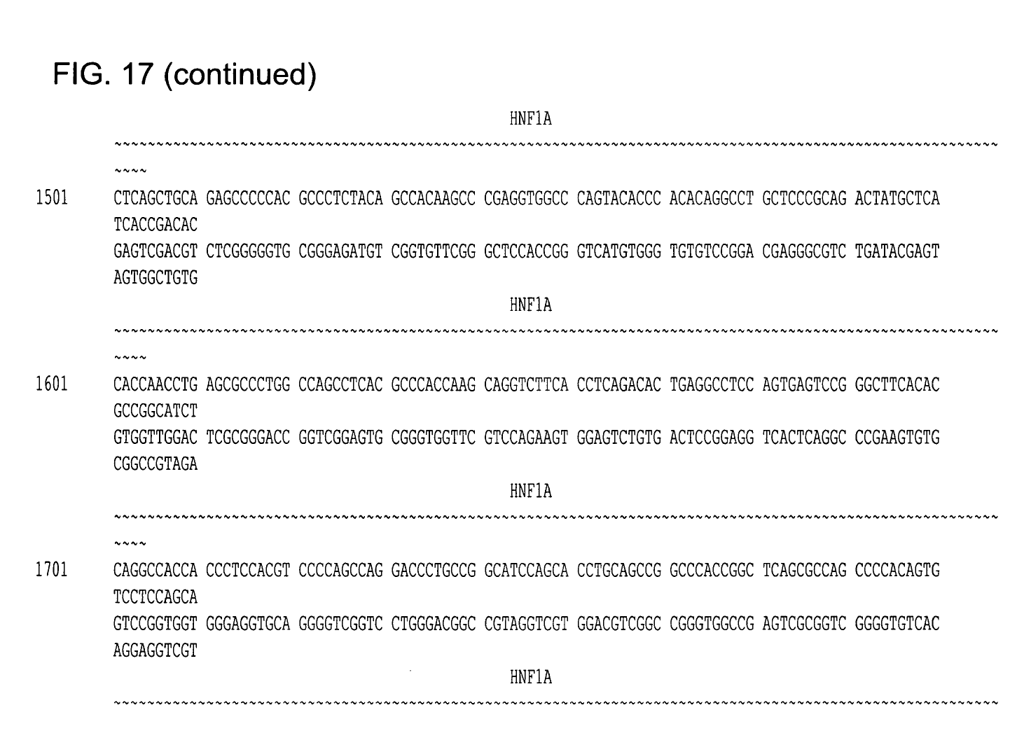

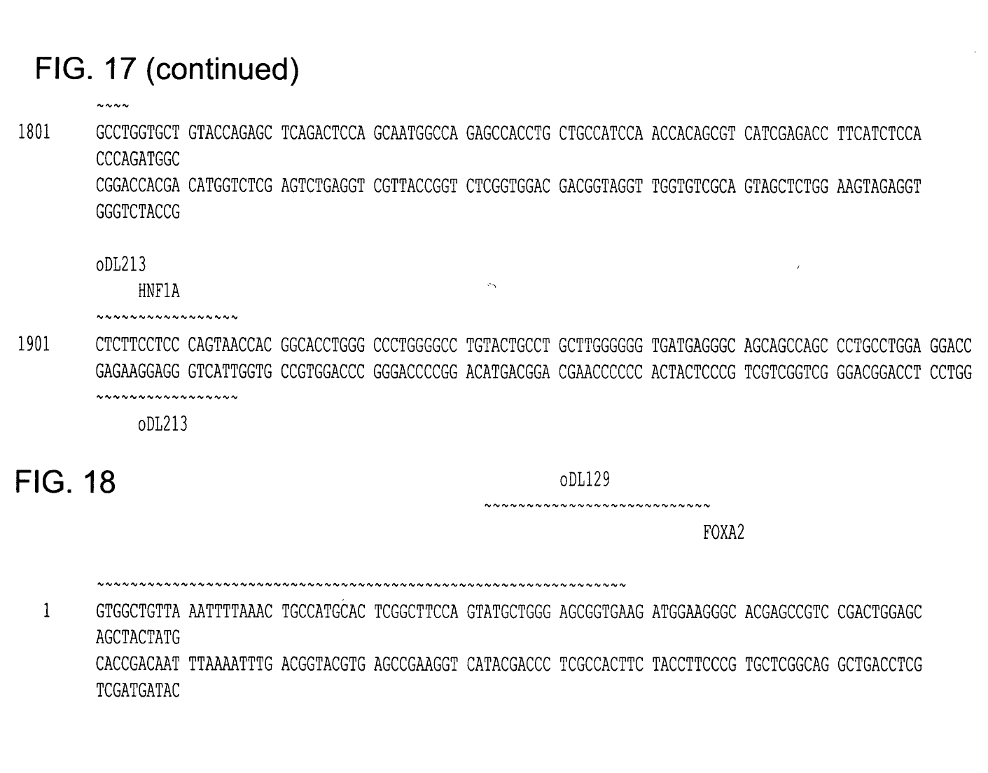

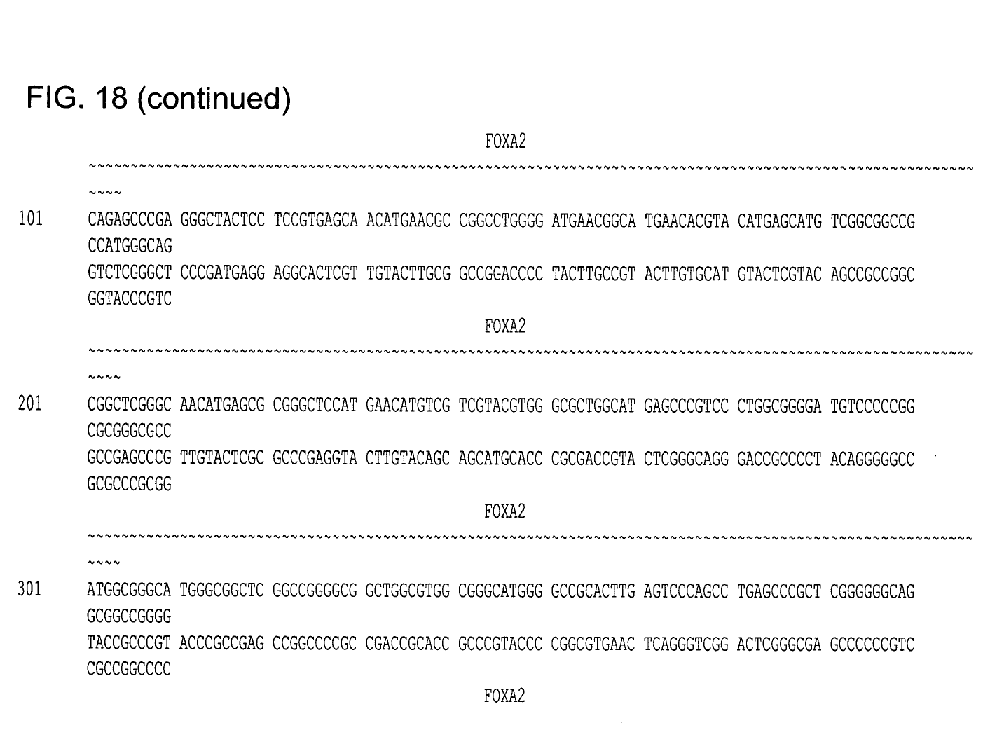

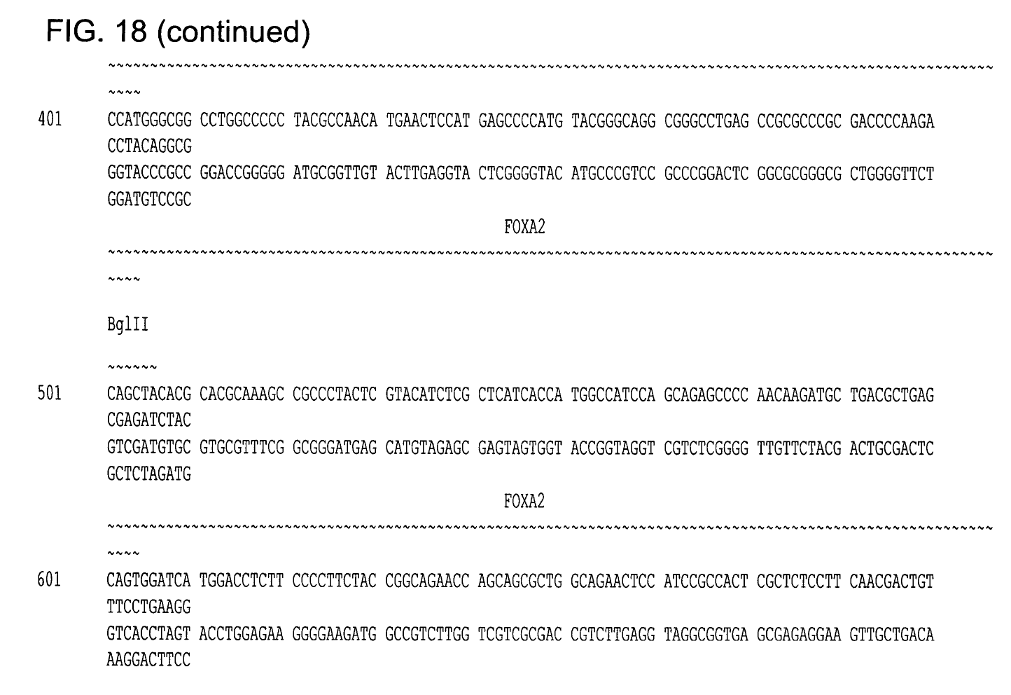

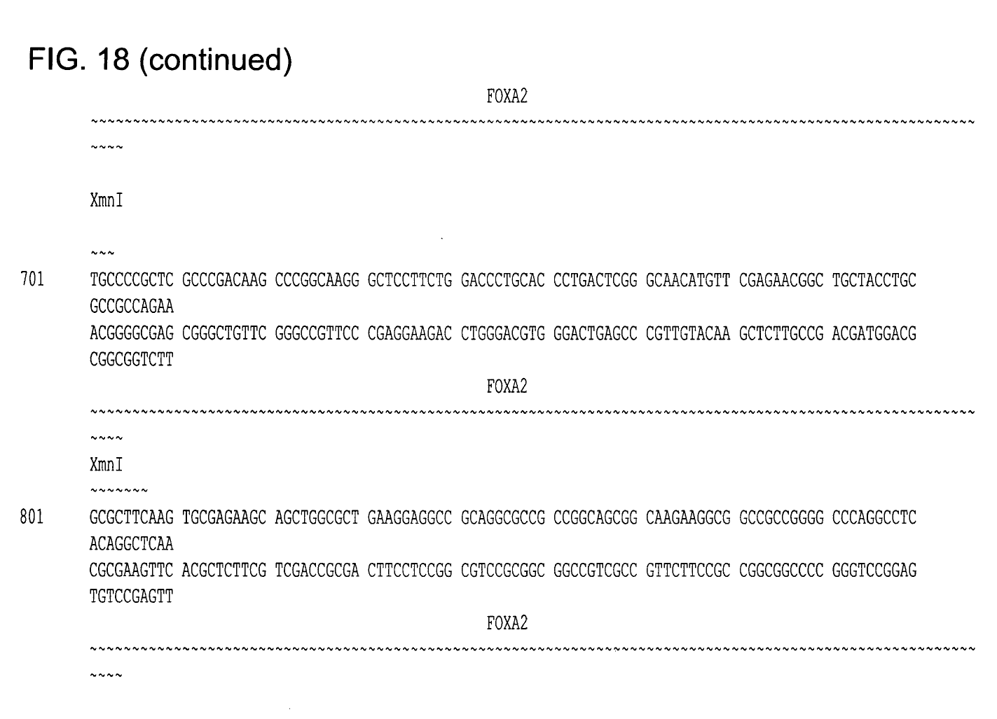

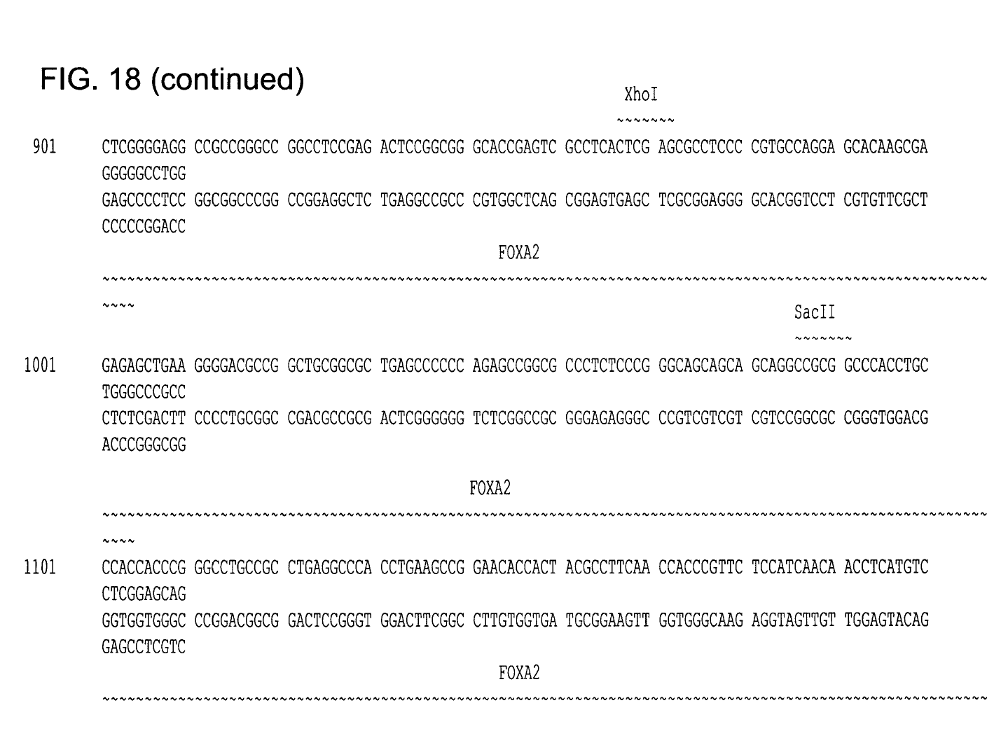

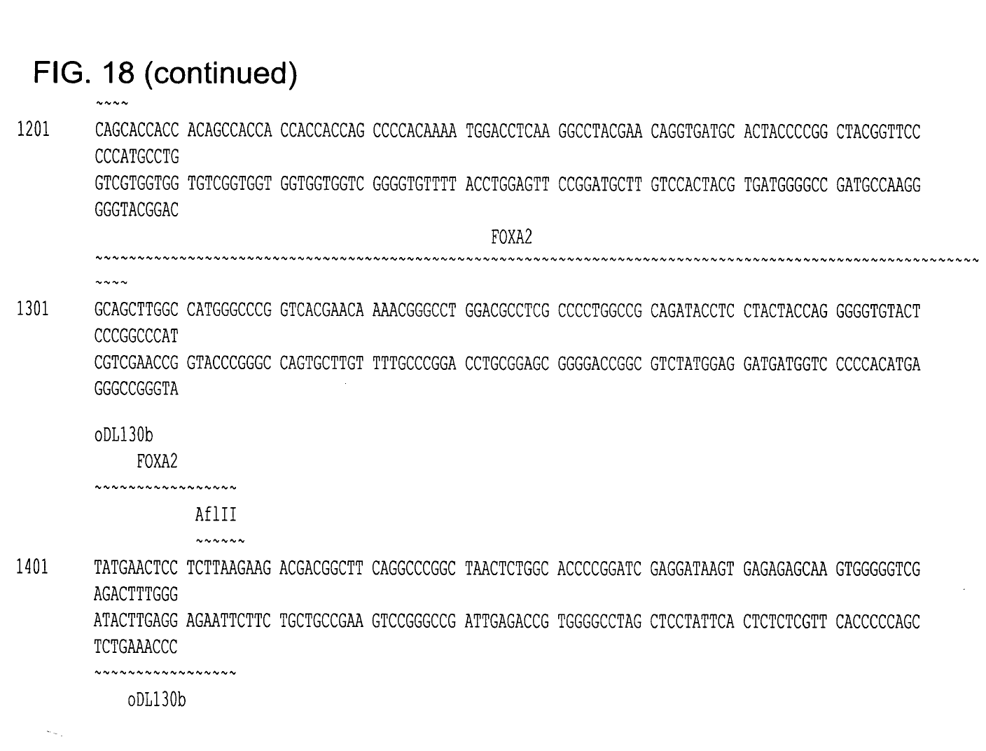

One ordinary skilled artisan in the art will be able to clone the nucleic acids encoding HNF-4-α, HNF-1α, HNF-3β, and CEBP/α and construct expression plasmids for introducing HNF4-α, HNF-1α, HNF-3β, and/or CEBP/α into the stem cells. One exemplary strategy and related information is provided at FIGS. 9-18. For example, conventional polymerase chain reaction (PCR) cloning and molecular techniques can be used to clone the nucleic acids encoding HNF-4-α, HNF-1-α, HNF-3β, and CEBP/α. The nucleic acids can be cloned into a general purpose cloning vector such as pUC19, pBR322, pBluescript vectors (Stratagene Inc.) or pCR TOPO® from Invitrogen Inc. The nucleic acids can be PCR cloned into a vector using the TOPO® cloning method in Invitrogen topoisomerase-assisted TA vectors such as pCR®-TOPO, pCR®-Blunt II-TOPO, pENTR/D-TOPO®, and pENTR/SD/D-TOPO®. Both pENTR/D-TOPO®, and pENTR/SD/D-TOPO® are directional TOPO entry vectors which allow the cloning of the DNA sequence in the 5′→3′ orientation into a Gateway® expression vector (Destination (DEST) vectors, see 9-15). Directional cloning in the 5′→3′ orientation facilitates the unidirection insertion of the DNA sequence into a protein expression vector such that the promoter is upstream of the 5′ ATG start codon of the nucleic acid, enabling promoter driven protein expression. The recombinant vector carrying the nucleic acid of interest can be transfected into and propagated in general cloning E. coli such as XL1Blue, SURE (Stratagene) and TOP-10 cells (Invitrogen). Alternatively, cloning can be done into a an expression vector such as or the strong CMV promoter-based pcDNA3.1 (Invitrogen) and pCIneo vectors (Promega) for mammalian expression; replication incompetent adenoviral vector vectors pAdeno X, pAd5F35, pLP-Adeno-X-CMV (Clontech), pAd/CMV/V5-DEST, pAd-DEST vector (Invitrogen) for adenovirus-mediated gene transfer and expression in mammalian cells; pLNCX2, pLXSN, and pLAPSN retrovirus vectors for use with the Retro-X™ system from Clontech for retroviral-mediated gene transfer and expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells; adenovirus-associated virus expression vectors such as pAAV-MCS, pAAV-IRES-hrGFP, and pAAV-RC vector (Stratagene) for adeno-associated virus-mediated gene transfer and expression in mammalian cells. Further examples include but limited to tetracycline-regulated replication-incompetent herpes simplex virus vectors described in F. Schmeisser, et al. (Human Gene Therapy, 2002, 13: 2113-2124) and the LTRCMVR2, LTRAutoR2, TRECMVR2 and TREAutoR2 lentiviral vectors described in D. Markusic et. al. (Nucleic Acids Research 2005 33(6):e63).

Each PCR primer should have at least 15 nucleotides overlapping with its corresponding templates at the region to be amplified. The polymerase used in the PCR amplification should have high fidelity such as Strategene's PfuUltra™ polymerase for reducing sequence mistakes during the PCR amplification process. For ease of ligating the PCR amplified coding sequence to the designated vector, the PCR primers should also have distinct and unique restriction digestion sites on their flanking ends that do not anneal to the DNA template during PCR amplification. The choice of the restriction digestion sites for each pair of specific primers should be such that the coding nucleic acids are is in-frame and will encode the HNF4-α, HNF-1-α, HNF-3β, and CEBP/α proteins respectively from beginning to end with no stop codons. At the same time the chosen restriction digestion sites should not be found within the SEQ. ID. Nos.: 1-10.

In some embodiments, the expressions of HNF4-α, HNF-1-α, HNF-3β, an/or CEBP/α proteins in the transduced stem cells are constitutive. In other embodiments, the expressions of HNF4-α, HNF-1-α, HNF-3β, an/or CEBP/α proteins in the transduced stem cells are inducible with inducers such as tetracycline or doxycycline.

In some embodiments, the expression vectors used for introducing exogenous HNF4-α, HNF-1-α, HNF-3β, an/or CEBP/α into stem cells include not limited to adenovirus, retrovirus, lentivirus, adeno associated virus, envelope protein pseudotype virus (chimeric virus), herpes simplex virus vectors and virosomes (e.g. liposomes combined with an inactivated HIV or influenza virus).

A simplified system for generating recombinant adenoviruses is presented by He T C. et. al. Proc. Natl. Acad. Sci. USA 95:2509-2514, 1998. The gene of interest is first cloned into a shuttle vector, e.g. pAdTrack-CMV. The resultant plasmid is linearized by digesting with restriction endonuclease Pme I, and subsequently cotransformed into E. coli. BJ5183 cells with an adenoviral backbone plasmid, e.g. pAdEasy-1 of Stratagene's AdEasy™ Adenoviral Vector System. Recombinant adenovirus vectors are selected for kanamycin resistance, and recombination confirmed by restriction endonuclease analyses. Finally, the linearized recombinant plasmid is transfected into adenovirus packaging cell lines, for example HEK 293 cells (E1-transformed human embryonic kidney cells) or 911 (E1-transformed human embryonic retinal cells) (Human Gene Therapy 7:215-222, 1996). Recombinant adenovirus are generated within the HEK 293 cells.

Recombinant lentivirus has the advantage of gene delivery into either dividing and non-dividing mammalian cells. The HIV-1 based lentivirus can effectively transduce a broader host range than the Moloney Leukemia Virus (MoMLV)-base retroviral systems. Preparation of the recombinant lentivirus can be achieved using the pLenti4/V5-DEST™, pLenti6/V5-DEST™ or pLenti vectors together with ViraPower™ Lentiviral Expression systems from Invitrogen.

Recombinant adeno-associated virus (rAAV) vectors are applicable to a wide range of host cells including many different human and non-human cell lines or tissues. Because AAV is non-pathogenic and does not elicit an immune response, a multitude of pre-clinical studies have reported excellent safety profiles. rAAVs are capable of transducing a broad range of cell types and transduction is not dependent on active host cell division. High titers, >10⁸ viral particle/ml, are easily obtained in the supernatant and 10¹¹-10¹² viral particle/ml with further concentration. The transgene is integrated into the host genome so expression is long term and stable.

The use of alternative AAV serotypes other than AAV-2 (Davidson et al (2000), PNAS 97(7)3428-32; Passini et al (2003), J. Virol 77(12):7034-40) have demonstrated different cell tropisms and increased transduction capabilities. With respect to brain cancers, the development of novel injection techniques into the brain, specifically convection enhanced delivery (CED; Bobo et al (1994), PNAS 91(6):2076-80; Nguyen et al (2001), Neuroreport 12(9):1961-4), has significantly enhanced the ability to transduce large areas of the brain with an AAV vector.

Large scale preparation of AAV vectors is made by a three-plasmid cotransfection of a packaging cell line: AAV vector carrying the coding nucleic acid, AAV RC vector containing AAV rep and cap genes, and adenovirus helper plasmid pDF6, into 50×150 mm plates of subconfluent 293 cells. Cells are harvested three days after transfection, and viruses are released by three freeze-thaw cycles or by sonication.

AAV vectors are then purified by two different methods depending on the serotype of the vector. AAV2 vector is purified by the single-step gravity-flow column purification method based on its affinity for heparin (Auricchio, A., et. al., 2001, Human Gene therapy 12; 71-6; Summerford, C. and R. Samulski, 1998, J. Virol. 72:1438-45; Summerford, C. and R. Samulski, 1999, Nat. Med. 5: 587-88). AAV2/1 and AAV2/5 vectors are currently purified by three sequential CsCl gradients.

### Hepatocyte Differentiation Protocol for Transduced Stem Cells.

Generally, the cells are cultured in culture medium, which is a nutrient-rich buffered aqueous solution capable of sustaining cell growth. Culture media suitable for isolating, expanding and differentiating transduced stem cells according to the method described herein include but not limited to high glucose Dulbecco's Modified Eagle's Medium (DMEM), DMEM/F-15, Liebovitz L-15, RPMI 1640, Iscove's modified Dubelcco's media (IMDM), and Opti-MEM SFM (Invitrogen Inc.). Chemically Defined Medium comprises a minimum essential medium such as Iscove's Modified Dulbecco's Medium (IMDM) (Gibco), supplemented with human serum albumin, human Ex Cyte lipoprotein, transferrin, insulin, vitamins, essential and non essential amino acids, sodium pyruvate, glutamine and a mitogen is also suitable. As used herein, a mitogen refers to an agent that stimulates cell division of a cell. An agent can be a chemical, usually some form of a protein that encourages a cell to commence cell division, triggering mitosis. In one embodiment, serum free media such as those described in U.S. Ser. No. 08/464,599 and WO96/39487, and the “complete media” as described in U.S. Pat. No. 5,486,359 are contemplated for use with the method described herein.

In some embodiments, the culture medium is supplemented with 3700 mg/l of sodium bicarbonate and 10 ml/l of a 100× (100 times concentrated) antibiotic-antimycotic cocktail containing 10,000 units of penicillin, 10,000 μg of streptomycin, and 25 μg of amphotericin B/ml utilizing penicillin G (sodium salt), streptomycin sulfate, and amphotericin B (Fungizone™) in 0.85% saline.

In some embodiments, the culture medium is supplemented with 10% Fetal Bovine Serum (FBS), human autologous serum, human AB serum or platelet rich plasma supplemented with heparin (2 U/ml).

Cell cultures are maintained in a CO₂ atmosphere, e.g., 5% to 12%, to maintain pH of the culture fluid, incubated at 37° C. in a humid atmosphere and passaged to maintain a confluence below 85%.

In one embodiment, the transduced stem cells described in the method herein are embedded in a collagen sandwich prior to induction of the differentiation protocol. Following expansion, cells were plated in various collagen sandwich culture configurations as described by Dunn et al. (FASEB J. 1989, 3:174-7; Biotechnol Prog. 1991, 7:237-45; J. Cell Biol. 1992, 116:1043-53; Biotechnol Bioeng. 1993, 41:593-8). Rat tail Type I collagen in 20 mM Acetic Acid is diluted with culture media and pH adjusted to be within a range of 7.4 to 7.9. A first layer of collagen hydrogel is plated in an empty vessel. Enough liquid volume of the collagen is added to achieve a hydrogel base layer thickness of ˜150 μm. The liquid gel is allowed to set for 30 minutes at 37° C. in 5% CO₂ atmosphere. Transduced stem cells are then plated at a density of ˜35,000 cells per cm² on the first collagen layer. Transduced stem cells are allowed to attach overnight. The next day, the media is removed and the cells are gently washed to remove cellular debris before a second collagen hydrogel layer is applied to the top of the cells. The second hydrogel layer is allowed to set at 30 minutes at 37° C. in 5% CO₂ atmosphere. At this time, inducers such as doxycycline-containing media can be added to the cells and the cells are incubated for 24 hours.

In some embodiments, the other hydrogel materials such as gelatin, fibrinogen, chitosan, hylauronic acid, alginate, poly-ethyleneglycol, lactic acid, and N-isopropyl arcrylamide can be used for embedding the transduced stem cells.

Following 24 hours of incubation in inducer-containing media such as doxycycline-containing media, transduced stem cells are differentiated in serum-free Dox+DMEM containing 20 ηg/ml of Epidermal Growth Factor (EGF) and 10 ηg/ml of basic Fibroblast Growth Factor (bFGF) (anti-Pro/DMEM) for 48 hours. Transduced stem cells can be incubated in anti-Pro/DMEM media for up to 7 days. The anti-Pro/DMEM is removed and the cells are liberally washed with PBS. Next, the cells ware cultured with serum-free Dox+DMEM containing 20ηg/ml of Hepatocyte Growth Factor (HGF); 10 ηg/ml of basic Fibroblast Growth Factor (bFGF); and 4.9 mM of nicotinamide (diff/DMEM) for 72 hours. Here again, the cells can be incubated in diff/DMEM media for up to 7 days. Following diff/DMEM incubation, the cells are washed liberally with PBS and maintained in culture for the remainder of the culture period in Dox+Hepatocyte Culture Media (Lonza, St. Louis, Mo.) containing 20 ηg/ml Oncostatin M (Polar/HGM). Cells are maintained in this media for up to 180 days.

### Markers Indicative of a Hepatocyte-Phenotype

The liver functions in carbohydrate, protein, and lipid metabolism. Hepatocyte-like cells generated according to the methods described herein should express characteristic markers indicative of liver function. For example, cells generated according to the methods described herein are expected to express enzymes and other polypeptides associated with carbohydrate, protein, and lipid metabolism. In one embodiment, a cell of the invention expresses a polypeptide associated with glycogen storage, glucose-6-phosphatase activity, decomposition of red blood cells, or plasma protein synthesis. In other embodiments, a cell of the invention expresses a polypeptide associated with urea production or synthesis of bile. In one embodiment, the cell expresses a polypeptide associated with cytochrome p450 (CYP3A4) activity, which is responsible for xenobiotic detoxification. In other embodiments, the cell expresses arginase I, which functions in physiologic detoxification and urea production. In still other embodiments, the cell expresses glucose-6-phosphatase.

The expression of a hepatocyte phenotype in a cell of the invention may be evaluated by analysing mRNA syntheses. In some embodiments, the mRNAs of key enzymes and proteins expressed in the hepatocyte-like cell are evaluated by quantitative reverse transcriptase polymerase chain reaction (qRT-PCR). In other embodiments, the mRNAs encoding glucose-6-phosphatase, CYP3A4 and arginase I are determined by quantitative RT-PCR using custom primer pairs for the proteins.

Reverse Transcriptase PCR is an amplification technique that can be used to determine levels of mRNA expression. (See, e.g., Gibson et al., Genome Research 6:995-1001, 1996; Heid et al., Genome Research 6:986-994, 1996). Reverse Transcriptase PCR evaluates the level of PCR product accumulation during amplification. This technique permits quantitative evaluation of mRNA levels in multiple samples. For mRNA levels, mRNA is extracted from a biological sample, e.g. a tumor and normal tissue, and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes can be obtained commercially from, for example, Perkin Elmer/Applied Biosystems (Foster City, Calif.). To quantify the amount of the specific nucleic acid of interest in a sample, a standard curve is generated using a control. Standard curves can be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

The TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification (see, for example, www2.perkin-elmer.com).

One of ordinary skill in the art should be able to design PCR primers for amplifying the mRNAs of the genes described herein. In one embodiment, the PCR primers are derived from SEQ. ID. Nos. 1-10.

Methods of “quantitative” amplification are well known to those of skill in the art. For example, quantitative PCR involves simultaneously co-amplifying a known quantity of a control sequence using the same primers. This provides an internal standard that can be used to calibrate the PCR reaction. Detailed protocols for quantitative PCR are provided, for example, in Innis et al. (1990) PCR Protocols, A Guide to Methods and Applications, Academic Press, Inc. N.Y.

In other embodiments, cells of the invention (e.g., hepatocyte-like cells) are characterized by evaluating enzymatic activity. Such assays may be carried out on intact or homogenized cells.

In one embodiment, glucose-6-phosphatase activity is assayed. Liver glucose-6 phosphatase (EC 3.1.3.9) is a key enzyme in glucose production, catalyzing the terminal step of both gluconeogenic and glycogenolytic pathways. The enzyme catalyses the dephosphorylation of glucose-6-phosphate to give glucose and inorganic phosphate.

The standard glucose-6-phosphatase assay is performed at 30° C. and pH 7.3 in a medium composed of 20 mM Tris-HCl and 20 mM glucose-6 phosphate. A clarified cell lysate is prepared by methods known to one skilled in the art. The lysate is added to initiate the enzyme reaction. After 10 minutes of incubation, 10% ice-cold trichloroacetic acid is added to terminate enzyme reaction. The inorganic phosphate produced as the by-product of the enzyme activity is determined according to Baginski et al. (In: Methods of Enzymatic Analysis (Bergmeyer, H. U., ed.), vol. 2, pp. 876-880. Verlag Chemie International, Deerfield Beach, Fla. 1974). Controls are run in the absence of enzyme and/or substrate.

Another glucose-6-phosphatase assay using malachite green detection method is described by Petrolonis et. al. (J. Biol. Chem., 2004, 279:13976-13983). A concentration of 0.38 mg/ml of protein preparation is used for each 20-μl assay. Protein amounts are kept constant and monitored using the Pierce BCA Protein Plus assay. 4 μl of lysate and 16 μl of assay buffer containing 0.62 mM glucose-6-phosphate, 62 mM MES K+ pH 6.5 are added to each well to give final concentrations of 0.5 mM G-6-P, 50 mM MES pH 6.5. The reaction is incubated for 40 minutes and quenched using 80 μl of the detection reagent with 0.067% Tween 20. Then, the reaction is incubated at ambient temperature for 35 minutes for color development. Absorbance was measured at 650 nm using the Molecular Devices SpectraMax (Sunnyvale, Calif.). Controls are run in the absence of enzyme and/or substrate.

Additional glucose-6-phosphatase assay is described in http://www.sigmaaldrich.com/sigma/enzyme%20assay/gluc6phosphatase.pdf.

In other embodiments, cells of the invention are assayed for activity indicative of xenobiotic detoxification. Detoxification by CYP3A4 is demonstrated using the P450-Glo™ CYP3A4 DMSO-tolerance assay (Luciferin-PPXE) and the P450-Glo™ CYP3A4 cell-based/biochemical assay (Luciferin-PFBE) (Promega Inc., # V8911 and # V8901). Detoxification by CYP1A1 and or CYP1B1 is demonstrated using the P450-Glo™ assay (Luciferin-CEE) (Promega Inc., # V8762). Detoxification by CYP1A2 and or CYP4A is demonstrated using the P450-Glo™ assay (Luciferin-ME) (Promega Inc., # V8772) Detoxification by CYP2C9 is demonstrated using the P450-Glo™ CYP2C9 assay (Luciferin-H) (Promega Inc., # V8791).

Arginase I (EC 3.5.3.1) catalyses the deamination of L-arginine to L-ornithine and urea. The standard arginase assay is performed at 37° C. and pH 7.0 in a medium composed of 50 mM manganese maleate activation buffer and 713 mM L-arginine. The clarified cell lysate is incubated in 50 mM manganese maleate activation buffer for 4 h at 37° C. and diluted in the same buffer before use. The enzyme reaction is initiated with L-arginine incubated for 37° C. for 30 minutes. Bun Acid color reagent (BUN, Sigma #535-A, urea nitrogen kit from Sigma) is added and the production of urea is measured colometrically at 535 nm. Detailed arginase assay can be found at http://www.sigmaaldrich.comimg/assets/18200/Arginase.pdf. Other urea colorimetric assay kits, such as QuantiChrom from BioAssay Systems, can be used.

Alternatively, hepatocyte-like cells are characterized for a hepatocyte phenotype by analysing the expression of hepatocyte markers (e.g., polypeptides characteristically expressed in hepatocytes. In one embodiment, protein expression is assayed in an immunoassay, such as an immunocytochemical assay or a Western blot.

In one embodiment, hepatocyte-like cell are harvested and lysed using any method known in the art. Methods of lysing the cells are featured in “Sample Preparation-Tools for Protein Research” EMD Bioscience and in the Current Protocols in Protein Sciences (CPPS) and they are incorporated hereby reference in their entirety. Lysates of hepatocyte-like cells are then clarified by centrifugation. The soluble proteins in the clarified lysates are separated by SDS-PAGE and transferred to nitrocellulose membrane (or other suitable substrate) for immunostaining with appropriate antibodies for the respective expressed proteins. Methods of SDS-PAGE, Western blot and immunostaining can be found in Current Protocols in Protein Sciences (CPPS). Examples of some of antibodies useful for characterizing a hepatocyte phenotype are polyclonal antibodies against CYP450 3A4 (Oxford Biomedical Research, # PA32), against alpha 1 fetoprotein (Abcam® catalog #ab33992), against glucose-6-phosphatas-alpha (C-14), (H-60) and (N-19) (Santa Cruz Biotechnology); against human serum albumin (Abcam® catalog #ab2406); and against arginase I (8C9) (Santa Cruz Biotechnology, catalog #sc-47715).

In another approach, protein synthesis is evaluated in an ELISA. For example, the secretion of human albumin and α-fetoalbumin is determined by analysing the culture media of a cell of the invention by ELISA. The hepatocyte-like cell are rinsed with phosphate buffered saline (PBS) three times and incubated with serum-free hepatocyte growth media at 37° C. for 3 hours. The spent culture media is collected and analyzed. Human albumin can be determined using commercially available human albumin ELISA kits from Bethyl Laboratories (catalog #E80-129), Immundiagnostik (catalog #K 6330), and Immuno-Biological Laboratories, Inc. (catalog #ORG 5MA).

Alpha 1 Fetoprotein is a major plasma protein produced by the yolk sac and the liver during fetal life. Fully differentiated hepatocytes do not express α-fetoalbumin. Alpha 1 Fetoprotein can be determined using commercially available human α-fetoalbumin ELISA kits from Abnova Corporation (catalog #KA0049), Calbiotech, Inc (catalog #AF064T), and Diagnostic Systems Laboratories, Inc. (catalog #DSL-10-8400).

In another approach, the biological function of a hepatocyte-like cell is evaluated, for example, by analysing glycogen storage. Glycogen storage is characterized by assaying Periodic Acid Schiff (PAS) functional staining for glycogen granules. The hepatocyte-like cells are first oxidized by periodic acid. The oxidative process results in the formation of aldehyde groupings through carbon-to-carbon bond cleavage. Free hydroxyl groups should be present for oxidation to take place. Oxidation is completed when it reaches the aldehyde stage. The aldehyde groups are detected by the Schiff reagent. A colorless, unstable dialdehyde compound is formed and then transformed to the colored final product by restoration of the quinoid chromophoric grouping (Thompson S W. in Selected Histochemical and Histopathological Methods, C. C. Tomas, Sprungfield, Ill., 1966; Sheehan D C. and Hrapchak, B B. in Theory and Practise of Histotechnology, 2nd Ed., Battelle memorial Institute, Columbus, Ohio, 1987). PAS staining can be performed according the protocol described at http://www.jhu.edu/˜iic/PDF_protocols/LM/Glycogen_Staining pdf and http://library.med.utah.edu/WebPath/HISTHTML/MANUALS/PAS.PDF with some modifications for an in vitro culture of hepatocyte-like cells. One of ordinary skill in the art should be able to make the appropriate modifications.

In another approach, a hepatocyte-like cell of the invention is characterized for urea production. Urea production can be assayed colorimetrically using kits from Sigma Diagnostic (Miyoshi et al., 1998, J Biomater Sci Polym Ed 9: 227-237) based on the biochemical reaction of urease reduction to urea and ammonia and the subsequent reaction with 2-oxoglutarate to form glutamate and NAD.

In another approach, bile secretion is analysed. Biliary secretion can be determined by fluorescein diacetate time lapse assay. Briefly, monolayer cultures of hepatocyte-like cells are rinsed with phosphate buffered saline (PBS) three times and incubated with serum-free hepatocyte growth media supplemented with doxycycline and fluorescein diacetate (20 μg/ml) (Sigma-Aldrich) at 37° C. for 35 minutes. The cells are washed with PBS three times and fluorescence imaging is carried out. Fluorescein diacetate is a non fluorescent precursor of fluorescein. The image is evaluated to determine that the compound had been taken up and metabolized in the hepatocyte-like cell to fluorescein. In some embodiments, the compound is secreted into intercellular clefts of the monolayer of cells. Alternatively, bile secretion is determined by a method using sodium fluorescein described by Gebhart B. and Wang L. (J. Cell Sci. 1982, 56233-244).

In yet another approach lipid synthesis is analysed. Lipid synthesis in the hepatocyte-like cell can be determined by oil red O staining Oil Red O (Solvent Red 27, Sudan Red 5B, C.I. 26125, C26H24N4O) is a lysochrome (fat-soluble dye) diazo dye used for staining of neutral triglycerides and lipids on frozen sections and some lipoproteins on paraffin sections. It has the appearance of a red powder with maximum absorption at 518(359) nm. Oil Red O is one of the dyes used for Sudan staining. Similar dyes include Sudan III, Sudan IV, and Sudan Black B. The staining has to be performed on fresh samples and/or formalin fixed samples. Hepatocyte-like cells are cultured on microscope slides, rinsed in PBS three times, the slides are air dried for 30-60 minutes at room temperature, fixed in ice cold 10% formalin for 5-10 minutes, and then rinse immediately in 3 changes of distilled water. The slide is then placed in absolute propylene glycol for 2-5 minutes to avoid carrying water into Oil Red O and stained in pre-warmed Oil Red O solution for 8 minutes in 60° C. oven. The slide is then placed in 85% propylene glycol solution for 2-5 minutes and rinsed in 2 changes of distilled water. Oil red O staining can also be performed according the protocol described at library.med.utah.edu/WebPath/HISTHTML/MANUALS/OILRED.PDF with some modifications for an in vitro culture of hepatocyte-like cell by one of ordinary skill in the art.

In still another approach, the cells are assayed for glycogen synthesis. Glycogen assays are well known to one of ordinary skill in the art, for example, in Passonneau and Lauderdale, 1974, Anal. Biochem., 60:405-415. Alternatively, commercial glycogen assays can be used, for example, from BioVision, Inc. catalog #K646-100.

The present invention provides methods of treating disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a liver disease or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which a reduction in liver function may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with a reduction in liver function, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Uses of Hepatocyte-Like Cell

The hepatocyte-like cell of the invention can be used in a variety of applications. These include but not limited to transplantation or implantation of the hepatocytes in vivo; screening cytotoxic compounds, carcinogens, mutagens growth/regulatory factors, pharmaceutical compounds, etc., in vitro; elucidating the mechanism of liver diseases and infections; studying the mechanism by which drugs and/or growth factors operate; diagnosing and monitoring cancer in a patient; gene therapy; and the production of biologically active products, to name but a few.

Fulminant hepatic failure (FHF) is a clinical syndrome defined by acute liver injury leading to hepatic encephalopathy within 8 weeks of the onset of jaundice. Large amount of toxic metabolic by-products such as ammonia accumulates in the blood. The only successful treatment for FHF, when poor prognostic signs are present, is emergent orthotropic liver transplantation (Sass and Shakil, 2003, Gastroenterol. Clin North Am 32: 1195-1211). There is always a shortage of suitable organs and often a liver is not available soon enough to prevent death. Other treatment modalities have been developed to support the patient with FHF until a suitable liver allograft is obtained for transplantation or the patient's own liver regenerates sufficiently to resume normal functions. These include extracorporeal devices (Selden and Hodgson, 2004, Transpl Immunol 12: 273-288), cell transplantation (Selden and Hodgson, 2004, supra), and tissue-engineered constructs (Dvir-Ginzberg et al., 2003, Tissue Eng 9: 757-66).

In one embodiment, provided herein is a method of neutralizing a toxic compound in a bodily fluid of a mammal, the comprising contacting the bodily fluid with a hepatocyte-like cell described herein, wherein the contacting step takes place in a perfusion device, for example a bio-artificial liver.

The hepatocyte-like cell cultures described herein can be used the construction of extracorporeal liver assist device such as a bio-artificial liver for use by subjects having liver disorders that result in hepatic failure or insufficiency. The use of such bio-artificial livers involves the perfusion of the subject's blood through the bio-artificial liver. In the blood perfusion protocol, the subject's blood is withdrawn and passed into contact with the hepatocyte-like cell cultures. During such passage, molecules dissolved in the patient's blood, such as bilirubin, are taken up and metabolized by the hepatocyte cultures. In addition, the hepatocyte-like cell provide factors normally supplied by liver tissue.

To form the bio-artificial liver the hepatocyte-like cells of the invention are cultured within a containment vessel containing an input and output outlet for passage of the subject's blood through the containment vessel. The bio-artificial liver further includes a blood input line which is operatively coupled to a conventional peristaltic pump. A blood output line is also included. Input and output lines are connected to appropriate arterial-venous fistulas which are implanted into, for example, the forearm of a subject. In addition, the containment vessel may contain input and output outlets for circulation of appropriate growth medium to the hepatocytes for continuous cell culture within the containment vessel.

In an embodiment, semipermeable membranes can be included in the bio-artificial livers to prevent direct contact of the subject's blood with the three-dimensional hepatocyte cultures. In such instances, the molecules dissolved in the subject's blood will diffuse through the semipermeable membrane and are taken up and metabolized by the hepatocycte cultures.

In some embodiments, the hepatocyte-like cells are used to seed the bioartificial liver systems such as those described in U.S. Pat. Nos. 5,605,835, 5,955,353, 7,160,719, and WO/2003/104411, and in Wei-Shou Hu, et. al., (Cytotechnology, 2004, 23: 29-38).

The hepatocyte-like cell cultures of the invention can be used for the construction of a tissue engineered liver. A tissue engineered liver can provide a new therapy in which the differentiated hepatocytes are transplanted within three-dimensional polymer scaffolds to supplement or replace the function of a failing liver.

A tissue engineered liver can be made of hepatocyte-like cell fabricated onto a matrix or a scaffold made of natural or manmade material. In one embodiment, the differentiated hepatocytes can be used to seed a decellularized liver scaffold as described in U.S. Patent Application 20050249816. Manmade materials that can be used are often biodegradable polymers, such as the three-dimensional tissue culture system in which stromal cells were laid over a polymer support system (See U.S. Pat. No. 5,863,531). Materials suitable for polymer scaffold fabrication include polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly (alpha-hydroxy acid), polycaprolactone, polycarbonates, polyamides, polyanhydrides, polyamino acids, polyorthoesters, polyacetals, polycyanoacrylates, degradable urethanes, aliphatic polyesterspolyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, Teflon®, nylon silicon, and shape memory materials, such as poly (styrene-block-butadiene), polynorbornene, hydrogels, metallic alloys, and oligo (s-caprolactone) diol as switching segment/oligo (p-dioxyanone) diol as physical crosslink. Other suitable polymers can be obtained by reference to The Polymer Handbook, 3rd edition (Wiley, N.Y., 1989).

In one embodiment, the construction of a three-dimensional polymer-cell scaffold made of polymer and hepatocyte-like cell is performed according to WO/2003/076564 and U.S. Pat. Nos. 5,624,840 and 5,759,830. Such tissue engineered liver can be implanted into the patient to restore liver function.

In one embodiment, the hepatocyte-like cell can also be administered or transplanted to the recipient in an effective amount to achieve restoration of liver function, thereby alleviating the symptoms associated with liver disorders.

The number of cells needed to achieve the purposes of restoring liver function, either fully or partially, will vary depending on the degree of liver damage and the size, age and weight of the host. For example, the cells are administered in an amount effective to restore liver functions. Determination of effective amounts is well within the capability of those skilled in the art. The effective dose can be determined by using a variety of different assays designed to detect restoration of liver function. The progress of the transplant of the recipient can be determined using assays that include blood tests known as liver function tests. Such liver function tests include assays for alkaline phosphatase, alanine transaminase, aspartate transaminase and bilirubin. In addition, recipients can be examined for the presence or disappearance of features normally associated with liver disease such as, for example, jaundice, anemia, leukopenia, thrombocytopenia, increased heart rate, and high levels of insulin. Further, imaging tests such as ultrasound, computer assisted tomography (CAT) and magnetic resonance (MR) may be used to assay for liver function.

The hepatocyte-like cell can be administered by conventional techniques such as injection of cells into the recipient host liver, injection into the portal vein, or surgical transplantation of cells into the recipient host liver. In some instances it can be necessary to administer the hepatocyte-like cell more than once to restore liver function. In addition, growth factors, such as G-CSF, or hormones, and TGF-β1 can be administered to the recipient prior to and following transplantation for the purpose of priming the recipient's liver and blood to accept the transplanted cells and/or to generate an environment supportive of hepatic cell proliferation.

In one embodiment, the hepatocyte-like cell can be used for the production of blood coagulation factors. These coagulation factors are useful for subjects with hemophilia and other blood clotting disorders. After cultured hepatocyte-like cells have reached confluency, the supernatant culture media can be collected and purified according to methods known in the art, such as those described in U.S. Pat. No. 4,789,733 and W H Kane and P W Majerus (J. Biol. Chem., 256:1002-1007, 1981). Currently, most of the preparations of blood coagulation factors are from donated blood and that presents the disadvantage that the danger of transmitting hepatitis. However, manufacturing blood coagulation factors from the hepatocyte-like cell described herein greatly reduces the risk of transmitting hepatitis or other blood borne diseases.

In one embodiment, the hepatocyte-like cell can be used for in vitro screening for toxicity of a wide variety of compounds, such as cytotoxic compounds, growth/regulatory factors, pharmaceutical agents, etc. in order to identify those that are most efficacious; i.e. those that kill the malignant or diseased cells, yet spare the normal cells. To this end, cultures of hepatocyte-like cell are maintained in vitro and exposed to the compound to be tested. The activity of a cytotoxic compound can be measured by its ability to damage or kill cells in culture. This can readily be assessed by vital staining techniques that are known to one skilled in the art. A simple method is set forth herein. After exposure to the test compound for a fix period, the cells are rinsed with PBS, and incubated for 40 min in a solution of 5 μL of calcein AM and 20 μL of ethidium homodimer-1 in 10 mL of DPBS (dead cells will fluoresce red, and live cells fluoresce green). Cellular fluorescence can be observed in an inverted epifluorescent microscope (Olympus USA, Melville, N.Y.) using a FITC/RhoA band filters. In one embodiment, the hepatocyte-like cell are used in cytotoxicity screens as described in U.S. Pat. No. 7,041,501.

The effect of growth/regulatory factors can be assessed by analyzing the cellular content of the matrix, e.g., by total cell counts, and differential cell counts. This can be accomplished using standard cytological and/or histological techniques that are known to one skilled in the art including the use of immunocytochemical techniques employing antibodies that define type-specific cellular antigens.

In one embodiment, the hepatocyte-like cell can be used for screening for drug compounds. The effect of various drugs on normal cells cultured in the hepatocyte-like cell can be assessed. For example, drugs that affect cholesterol metabolism, by lowering cholesterol production, could be tested on the these hepatocyte-like cells.

It is well known that a number of compounds fail to act as mutagens in test organisms such as bacteria or fungi, yet cause tumors in experimental animals such as mice. This is due to metabolic activation; i.e., some chemicals are metabolically altered by enzymes in the liver (the P450 oxidase system and hydroxylation systems) or other tissues, creating new compounds that are both mutagenic and carcinogenic. In order to identify such carcinogens, Ames and his co-workers devised a screening assay which involves incubating the chemical compound with liver extracts prior to exposure of the test organism to the metabolic product (Ames et al., 1975, Mut. Res. 31:347-364). The hepatocyte-like cell can be used as a substitute for the liver extracts described in the Ames assay. In some embodiments, the hepatocyte-like cells are used in ADME/Tox analysis in high throughput drug screening. In one embodiment, the hepatocyte-like cells are used in drug screens as described in U.S. Pat. No. 7,282,366.

In one embodiment, the hepatocyte-like cells are used as models for hepatitis virus and other liver disease studies. These models can be useful in the development of anti-viral drugs. The hepatocytes of the invention provide a constant source of hepatocytes for researchers. Hepatitis virus-infected hepatocytes can be grown in vitro and researchers can study the progression of the disease and virus life cycle. The virus-infected hepatocytes can also be used to screen for pharmaceutical agents and compounds that can halt the virus life cycle, kill the virus, eradicate the virus from the hepatocytes, but have tolerable cytotoxicity the hepatocytes. Such pharmaceutical agent or compound treated virus-infected hepatocytes can be screened for cell viability, low or no viral particles count and non diminished, non compromised maintenance of hepatocytes metabolic activities such as lipidogenesis, albumin synthesis, P450 activity, and urea production etc. as described herein.

Accordingly, in one embodiment, provided herein is a method of evaluating the toxicity of a compound in vitro, the method comprising: providing a differentiate engineered hepatocyte described herein; contacting the hepatocyte with the compound to generate a cell supernatant; measuring the metabolic activity or viability of the hepatocyte, wherein a decrease in metabolic activity or viability in the presence of the supernatant compared to that in the absence of the supernatant indicates that the compound is toxic in vivo.

In another embodiment, provided herein is a method of evaluating the toxicity of a compound in vitro, comprising: (a) providing a first hepatocyte-like cell described herein; (b) contacting the first hepatocyte with the compound to generate a cell supernatant; (c) removing the cell supernatant from the first hepatocyte; (d) providing a second hepatocyte-like cell described herein; (e) contacting the second hepatocyte of step (d) with the supernatant; and measuring the metabolic activity of the second hepatocyte, wherein a decrease in metabolic activity in the presence of the supernatant compared to that in the absence of the supernatant indicates that the compound is toxic in vivo.

In yet another embodiment, provided herein is a method of evaluating the toxicity of a compound in vitro, the method comprising: (a) providing a first hepatocyte-like cell described herein; (b) contacting the first hepatocyte with the compound to generate a cell supernatant; (c) removing the cell supernatant from the first hepatocyte; (d) providing a second hepatocyte-like cell described herein; (e) contacting the hepatocyte of step (d) with the supernatant; and measuring the viability of the second hepatocyte, wherein a decrease in viability in the presence of the supernatant compared to that in the absence of the supernatant indicates that said compound is toxic in vivo. In some embodiments, the metabolic activity of the second hepatocyte is measured by assessing positive glucose-6-phosphatase, CYP 3A4, alkaline phosphatase, alanine transaminase, aspartate transaminase and/or arginase I enzyme activities, and positive urea, bile, albumin, and lipid syntheses by methods known to one skilled in the art and described herein.

In various embodiments, the invention provides for the treatment or modeling of specific disease conditions from individual patients. Human diseases such as in-born errors of metabolism, glycogen storage diseases, and Hemophilia B (Christmas Disease) will be modeled through the reprogramming of iPS cells from patients with these diseases. These cells will then be an invaluable resource available for researchers in the respective fields to explore therapeutic options. It has been shown that human somatic cells can be directly reprogrammed in vitro into a pluripotent embryonic stem cell-like state by introducing the combination of four transcription factors (OCT4, SOX2, and either cMYC and KLF4 or NANOG and LIN28).

Human embryonic stem cells (hESCs) and induced pluripotent stem cells (hIPs) lines will be generated from fibroblasts and/or keratinocytes from volunteers using a retroviral system and cultured in human embryonic stem cell conditions as previously described. Such lines will be established for normal individuals, patients with genetic liver diseases (Alagille syndrome; Disorders of carbohydrate metabolism (Glycogen storage disease, type IV; Galactosemia; Fructosemia); Disorders of amino acid metabolism (Tyrosinemia); Disorders of glycolipid and lipid metabolism (Niemann-Pick disease, types A and C; Hunter's disease; Hurler's disease; Wolman's disease); Disorders of glycoprotein metabolism (Gaucher's disease); Metal storage disorders (Hemochromatosis; Wilson's Disease); Peroxisomal disorders (Zellweger syndrome; Mitochondrial cytopathies); Hereditary disorders of bilirubin metabolism (Crigler-Najjar syndrome; Gilbert syndrome; Dubin-Johnson syndrome); Hereditary disorders of bile formation (Progressive familial intrahepatic cholestasis); Disorders of bile acid biosynthesis; and Disorders of protein biosynthesis and targeting (α₁-Antitrypsin deficiency; Cystic fibrosis) and individuals with acute liver failure arising from a combination of genetic and environmental factors. The hiPS cell lines generated will be subjected to quality control analyses such as pluripotent marker gene expression, karyotyping, and teratoma formation to confirm pluripotency.

The hESCs and hiPS derived hepatocytes will be compared from normal individuals and patients with genetic liver diseases using a variety of techniques—genome-wide expression profiling, proteomics, metabolic assays, and biliary excretion—to validate the use of these cells as a basis of research for the pathophysiological state of their respective liver disease. Following the completion of these studies, the differentiated cells will be used as an in vitro model for hepatocyte physiology in health and disease. The ability to easily create a renewable source of patient-specific hepatocytes will be an invaluable tool to potentiate high-throughput therapeutic screening, identify hepatotoxicity pharmaceuticals and model metabolic effects of genetic liver disease.

The references cited herein and throughout the specification and example are herein incorporated by reference in their entirety. This invention is further illustrated by the following example which should not be construed as limiting. The contents of all references cited throughout this application, as well as the figures and table are incorporated herein by reference.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### HNF4-α Expression Induced Expression of Hepatocyte Markers in ADMSCs

Using a lenti-viral vector ADMSCs were transduced with doxycycline-inducible plasmids encoding HNF4-α and GFP. Following expansion of the cells they were induced with doxycycline and cultured in Hepatocyte Culture Media (Lonza, Basel, Switzerland) for 96 hours. They were evaluated for the presence of liver-specific indicators of differentiation HNF4-α, CYP 3A4 and ZO-1.

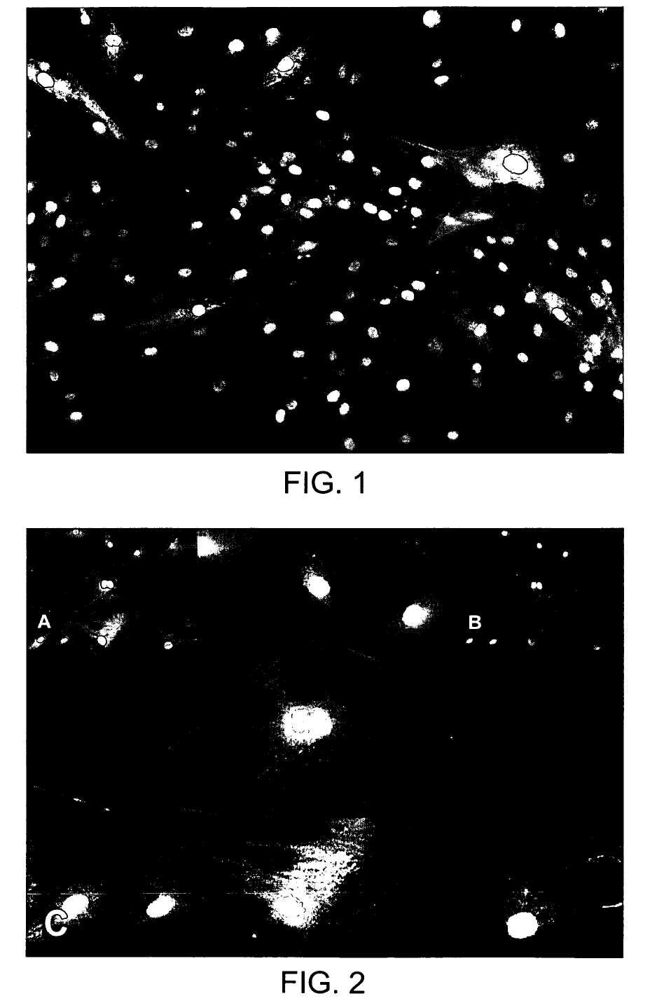

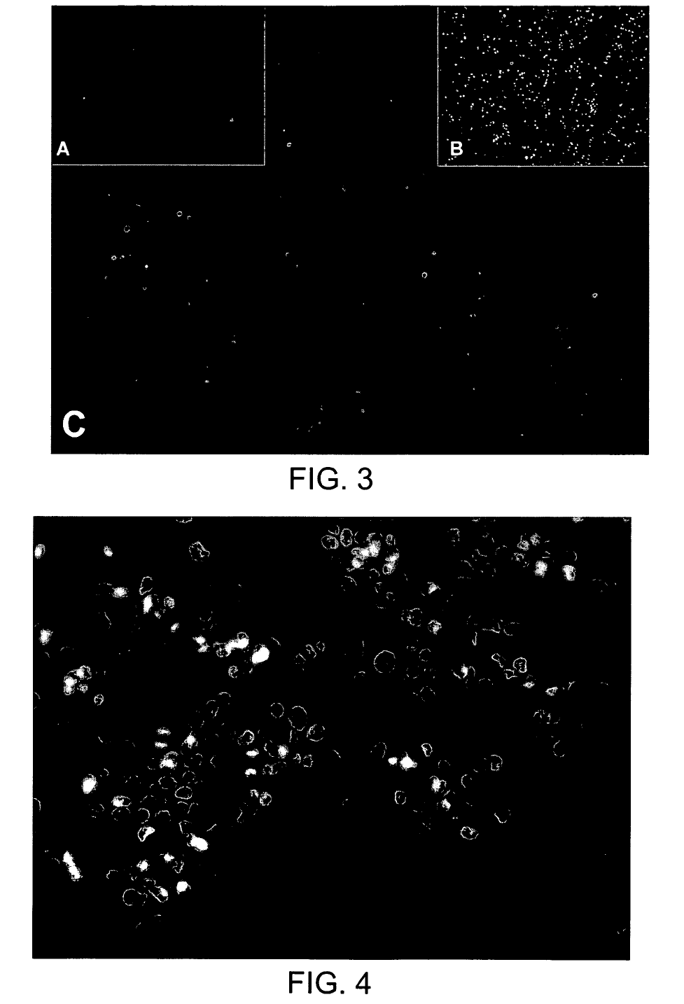

After 60 hours in culture, the cells were evaluated for the presence of HNF4-α by immunohistochemical (IHC) staining (FIG. 1). They were found to be expressing the liver-specific enzyme CYP 3A4 by IHC staining (FIG. 2). Within 7 days, the cells demonstrated tight junction formation by IHC staining for Zona Occludens-1 (FIG. 3).

Since this method of inducing differentiation involves the over-expression of a transcription factor, as opposed to a specific gene of interest, the resulting cells retain the potential to express the donor's native hepatic transcriptome and proteome. Differentiating ADMSCs to hepatocytes in this manner may expand the potential applications of hepatic tissue engineering to include: high-throughput drug screening with individual and population based cellular differences; donor-specific cellular replacement therapy; and eventually donor-derived organ replacements.

### Example 2

### Reprogramming of ADMSCs

Isolation of Adipose-Derived Mesenchymal Stem Cells

Percutaneous lipo-aspirate samples were obtained as surgical waste. The sample was placed in a sterile 1 liter bottle and washed with Phosphate Buffered Saline (PBS) until visibly clear of blood. The PBS was drained and the sample was covered with collagenase solution and incubated on a shaker table for 30 minutes at room temperature. Connective tissue remnants were removed by gravity filtration through a 100 μm centrifuge tube filter. The cellular component of the filtrate was separated by centrifugation at 4000 rpm for 5 minutes. The fat and aspirate fraction are discarded and the pellets are re-suspended with MSC Growth Medium (Dulbecco's Modified Essential Medium (DMEM) containing 10% Fetal Bovine Serum (FBS)). The cells were washed three times and incubated in red blood cell lysis buffer for 15 minutes. The lysis buffer is de-activated with MSC Growth Medium and washed with PBS twice to remove inactivated buffer. The cleaned pellets are re-suspended with MSC Growth Medium and plate in T75 Flasks. The MSC Growth Medium is changed within 12 hours of plating to clear cellular debris and the cells are passaged on day 2. Cells were cultured in MSC Growth Medium at 37° C. in a 5% CO₂ atmosphere and passaged to maintain a confluence below 85%.

### Viral Plasmid Preparation

All Lenti-viral vectors were prepared as described by Stadtfeld et al., 2008b. Briefly a constitutively expressed reverse tetracycline-controlled transactivator (rtTA) viral vector plasmid and tetracycline-inducible viral vectors plasmids of Hepatocyte Nuclear Factor 4a (Dox-HNF4α), and eGFP (Dox-GFP) were constructed and the plasmids were isolated using an Endo-Free Plasmid Purification Maxi-Kit (Qiagen, Valencia, Calif.). Virus was packaged for transduction by transfecting Human Embryonic Kidney 293T (HEK 293) cells at 70% confluence in a 10 cm plate with 10 μg of one viral vector (rtTA, Dox-HNF4α, Dox-eGFP); 6.5 μg pMDL; 2.5 μg pREV; and 3.5 μg VSV-G. Virus was harvested 48 and 72 hours after transfection.

### Transduction of Adipose-Derived Mesenchymal Stem Cells

Cells were expanded until passage four and were kept at less than 85% confluent. Populations of 10⁶ cells were plated on 10 cm dishes and transduced with supernatant of rtTA, Dox-HNF4α, Dox-eGFP. The plates were incubated in virus supernatant for 48 hours. After the transduction incubation period, the cells were washed and maintained in MSC Growth Media for 48 hours in virus quarantine. Following the quarantine period, the cells were re-plated in T-150 plates and expanded.

### Collagen Sandwich Culture

Following expansion, cells were plated in various collagen sandwich culture configurations as described by Dunn et al. Briefly, using Rat Tail Type I Collagen in the following proportions collagen hydrogels were created.

| Proportions for Collagen | | | |
|---|---|---|---|
| Hydrogel | μl | μl | μl |
| Desired Volume | 500 | 1250 | 1500 |
| 10X Phenol-Free DMEM/F12 | 50 | 125 | 150 |
| (1:1) | | | |
| diH20 | 245 | 612.5 | 735 |
| 0.34N NaOH | 2.5 | 6.25 | 7.5 |
| 5 ml/Collagen Type I in | 200 | 600 | 700 |
| 20 mM Acetic Acid | | | |
| Total Volume | 497.5 | 1345 | 1587.5 |

Once mixed, the pH of each gel was measured and corrected to be within a range of 7.4 to 7.9. Collagen hydrogels were plated in vessels in the following liquid volumes to achieve a hydrogel base layer thickness of ˜150 μm.

| | Volume | Total |
|---|---|---|
| Vessel | per well | Volume |
| 3.5 cm | 0.25 ml | 0.25 ml |
| Dish | | |
| 6-well | 0.25 ml | 1.50 ml |
| Plate | | |
| 6 cm | 0.5 ml | 0.5 ml |
| Dish | | |
| 10 cm | 1.25 ml | 1.25 ml |
| Dish | | |
| 24 well | 0.0625 ml | 1.5 ml |
| Plate | | |

The liquid gels were allowed to set for 30 minutes at 37° C. in 5% CO₂ atmosphere. Transduced ADMSCs were plated at a density of ˜35,000 cells per cm² as shown on the following table.

| | Cells per |
|---|---|
| Vessel | well |
| 3.5 cm | 350,000 |
| Dish | |
| 6-well | 350,000 |
| Plate | |
| 6 cm | 1,000,000 |
| Dish | |
| 10 cm | 2,700,000 |
| Dish | |
| 24 well | 75,000 |
| Plate | |

Cells were allowed to attach overnight. Media was removed, the cells were gently washed to remove cellular debris and a second collagen hydrogel was applied to the top of the cells. The second hydrogel was allowed to set at 30 minutes at 37° C. in 5% CO₂ atmosphere. Doxycycline (Clontech) is added to MSC Growth media to final concentration 25 ng/mL. Doxycycline-containing MSC Growth media (Dox-Growth Media) was added and the cells were incubated for 24 hours.

### Hepatic Maturation

Following 24 hours of incubation in Dox-Growth Media, cells were induced to differentiate to a mature hepatocyte cell type using the following cytokine maturation protocol. Cells were incubated in serum-free Dox+DMEM containing 20 ng/ml of Epidermal Growth Factor and 10 ng/ml of basic Fibroblast Growth Factor (Anti-Pro/DMEM) for 48 hours. The Anti-Pro/DMEM was removed and the cells were liberally washed with PBS. The cells were then cultured with serum-free Dox+DMEM containing 20 ng/ml of Hepatocyte Growth Factor; 10 ηg/ml of basic Fibroblast Growth Factor; and 4.9 mM of Nicotinamide (Diff/DMEM) for 72 hours. Following Diff/DMEM incubation, the cells were washed liberally with PBS and maintained in culture for the remainder of the culture period in Dox+Hepatocyte Culture Media (Lonza, St. Louis, Mo.) containing 20 ng/ml Oncostatin M (Polar/HGM).

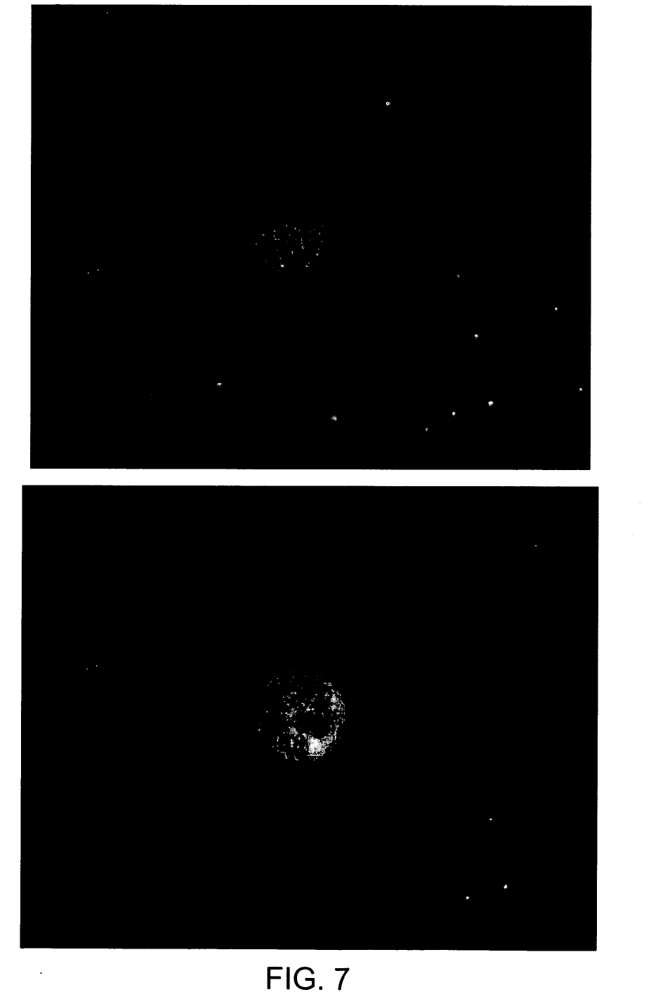

ADMSCs that have been transduced and matured in culture are evaluated for hepatocyte markers using any of the protocols delineated herein or described below. In one embodiment, the presence HNF-4α and Cyp-3A4 is assayed by immunocytochemistry (FIGS. 4, 5, 6A and 6B, 7, and 8).

### Hepatocyte Nuclear Factor-4-α (HNF-4-α)-Staining (H-171)

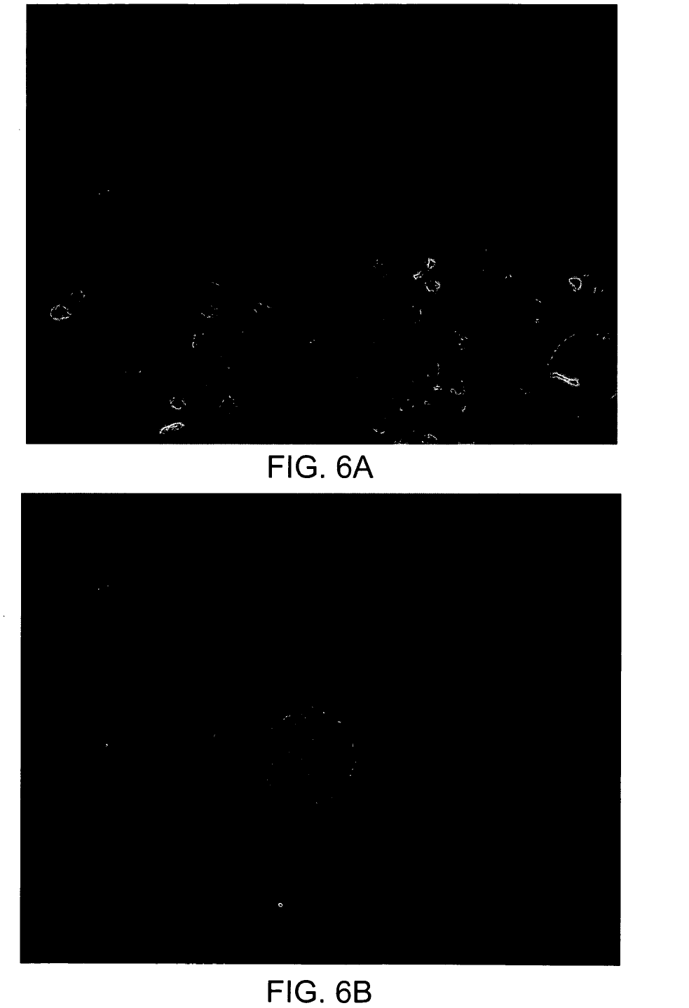

Cells (e.g. HepG2) were fixed for 15 minutes at 24° C. in 4% (w/v) paraformaldehyde in PBS, washed several times with PBS to remove residual paraformaldehyde, and blocked for 30 minutes with PBST intracellular blocking buffer (PBS with 0.1% (v/v) TritonX-100). The fixed cells were then washed several times with PBS and stained with primary antibody (diluted in PBST at 1:50, 1:250, 1:500. Staining was carried out for two hours at 24° C. This can also be done for 24 hrs at 4° C. The cells were washed several times with PBS to remove unattached primary antibody. Cells were stained with secondary antibody (e.g., AlexaFluor 488 goat anti-rabbit (green) or AlexaFluor 523 donkey anti-rabbit (red) (dilute in PBST) at 1:400 for 1 hour at 24° C. Cells were then wash several times with PBS to remove unattached secondary antibody and with DAPI for 10 minutes. Exemplary HNF-4-αstaining in HepG2 cells is shown in FIG. 4. Exemplary staining in primary hepatocytes is shown in FIGS. 6A and 6B.

Cytochrome p450 3A4 (CYP-3A4) Staining

Cells were fixed for 15 minutes at 24° C. in 4% (w/v) PFA in PBS, 15 minutes and washed several times with PBS to remove residual PFA, then blocked for 30 minutes with PBST intracellular blocking buffer (PBS with 0.1% (v/v) TritonX-100). Excess block was removed by washing several times with PBS. The cells were then stained with primary antibody diluted in PBST) at 1:50, 1:250, or 1:500 for 2 hours at 24° C. (can also be stained 24 hrs at 4° C.), then washed several times with PBS to remove unattached primary antibody. Secondary antibody, AlexaFluor 488 goat anti-rabbit (green), was diluted in PBST at 1:400 and cells were stained for 1 hour at 24° C. Unattached secondary antibody was removed by washing several times with PBS to remove. Cells were then stained with DAPI for 10 minutes.

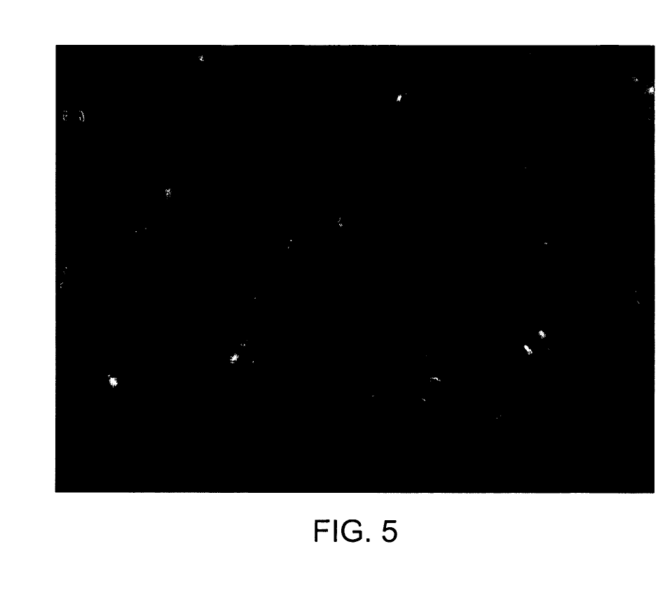

Positive staining was observed at 1:50, 1:250, and 1:500 dilutions. The brightest staining was observed at the 1:50 dilution. The staining appeared cytoplasmic. Some cells expressed CYP-3A4 more strongly than others (FIG. 5).

### CYP-3A4 and HNF-4-α Staining

Two sources of human hepatocytes were stained for Cyp3A4 and HNF-4-α. A cytospun fraction from human liver resection and a tissue section from a human liver resection were stained for CYP-3A4 using 100 μL antibody solution or 250 μL antibody solution. For HNF-4-α, ˜250 μL antibody solution was used. Staining was carried out using the protocol described above except that the secondary antibody was placed on the samples for 2 hours instead of 1 hour. Staining is shown in FIG. 6. The CYP-3A4 antibody appeared to localize throughout the cytoplasm. Every cell appeared to be labeled with antibody

Approximately 90% of primary hepatocytes that labeled with DAPI also stained positively for HNF-4-α. The antibody appeared to localize in two different ways in this cell population. 80% of cells showed cytoplasm staining, and 10% of cells showed nuclear staining

### HNF-4-α Staining on Transfected 3T3 Murine Fibroblasts

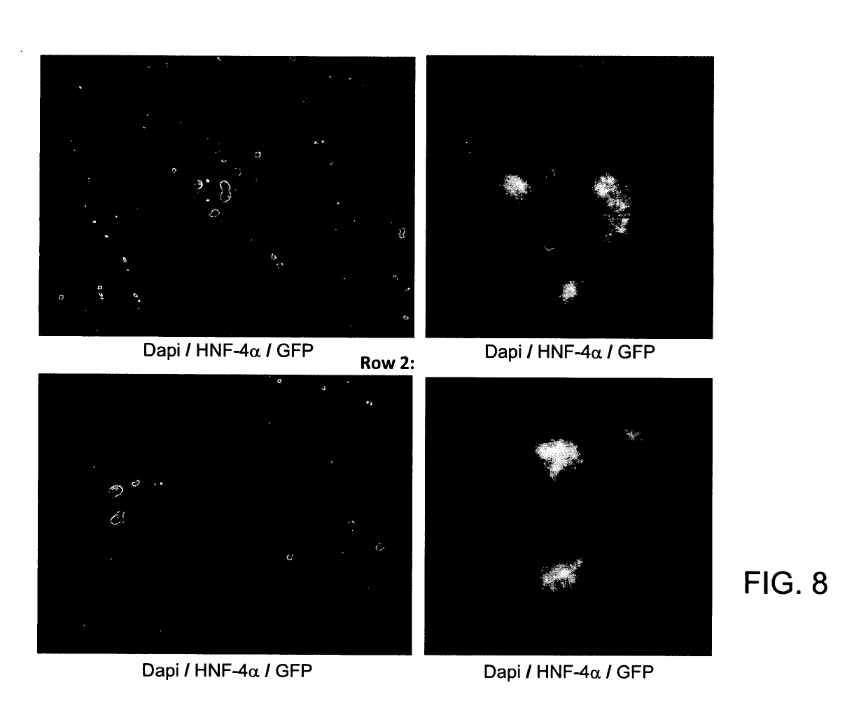

Mouse 3T3 fibroblasts were stained for HNF-4-α (as described above) using a 1:50 dilution of the primary antibody, and the red fluorescent secondary antibody. In the upper panels of FIG. 8, GFP was distributed throughout the cell body. HNF-4-αwas co-localized to the nucleus of the GFP-labeled cells in the lower panels of FIG. 8.

### HNF4α Expression in Transduced ADMSC's is Assayed as Follows:

HNF4α gene expression is compared between transduced cells induced with doxycycline C4P4T1P8 (cells cultured through media process with Doxy), and −Dox C4P4T1P8 (cells cultured through media process without Doxy).

Media is removed from plates with transduced ADMSCs, and 1 ml of Trizol is applied for every 3.5 cm² of surface area (i.e., 1 ml per well of 6-well plate). Cells are lysed and/or homogenized by scraping and the homogenate is incubated for 5 minutes at room temperature. 0.1 ml 1-Bromo, 3-Chloro Propane (Sigma Cat #: B9673) is added per 1 ml of TRIzol, and the mixture is shaken vigorously for 15 seconds, incubated at room temperature for 2 minutes then Centrifuged at 12,000 g for 15 minutes at 4 C until the mixture separates into a lower red phenol-chloroform phase, interphase and the colorless upper aqueous phase where the RNA remains. The aqueous phase is removed and the organic phase is reserved for protein/genomic DNA extraction. 0.5 ml of isopropanol is added to the aqueous phase per 1 ml of TRIzol used for the initial homogenization. The isopropanol/aqueous phase is incubated at room temperature for 5-10 minutes then centrifuged at 12,000 g for 8 minutes at 4-25 C. An RNA precipitate (often invisible before centrifugation) forms a white pellet on the tube. The pellet is washed with 1 ml of 75% ethanol per 1 ml TRI Reagent used for the initial homogenization and the RNA is dissolved in DEPC-treated water. RNA yield is evaluated using Nanodrop.

RNA is then used to generate cDNA using Superscript III First Strand Synthesis System for RT-PCR Cat #: 18080-052 Invitrogen. 324 ng of RNA obtained from transduced cells is used (e.g., DL 71 Transduced Cells Taken Down Jul. 7, 2008) is added to 1 μl of Kit Primer (depending on application), 50 μM Oligo (dT)₂₀, 50 ng/μl Random Hexamers, 1 μl mM dNTP mix, which is brought to 10 μl with DEPC-Treated water. The mixture is incubated at 65 C for 5 minutes then Incubated on Ice for 1 minute. The volume of cDNA product that will be required for a single PCR reaction is 2 μl and one reverse transcription reaction volume yields a total volume of 20 μl. A reaction mix without reverse transcriptase for any one exon gene is carried out to clearly eliminate the possibility of genomic DNA contamination. (i.e. SOX2).

| | | | Volume for |
|---|---|---|---|
| | Volume for | Volume for | 25 |
| | 1 Reaction | 5 Reactions | Reactions |
| Component | (μl) | (μl) | (μl) |
| 10X RT Buffer | 2 | 10 | 50 |
| 50 mM MgCl₂ | 2 | 10 | 50 |
| 0.1M DTT | 2 | 10 | 50 |
| DEPC-Water | 2 | 10 | 50 |
| RNase Out | 1 | 5 | 25 |
| (40 U/ml) | | | |
| Superscript III | 1 | 5 | |
| RT | | | |

10 μl RNA/Primer mix is added to the Synthesis mix. The mixture is typically primed at 25 C for 10 minutes. This step may be skipped if Oligo (dT)₂₀ is used. The mixture is incubated at 50 C for 50 minutes then terminated at 85 C for 5 minutes. cDNA is pelleted by centrifugation and RNA is removed by adding 1 μl of Rnase H then Incubating at 37 C for 20 minutes

PCR Supermix Invitrogen is used to assay gene expression according to commercially available protocols. 10 μl of primer for each gene evaluated is added to Invitogen Platinum PCR supermix. For Each Sample 2 μl of cDNA Product from Superscript cDNA Synthesis is added to 22 μl of Pre-Primer mix. Run on Program HP1

### Protein Expression in HNF4α Transduced ADMSC's.

HNF4α protein expression is compared between transduced ADMSC cells induced with doxycycline C4P4T1P8 (cells cultured through media process with Doxy), −Dox C4P4T1P8 (cells cultured through media process without Doxy), Frozen Human Liver, and Frozen Rat Liver.

Samples are lysed in Lysis Buffer (RIPA buffer) at 4 C. Lysis buffer contains 25 Mm Tris 8.2, 50 mM NaCl, 0.5% DeoxyColas (SigmaD_{—}5670), 0.1% SDS, 0.1% Na azide and 0.5% NP40. PMSF is added to lysis buffer just before use. 200 ul lysis buffer is added to each sample. The samples are homogenized, sonicated (5 s, 2 times, 50% Amplitude on ice), and incubated on ice for 20 minutes, then frozen overnight at −20 C. The next morning the lysates are twice centrifuged at 13,000 rpm, 20 minutes at 4 C. The supernatant is collected and protein concentration is measured.

Protein expression is then analyzed by electrophoresis. 20 ug protein is run on a Biorad precast mini-gel in running buffer (Tris/glycine/SDS, Biorad #161-0732) at 150 mV for 30-40 min, RT (or 100 mV constant voltage, for ˜60 min) Proteins are transferred to PVDF membrane with at 100 mV, 1 hour at room temperature or at 30 mV overnight at 4 C The membrane is incubated in blocking buffer (5% non-fat dry milk in PBS with 0.1% Tween20) for 1 hr at RT. Primary antibodies (α-sarcomeric actin (Sigma, A2172, mouse IgM, 1:500, β-tubulin (Chemicon, MAB3408, mouse IgG2b, 1:2000)) are diluted in blocking buffer and then Incubate overnight at 4 C. The membrane is then rinsed with wash buffer (PBS with 0.1% Tween20), 3×5 minutes, then incubated with secondary antibody (Rabbit anti-Mouse immunoglobin-Ig HRP 1:1000 (DAKO, P0161) or HRP goat anti-mouse IgM HRP 1:1000 (Invitrogen, 62-6820) in blocking buffer at RT for 1-2 hrs. The membrane is then rinsed with wash buffer (PBS with 0.1% Tween20) 3×5 minutes. Protein expression is analysed using a commercially available enhanced chemiluminescence kit.

### Urea Assay

Urea production is one hepatocyte marker. Urea production is assayed in transduced cells induced with doxycycline C4P4T1P8 (cells cultured through media process with Doxy), and −Dox C4P4T1P8 (cells cultured through media process without Doxy) after the cells are incubated in ammonium chloride containing media. 5 mM ammonium-chloride containing medium (5 mM NH₄CL HGM) is made by adding 13.3725 mg of NH₄CL (FW: 53.49 g/mol) to cell growth media. 1 μl of 1000× dox per ml of NH₄CL HGM is added. 500 μl of NH₄CL hepatocyte growth media (HGM) is added to the culture and incubated for 4 hours. Three +dox and three −dox transwells are treated with NH₄CL HGM media. The remaining transwells are treated the sane way, but incubated with just HGM (on OSM; NH₄CL).

In a Clear bottom 96-well plates (e.g. Corning Costar) and plate reader a serial dilution of standard is made. Urea is assayed at an optical density at 430 nm. Urea concentration (mg/dL) of the sample is calculated as

$$[ Urea ] = \frac{( ODSample - ODBlank )}{( ODStandard - ODBlank )} xNx [ S T D ] ( mg \text{/} dl )$$

### Fluorescein-Diacetate Assay

Fluorescein-Diacetate staining is indicative of biliary secretion, which correlates with hepatocyte function. A 1000× Fluorescein-Diacetate solution is made by dissolving 0.010 g of Fluorescein-Diacetate in 1 ml of DMSO to give a 10 mg/ml concentration. HNF4α expression is induced in transduced ADMSC by incubating cells in 10 ml of HGM+Doxycycline. 5 μl of 1000× Fluorescein-Diacetate is added to the culture. The cells are incubated at 37° C. for 35 minutes with the fluorescein-diacetate. The culture is then rinsed three times with PBS at room temperature, and imaged for fluorescence.

### Example 2

### HNF-4-α Expression Reprograms ADSMC

As described herein, ADSMC cells were transfected with a plasmid that induces transient over-production of HNF-4-α. These cells showed production of a highly liver-specific enzyme responsible for drug transformation: cytochrome p450 isozyme CYP3A4. Similar methods can be used for the reprogramming of a number of other cell types. Such cells are listed in the following table:

### Re-Programming Matrix of Factors, ECM and Cytokines

| Differentiated | Transcription | Extra-Cellular | |
|---|---|---|---|
| Cell Type | Factors | Matrix | Cytokine Signals |
| Hepatocytes | 1) HNF4-α | 1) Type I Collagen | 4) EGF, bFGF, |
| | 2) HNF1-α | 2) Type IV Collagen | 5) bFGF, HGF, |
| | 3) HNF3-β | 3) Laminin | Nicotinamide |
| | 4) CEBP/α | | 6) OSM |
| Biliary | 1) HNF 1-B | 1) Type I Collagen | 3) EGF |
| Epithelial | 2) HNF6 | 2) Laminin | 4) bFGF, HGF |
| Cells | | | 5) Dexamethasone |
| Proximal | 1) Pax2 | 1) Type I Collagen | 1) Insulin |
| Tubule | 2) Lhx1 | 2) Type III Collagen | 2) Hydrocortisone |
| Epithelial | 3) HNF1-β | 3) PTFE | 3) BIO |
| Cells* | 4) CEBP/δ | | 4) TGF-β1 |
| | | | 5) EGF |
| | | | 6) Retinoic Acid |

In particular, the invention provides a method for inducing a pluripotent, normally proliferative cell population such as: embryonic stem cells; mesenchymal stem cells; tissue-specific stem cells; or induced pluripotent stem cells, to differentiate into hepatocytes. This process involves the transfer of plasmids containing one, two, three or four genes that function in the maintenance of a mature hepatocyte: HNF4-α; HNF1-α; HNF3-β (FOXA2); and CEBP/α. Such genes can be introduced through transfection or transduction into a pluripotent cell source. These cells are then cultured in a series of supplemented media according to the time course shown below:

| Culture | | | Specific |
|---|---|---|---|
| Time | Medium | Normal Supplements | Supplements |
| 1 to 4 days | DMEM | 10% FBS, Glutamine, | EGF, bFGF |
| | | Pen/Strep, | |
| | | Ampho-B, Gentamicin | |
| 1 to 14 days | DMEM | 10% FBS, Glutamine, | bFGF, HGF, |
| | | Pen/Strep, | Nicotinamide |
| | | Ampho-B, Gentamicin | |
| 1 to 180 days | HGM | | OSM |

A transduced cell of the invention (e.g., a cell delineated herein) comprises any one or more of HNF4-α; HNF1-α; HNF3-β (FOXA2); and CEBP/α. The presence of one or more of the following indicates that the cells have been reprogrammed and express a hepatocyte-like phenotype: Glycogen Storage—Glucose metabolism; Albumin/a-Fetoprotein Secretion—Protein Synthesis; Ammonia-to-urea Conversion—Urea Cycle; CYP 3A4 Glo-Assay—Xenobiotic detoxification; Biliary Fluorescence Assay—Biliary Secretion.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

Listing of sequences:
NM_000457.3 (SEQ. ID. No. 1)
Homo sapiens hepatocyte nuclear factor 4, alpha (HNF4A), transcript
variant 2, mRNA, isoform b.

| | |
|---|---|
| 1 | gggaggaggc agtgggaggg cggagggcgg gggccttcgg ggtgggcgcc cagggtaggg |
| 61 | caggtggccg cggcgtggag gcagggagaa tgcgactctc caaaaccctc gtcgacatgg |
| 121 | acatggccga ctacagtgct gcactggacc cagcctacac caccctggaa tttgagaatg |
| 181 | tgcaggtgtt gacgatgggc aatgacacgt ccccatcaga aggcaccaac ctcaacgcgc |
| 241 | ccaacagcct gggtgtcagc gccctgtgtg ccatctgcgg ggaccgggcc acgggcaaac |
| 301 | actacggtgc ctcgagctgt gacggctgca agggcttctt ccggaggagc gtgcggaaga |
| 361 | accacatgta ctcctgcaga tttagccggc agtgcgtggt ggacaaagac aagaggaacc |
| 421 | agtgccgcta ctgcaggctc aagaaatgct tccgggctgg catgaagaag gaagccgtcc |
| 481 | agaatgagcg ggaccggatc agcactcgaa ggtcaagcta tgaggacagc agcctgccct |
| 541 | ccatcaatgc gctcctgcag gcggaggtcc tgtcccgaca gatcacctcc cccgtctccg |
| 601 | ggatcaacgg cgacattcgg gcgaagaaga ttgccagcat cgcagatgtg tgtgagtcca |
| 661 | tgaaggagca gctgctggtt ctcgttgagt gggccaagta catcccagct ttctgcgagc |
| 721 | tccccctgga cgaccaggtg gccctgctca gagcccatgc tggcgagcac ctgctgctcg |
| 781 | gagccaccaa gagatccatg gtgttcaagg acgtgctgct cctaggcaat gactacattg |
| 841 | tccctcggca ctgcccggag ctggcggaga tgagccgggt gtccatacgc atccttgacg |
| 901 | agctggtgct gcccttccag gagctgcaga tcgatgacaa tgagtatgcc tacctcaaag |
| 961 | ccatcatctt ctttgaccca gatgccaagg ggctgagcga tccagggaag atcaagcggc |
| 1021 | tgcgttccca ggtgcaggtg agcttggagg actacatcaa cgaccgccag tatgactcgc |
| 1081 | gtggccgctt tggagagctg ctgctgctgc tgcccacctt gcagagcatc acctggcaga |
| 1141 | tgatcgagca gatccagttc atcaagctct tcggcatggc caagattgac aacctgttgc |
| 1201 | aggagatgct gctgggaggg tcccccagcg atgcacccca tgcccaccac cccctgcacc |
| 1261 | ctcacctgat gcaggaacat atgggaacca acgtcatcgt tgccaacaca atgcccactc |
| 1321 | acctcagcaa cggacagatg tgtgagtggc cccgacccag gggacaggca gccacccctg |
| 1381 | agaccccaca gccctcaccg ccaggtggct cagggtctga gccctataag ctcctgccgg |
| 1441 | gagccgtcgc cacaatcgtc aagcccctct ctgccatccc ccagccgacc atcaccaagc |
| 1501 | aggaagttat ctagcaagcc gctggggctt gggggctcca ctggctcccc ccagccccct |
| 1561 | aagagagcac ctggtgatca cgtggtcacg gcaaaggaag acgtgatgcc aggaccagtc |
| 1621 | ccagagcagg aatgggaagg atgaagggcc cgagaacatg gcctaagggc cacatcccac |
| 1681 | tgccaccctt gacgccctgc tctggataac aagactttga cttggggaga cctctactgc |
| 1741 | cttggacaac ttttctcatg ttgaagccac tgccttcacc ttcaccttca tccatgtcca |
| 1801 | acccccgact tcatcccaaa ggacagccgc ctggagatga cttgaggcct tacttaaacc |
| 1861 | cagctccctt cttccctagc ctggtgcttc tcctctccta gcccctgtca tggtgtccag |
| 1921 | acagagccct gtgaggctgg gtccaattgt ggcacttggg gcaccttgct cctccttctg |
| 1981 | ctgctgcccc cacctctgct gcctccctct gctgtcacct tgctcagcca tcccgtcttc |
| 2041 | tccaacacca cctctccaga ggccaaggag gccttggaaa cgattccccc agtcattctg |
| 2101 | ggaacatgtt gtaagcactg actgggacca ggcaccaggc agggtctaga aggctgtggt |
| 2161 | gagggaagac gcctttctcc tccaacccaa cctcatcctc cttcttcagg gacttgggtg |
| 2221 | ggtacttggg tgaggatccc tgaaggcctt caacccgaga aaacaaaccc aggttggcga |
| 2281 | ctgcaacagg aacttggagt ggagaggaaa agcatcagaa agaggcagac catccaccag |
| 2341 | gcctttgaga aagggtagaa ttctggctgg tagagcaggt gagatgggac attccaaaga |
| 2401 | acagcctgag ccaaggccta gtggtagtaa gaatctagca agaattgagg aagaatggtg |
| 2461 | tgggagaggg atgatgaaga gagagagggc ctgctggaga gcatagggtc tggaacacca |
| 2521 | ggctgaggtc ctgatcagct tcaaggagta tgcagggagc tgggcttcca gaaaatgaac |
| 2581 | acagcagttc tgcagaggac gggaggctgg aagctgggag gtcaggtggg gtggatgata |
| 2641 | taatgcgggt gagagtaatg aggcttgggg ctggagagga caagatgggt aaaccctcac |
| 2701 | atcagagtga catccaggag gaataagctc ccagggcctg tctcaagctc ttccttactc |
| 2761 | ccaggcactg tcttaaggca tctgacatgc atcatctcat ttaatcctcc cttcctccct |
| 2821 | attaacctag agattgtttt tgttttttat tctcctcctc cctccccgcc ctcacccgcc |
| 2881 | ccactccctc ctaacctaga gattgttaca gaagctgaaa ttgcgttcta agaggtgaag |
| 2941 | tgattttttt tctgaaactc acacaactag gaagtggctg agtcaggact tgaacccagg |
| 3001 | tctccctgga tcagaacagg agctcttaac tacagtggct gaatagcttc tccaaaggct |
| 3061 | ccctgtgttc tcaccgtgat caagttgagg ggcttccggc tcccttctac agcctcagaa |
| 3121 | accagactcg ttcttctggg aaccctgccc actcccagga ccaagattgg cctgaggctg |
| 3181 | cactaaaatt cacttagggt cgagcatcct gtttgctgat aaatattaag gagaattca |

//
NM_001030003.1 (SEQ. ID. No. 2)
Homo sapiens hepatocyte nuclear factor 4, alpha (HNF4A), transcript
variant 5, mRNA, isoform e.

| | |
|---|---|
| 1 | ggccatggtc agcgtgaacg cgcccctcgg ggctccagtg gagagttctt acgacacgtc |
| 61 | cccatcagaa ggcaccaacc tcaacgcgcc caacagcctg ggtgtcagcg ccctgtgtgc |
| 121 | catctgcggg gaccgggcca cgggcaaaca ctacggtgcc tcgagctgtg acggctgcaa |
| 181 | gggcttcttc cggaggagcg tgcggaagaa ccacatgtac tcctgcagat ttagccggca |
| 241 | gtgcgtggtg gacaaagaca agaggaacca gtgccgctac tgcaggctca agaaatgctt |
| 301 | ccgggctggc atgaagaagg aagccgtcca gaatgagcgg gaccggatca gcactcgaag |
| 361 | gtcaagctat gaggacagca gcctgccctc catcaatgcg ctcctgcagg cggaggtcct |
| 421 | gtcccgacag atcacctccc ccgtctccgg gatcaacggc gacattcggg cgaagaagat |
| 481 | tgccagcatc gcagatgtgt gtgagtccat gaaggagcag ctgctggttc tcgttgagtg |
| 541 | ggccaagtac atcccagctt tctgcgagct ccccctggac gaccaggtgg ccctgctcag |
| 601 | agcccatgct ggcgagcacc tgctgctcgg agccaccaag agatccatgg tgttcaagga |
| 661 | cgtgctgctc ctaggcaatg actacattgt ccctcggcac tgcccggagc tggcggagat |
| 721 | gagccgggtg tccatacgca tccttgacga gctggtgctg cccttccagg agctgcagat |
| 781 | cgatgacaat gagtatgcct acctcaaagc catcatcttc tttgacccag atgccaaggg |
| 841 | gctgagcgat ccagggaaga tcaagcggct gcgttcccag gtgcaggtga gcttggagga |
| 901 | ctacatcaac gaccgccagt atgactcgcg tggccgcttt ggagagctgc tgctgctgct |
| 961 | gcccaccttg cagagcatca cctggcagat gatcgagcag atccagttca tcaagctctt |
| 1021 | cggcatggcc aagattgaca acctgttgca ggagatgctg ctgggagggt cccccagcga |
| 1081 | tgcaccccat gcccaccacc ccctgcaccc tcacctgatg caggaacata tgggaaccaa |
| 1141 | cgtcatcgtt gccaacacaa tgcccactca cctcagcaac ggacagatgt ccacccctga |
| 1201 | gaccccacag ccctcaccgc caggtggctc agggtctgag ccctataagc tcctgccggg |
| 1261 | agccgtcgcc acaatcgtca agcccctctc tgccatcccc cagccgacca tcaccaagca |
| 1321 | ggaagttatc tagcaagcc |

//
NM_001030004.1 (SEQ. ID. No. 3)
Homo sapiens hepatocyte nuclear factor 4, alpha (HNF4A), transcript
variant 6, mRNA, isoform f

| | |
|---|---|
| 1 | ggccatggtc agcgtgaacg cgcccctcgg ggctccagtg gagagttctt acgacacgtc |
| 61 | cccatcagaa ggcaccaacc tcaacgcgcc caacagcctg ggtgtcagcg ccctgtgtgc |
| 121 | catctgcggg gaccgggcca cgggcaaaca ctacggtgcc tcgagctgtg acggctgcaa |
| 181 | gggcttcttc cggaggagcg tgcggaagaa ccacatgtac tcctgcagat ttagccggca |
| 241 | gtgcgtggtg gacaaagaca agaggaacca gtgccgctac tgcaggctca agaaatgctt |
| 301 | ccgggctggc atgaagaagg aagccgtcca gaatgagcgg gaccggatca gcactcgaag |
| 361 | gtcaagctat gaggacagca gcctgccctc catcaatgcg ctcctgcagg cggaggtcct |
| 421 | gtcccgacag atcacctccc ccgtctccgg gatcaacggc gacattcggg cgaagaagat |
| 481 | tgccagcatc gcagatgtgt gtgagtccat gaaggagcag ctgctggttc tcgttgagtg |
| 541 | ggccaagtac atcccagctt tctgcgagct ccccctggac gaccaggtgg ccctgctcag |
| 601 | agcccatgct ggcgagcacc tgctgctcgg agccaccaag agatccatgg tgttcaagga |
| 661 | cgtgctgctc ctaggcaatg actacattgt ccctcggcac tgcccggagc tggcggagat |
| 721 | gagccgggtg tccatacgca tccttgacga gctggtgctg cccttccagg agctgcagat |
| 781 | cgatgacaat gagtatgcct acctcaaagc catcatcttc tttgacccag atgccaaggg |
| 841 | gctgagcgat ccagggaaga tcaagcggct gcgttcccag gtgcaggtga gcttggagga |
| 901 | ctacatcaac gaccgccagt atgactcgcg tggccgcttt ggagagctgc tgctgctgct |
| 961 | gcccaccttg cagagcatca cctggcagat gatcgagcag atccagttca tcaagctctt |
| 1021 | cggcatggcc aagattgaca acctgttgca ggagatgctg ctgggaggtc cgtgccaagc |
| 1081 | ccaggagggg cggggttgga gtggggactc cccaggagac aggcctcaca cagtgagctc |
| 1141 | acccctcagc tccttggctt ccccactgtg ccgctttggg caagttgctt aa |

//
NM_175914.3 (SEQ. ID. No. 4)
Homo sapiens hepatocyte nuclear factor 4, alpha (HNF4A), transcript
variant 4, mRNA, isoform d

| | |
|---|---|
| 1 | ggccatggtc agcgtgaacg cgcccctcgg ggctccagtg gagagttctt acgacacgtc |
| 61 | cccatcagaa ggcaccaacc tcaacgcgcc caacagcctg ggtgtcagcg ccctgtgtgc |
| 121 | catctgcggg gaccgggcca cgggcaaaca ctacggtgcc tcgagctgtg acggctgcaa |
| 181 | gggcttcttc cggaggagcg tgcggaagaa ccacatgtac tcctgcagat ttagccggca |
| 241 | gtgcgtggtg gacaaagaca agaggaacca gtgccgctac tgcaggctca agaaatgctt |
| 301 | ccgggctggc atgaagaagg aagccgtcca gaatgagcgg gaccggatca gcactcgaag |
| 361 | gtcaagctat gaggacagca gcctgccctc catcaatgcg ctcctgcagg cggaggtcct |
| 421 | gtcccgacag atcacctccc ccgtctccgg gatcaacggc gacattcggg cgaagaagat |
| 481 | tgccagcatc gcagatgtgt gtgagtccat gaaggagcag ctgctggttc tcgttgagtg |
| 541 | ggccaagtac atcccagctt tctgcgagct ccccctggac gaccaggtgg ccctgctcag |
| 601 | agcccatgct ggcgagcacc tgctgctcgg agccaccaag agatccatgg tgttcaagga |
| 661 | cgtgctgctc ctaggcaatg actacattgt ccctcggcac tgcccggagc tggcggagat |
| 721 | gagccgggtg tccatacgca tccttgacga gctggtgctg cccttccagg agctgcagat |
| 781 | cgatgacaat gagtatgcct acctcaaagc catcatcttc tttgacccag atgccaaggg |
| 841 | gctgagcgat ccagggaaga tcaagcggct gcgttcccag gtgcaggtga gcttggagga |
| 901 | ctacatcaac gaccgccagt atgactcgcg tggccgcttt ggagagctgc tgctgctgct |
| 961 | gcccaccttg cagagcatca cctggcagat gatcgagcag atccagttca tcaagctctt |
| 1021 | cggcatggcc aagattgaca acctgttgca ggagatgctg ctgggagggt cccccagcga |
| 1081 | tgcaccccat gcccaccacc ccctgcaccc tcacctgatg caggaacata tgggaaccaa |
| 1141 | cgtcatcgtt gccaacacaa tgcccactca cctcagcaac ggacagatgt gtgagtggcc |
| 1201 | ccgacccagg ggacaggcag ccacccctga gaccccacag ccctcaccgc caggtggctc |
| 1261 | agggtctgag ccctataagc tcctgccggg agccgtcgcc acaatcgtca agcccctctc |
| 1321 | tgccatcccc cagccgacca tcaccaagca ggaagttatc tagcaagcc |

//
NM_178849.1 (SEQ. ID. No. 5)
Homo sapiens hepatocyte nuclear factor 4, alpha (HNF4A), transcript
variant 1, mRNA, isoform a.

| | |
|---|---|
| 1 | gggaggaggc agtgggaggg cggagggcgg gggccttcgg ggtgggcgcc cagggtaggg |
| 61 | caggtggccg cggcgtggag gcagggagaa tgcgactctc caaaaccctc gtcgacatgg |
| 121 | acatggccga ctacagtgct gcactggacc cagcctacac caccctggaa tttgagaatg |
| 181 | tgcaggtgtt gacgatgggc aatgacacgt ccccatcaga aggcaccaac ctcaacgcgc |
| 241 | ccaacagcct gggtgtcagc gccctgtgtg ccatctgcgg ggaccgggcc acgggcaaac |
| 301 | actacggtgc ctcgagctgt gacggctgca agggcttctt ccggaggagc gtgcggaaga |
| 361 | accacatgta ctcctgcaga tttagccggc agtgcgtggt ggacaaagac aagaggaacc |
| 421 | agtgccgcta ctgcaggctc aagaaatgct tccgggctgg catgaagaag gaagccgtcc |
| 481 | agaatgagcg ggaccggatc agcactcgaa ggtcaagcta tgaggacagc agcctgccct |
| 541 | ccatcaatgc gctcctgcag gcggaggtcc tgtcccgaca gatcacctcc cccgtctccg |
| 601 | ggatcaacgg cgacattcgg gcgaagaaga ttgccagcat cgcagatgtg tgtgagtcca |
| 661 | tgaaggagca gctgctggtt ctcgttgagt gggccaagta catcccagct ttctgcgagc |
| 721 | tccccctgga cgaccaggtg gccctgctca gagcccatgc tggcgagcac ctgctgctcg |
| 781 | gagccaccaa gagatccatg gtgttcaagg acgtgctgct cctaggcaat gactacattg |
| 841 | tccctcggca ctgcccggag ctggcggaga tgagccgggt gtccatacgc atccttgacg |
| 901 | agctggtgct gcccttccag gagctgcaga tcgatgacaa tgagtatgcc tacctcaaag |
| 961 | ccatcatctt ctttgaccca gatgccaagg ggctgagcga tccagggaag atcaagcggc |
| 1021 | tgcgttccca ggtgcaggtg agcttggagg actacatcaa cgaccgccag tatgactcgc |
| 1081 | gtggccgctt tggagagctg ctgctgctgc tgcccacctt gcagagcatc acctggcaga |
| 1141 | tgatcgagca gatccagttc atcaagctct tcggcatggc caagattgac aacctgttgc |
| 1201 | aggagatgct gctgggaggg tcccccagcg atgcacccca tgcccaccac cccctgcacc |
| 1261 | ctcacctgat gcaggaacat atgggaacca acgtcatcgt tgccaacaca atgcccactc |
| 1321 | acctcagcaa cggacagatg tccacccctg agaccccaca gccctcaccg ccaggtggct |
| 1381 | cagggtctga gccctataag ctcctgccgg gagccgtcgc cacaatcgtc aagcccctct |
| 1441 | ctgccatccc ccagccgacc atcaccaagc aggaagttat ctagcaagcc gctggggctt |
| 1501 | gggggctcca ctggctcccc ccagccccct aagagagcac ctggtgatca cgtggtcacg |
| 1561 | gcaaaggaag acgtgatgcc aggaccagtc ccagagcagg aatgggaagg atgaagggcc |
| 1621 | cgagaacatg gcctaagggc cacatcccac tgccaccctt gacgccctgc tctggataac |
| 1681 | aagactttga cttggggaga cctctactgc cttggacaac ttttctcatg ttgaagccac |
| 1741 | tgccttcacc ttcaccttca tccatgtcca acccccgact tcatcccaaa ggacagccgc |
| 1801 | ctggagatga cttgaggcct tacttaaacc cagctccctt cttccctagc ctggtgcttc |
| 1861 | tcctctccta gcccctgtca tggtgtccag acagagccct gtgaggctgg gtccaattgt |
| 1921 | ggcacttggg gcaccttgct cctccttctg ctgctgcccc cacctctgct gcctccctct |
| 1981 | gctgtcacct tgctcagcca tcccgtcttc tccaacacca cctctccaga ggccaaggag |
| 2041 | gccttggaaa cgattccccc agtcattctg ggaacatgtt gtaagcactg actgggacca |
| 2101 | ggcaccaggc agggtctaga aggctgtggt gagggaagac gcctttctcc tccaacccaa |
| 2161 | cctcatcctc cttcttcagg gacttgggtg ggtacttggg tgaggatccc tgaaggcctt |
| 2221 | caacccgaga aaacaaaccc aggttggcga ctgcaacagg aacttggagt ggagaggaaa |
| 2281 | agcatcagaa agaggcagac catccaccag gcctttgaga aagggtagaa ttctggctgg |
| 2341 | tagagcaggt gagatgggac attccaaaga acagcctgag ccaaggccta gtggtagtaa |
| 2401 | gaatctagca agaattgagg aagaatggtg tgggagaggg atgatgaaga gagagagggc |
| 2461 | ctgctggaga gcatagggtc tggaacacca ggctgaggtc ctgatcagct tcaaggagta |
| 2521 | tgcagggagc tgggcttcca gaaaatgaac acagcagttc tgcagaggac gggaggctgg |
| 2581 | aagctgggag gtcaggtggg gtggatgata taatgcgggt gagagtaatg aggcttgggg |
| 2641 | ctggagagga caagatgggt aaaccctcac atcagagtga catccaggag gaataagctc |
| 2701 | ccagggcctg tctcaagctc ttccttactc ccaggcactg tcttaaggca tctgacatgc |
| 2761 | atcatctcat ttaatcctcc cttcctccct attaacctag agattgtttt tgttttttat |
| 2821 | tctcctcctc cctccccgcc ctcacccgcc ccactccctc ctaacctaga gattgttaca |
| 2881 | gaagctgaaa ttgcgttcta agaggtgaag tgattttttt tctgaaactc acacaactag |
| 2941 | gaagtggctg agtcaggact tgaacccagg tctccctgga tcagaacagg agctcttaac |
| 3001 | tacagtggct gaatagcttc tccaaaggct ccctgtgttc tcaccgtgat caagttgagg |
| 3061 | ggcttccggc tcccttctac agcctcagaa accagactcg ttcttctggg aaccctgccc |
| 3121 | actcccagga ccaagattgg cctgaggctg cactaaaatt cacttagggt cgagcatcct |
| 3181 | gtttgctgat aaatattaag gagaattca |

//
NM_178850.1 (SEQ. ID. No. 6)
Homo sapiens hepatocyte nuclear factor 4, alpha (HNF4A), transcript
variant 3, mRNA, isoform c.

| | |
|---|---|
| 1 | gggaggaggc agtgggaggg cggagggcgg gggccttcgg ggtgggcgcc cagggtaggg |
| 61 | caggtggccg cggcgtggag gcagggagaa tgcgactctc caaaaccctc gtcgacatgg |
| 121 | acatggccga ctacagtgct gcactggacc cagcctacac caccctggaa tttgagaatg |
| 181 | tgcaggtgtt gacgatgggc aatgacacgt ccccatcaga aggcaccaac ctcaacgcgc |
| 241 | ccaacagcct gggtgtcagc gccctgtgtg ccatctgcgg ggaccgggcc acgggcaaac |
| 301 | actacggtgc ctcgagctgt gacggctgca agggcttctt ccggaggagc gtgcggaaga |
| 361 | accacatgta ctcctgcaga tttagccggc agtgcgtggt ggacaaagac aagaggaacc |
| 421 | agtgccgcta ctgcaggctc aagaaatgct tccgggctgg catgaagaag gaagccgtcc |
| 481 | agaatgagcg ggaccggatc agcactcgaa ggtcaagcta tgaggacagc agcctgccct |
| 541 | ccatcaatgc gctcctgcag gcggaggtcc tgtcccgaca gatcacctcc cccgtctccg |
| 601 | ggatcaacgg cgacattcgg gcgaagaaga ttgccagcat cgcagatgtg tgtgagtcca |
| 661 | tgaaggagca gctgctggtt ctcgttgagt gggccaagta catcccagct ttctgcgagc |
| 721 | tccccctgga cgaccaggtg gccctgctca gagcccatgc tggcgagcac ctgctgctcg |
| 781 | gagccaccaa gagatccatg gtgttcaagg acgtgctgct cctaggcaat gactacattg |
| 841 | tccctcggca ctgcccggag ctggcggaga tgagccgggt gtccatacgc atccttgacg |
| 901 | agctggtgct gcccttccag gagctgcaga tcgatgacaa tgagtatgcc tacctcaaag |
| 961 | ccatcatctt ctttgaccca gatgccaagg ggctgagcga tccagggaag atcaagcggc |
| 1021 | tgcgttccca ggtgcaggtg agcttggagg actacatcaa cgaccgccag tatgactcgc |
| 1081 | gtggccgctt tggagagctg ctgctgctgc tgcccacctt gcagagcatc acctggcaga |
| 1141 | tgatcgagca gatccagttc atcaagctct tcggcatggc caagattgac aacctgttgc |
| 1201 | aggagatgct gctgggaggt ccgtgccaag cccaggaggg gcggggttgg agtggggact |
| 1261 | ccccaggaga caggcctcac acagtgagct cacccctcag ctccttggct tccccactgt |
| 1321 | gccgctttgg gcaagttgct taacctgtct gtgcctcagt ttcctcacca gaaaaatggg |
| 1381 | aacaaggcaa tggtctattt gttcaggcac cgagaaccta gcacgtgcca gtcactgttc |
| 1441 | taagtgctgg caattcagca aagaacaaga tctttgccct cggggaggct gtgtgtgtgt |
| 1501 | gagtatgtat ggatgcgtgg atatctgtgt atatgcccgt atgtgcgtgc atgtgtatat |
| 1561 | aaagcctcac attttatgat tttgaaataa acaggtaata |

//
NM_000545 (SEQ. ID. No. 7)
Homo sapiens HNF1 homeobox A (HNF1A), mRNA.

| | |
|---|---|
| 1 | cgtggccctg tggcagccga gccatggttt ctaaactgag ccagctgcag acggagctcc |
| 61 | tggcggccct gctcgagtca gggctgagca aagaggcact gatccaggca ctgggtgagc |
| 121 | cggggcccta cctcctggct ggagaaggcc ccctggacaa gggggagtcc tgcggcggcg |
| 181 | gtcgagggga gctggctgag ctgcccaatg ggctggggga gactcggggc tccgaggacg |
| 241 | agacggacga cgatggggaa gacttcacgc cacccatcct caaagagctg gagaacctca |
| 301 | gccctgagga ggcggcccac cagaaagccg tggtggagac ccttctgcag gaggacccgt |
| 361 | ggcgtgtggc gaagatggtc aagtcctacc tgcagcagca caacatccca cagcgggagg |
| 421 | tggtcgatac cactggcctc aaccagtccc acctgtccca acacctcaac aagggcactc |
| 481 | ccatgaagac gcagaagcgg gccgccctgt acacctggta cgtccgcaag cagcgagagg |
| 541 | tggcgcagca gttcacccat gcagggcagg gagggctgat tgaagagccc acaggtgatg |
| 601 | agctaccaac caagaagggg cggaggaacc gtttcaagtg gggcccagca tcccagcaga |
| 661 | tcctgttcca ggcctatgag aggcagaaga accctagcaa ggaggagcga gagacgctag |
| 721 | tggaggagtg caatagggcg gaatgcatcc agagaggggt gtccccatca caggcacagg |
| 781 | ggctgggctc caacctcgtc acggaggtgc gtgtctacaa ctggtttgcc aaccggcgca |
| 841 | aagaagaagc cttccggcac aagctggcca tggacacgta cagcgggccc cccccagggc |
| 901 | caggcccggg acctgcgctg cccgctcaca gctcccctgg cctgcctcca cctgccctct |
| 961 | cccccagtaa ggtccacggt gtgcgctatg gacagcctgc gaccagtgag actgcagaag |
| 1021 | taccctcaag cagcggcggt cccttagtga cagtgtctac acccctccac caagtgtccc |
| 1081 | ccacgggcct ggagcccagc cacagcctgc tgagtacaga agccaagctg gtctcagcag |
| 1141 | ctgggggccc cctcccccct gtcagcaccc tgacagcact gcacagcttg gagcagacat |
| 1201 | ccccaggcct caaccagcag ccccagaacc tcatcatggc ctcacttcct ggggtcatga |
| 1261 | ccatcgggcc tggtgagcct gcctccctgg gtcctacgtt caccaacaca ggtgcctcca |
| 1321 | ccctggtcat cggcctggcc tccacgcagg cacagagtgt gccggtcatc aacagcatgg |
| 1381 | gcagcagcct gaccaccctg cagcccgtcc agttctccca gccgctgcac ccctcctacc |
| 1441 | agcagccgct catgccacct gtgcagagcc atgtgaccca gagccccttc atggccacca |
| 1501 | tggctcagct gcagagcccc cacgccctct acagccacaa gcccgaggtg gcccagtaca |
| 1561 | cccacacggg cctgctcccg cagactatgc tcatcaccga caccaccaac ctgagcgccc |
| 1621 | tggccagcct cacgcccacc aagcaggtct tcacctcaga cactgaggcc tccagtgagt |
| 1681 | ccgggcttca cacgccggca tctcaggcca ccaccctcca cgtccccagc caggaccctg |
| 1741 | ccagcatcca gcacctgcag ccggcccacc ggctcagcgc cagccccaca gtgtcctcca |
| 1801 | gcagcctggt gctgtaccag agctcagact ccagcaatgg ccagagccac ctgctgccat |
| 1861 | ccaaccacag cgtcatcgag accttcatct ccacccagat ggcctcttcc tcccagtaac |
| 1921 | cacggcacct gggccctggg gcctgtactg cctgcttggg gggtgatgag ggcagcagcc |
| 1981 | agccctgcct ggaggacctg agcctgccga gcaaccgtgg cccttcctgg acagctgtgc |
| 2041 | ctcgctcccc actctgctct gatgcatcag aaagggaggg ctctgaggcg ccccaacccg |
| 2101 | tggaggctgc tcggggtgca caggaggggg tcgtggagag ctaggagcaa agcctgttca |
| 2161 | tggcagatgt aggagggact gtcgctgctt cgtgggatac agtcttctta cttggaactg |
| 2221 | aagggggcgg cctatgactt gggcaccccc agcctgggcc tatggagagc cctgggaccg |
| 2281 | ctacaccact ctggcagcca cacttctcag gacacaggcc tgtgtagctg tgacctgctg |
| 2341 | agctctgaga ggccctggat cagcgtggcc ttgttctgtc accaatgtac ccaccgggcc |
| 2401 | actccttcct gccccaactc cttccagcta gtgacccaca tgccatttgt actgacccca |
| 2461 | tcacctactc acacaggcat ttcctgggtg gctactctgt gccagagcct ggggctctaa |
| 2521 | cgcctgagcc cagggaggcc gaagctaaca gggaaggcag gcagggctct cctggcttcc |
| 2581 | catccccagc gattccctct cccaggcccc atgacctcca gctttcctgt atttgttccc |
| 2641 | aagagcatca tgcctctgag gccagcctgg cctcctgcct ctactgggaa ggctacttcg |
| 2701 | gggctgggaa gtcgtcctta ctcctgtggg agcctcgcaa cccgtgccaa gtccaggtcc |
| 2761 | tggtggggca gctcctctgt ctcgagcgcc ctgcagaccc tgcccttgtt tggggcagga |
| 2821 | gtagctgagc tcacaaggca gcaaggcccg agcagctgag cagggccggg gaactggcca |
| 2881 | agctgaggtg cccaggagaa gaaagaggtg accccagggc acaggagcta cctgtgtgga |
| 2941 | caggactaac actcagaagc ctgggggcct ggctggctga gggcagttcg cagccaccct |
| 3001 | gaggagtctg aggtcctgag cactgccagg agggacaaag gagcctgtga acccaggaca |
| 3061 | agcatggtcc cacatccctg ggcctgctgc tgagaacctg gccttcagtg taccgcgtct |
| 3121 | accctgggat tcaggaaaag gcctggggtg acccggcacc ccctgcagct tgtagccagc |
| 3181 | cggggcgagt ggcacgttta tttaactttt agtaaagtca aggagaaatg cggtggaaaa |
| 3241 | a |

//
NM_021784.4 (SEQ. ID. No. 8)
Homo sapiens forkhead box A2 (FOXA2), transcript variant 1, mRNA,
isoform 1.

| | |
|---|---|
| 1 | cccgcccact tccaactacc gcctccggcc tgcccaggga gagagaggga gtggagccca |
| 61 | gggagaggga gcgcgagaga gggagggagg aggggacggt gctttggctg actttttttt |
| 121 | aaaagagggt gggggtgggg ggtgattgct ggtcgtttgt tgtggctgtt aaattttaaa |
| 181 | ctgccatgca ctcggcttcc agtatgctgg gagcggtgaa gatggaaggg cacgagccgt |
| 241 | ccgactggag cagctactat gcagagcccg agggctactc ctccgtgagc aacatgaacg |
| 301 | ccggcctggg gatgaacggc atgaacacgt acatgagcat gtcggcggcc gccatgggca |
| 361 | gcggctcggg caacatgagc gcgggctcca tgaacatgtc gtcgtacgtg ggcgctggca |
| 421 | tgagcccgtc cctggcgggg atgtcccccg gcgcgggcgc catggcgggc atgggcggct |
| 481 | cggccggggc ggccggcgtg gcgggcatgg ggccgcactt gagtcccagc ctgagcccgc |
| 541 | tcggggggca ggcggccggg gccatgggcg gcctggcccc ctacgccaac atgaactcca |
| 601 | tgagccccat gtacgggcag gcgggcctga gccgcgcccg cgaccccaag acctacaggc |
| 661 | gcagctacac gcacgcaaag ccgccctact cgtacatctc gctcatcacc atggccatcc |
| 721 | agcagagccc caacaagatg ctgacgctga gcgagatcta ccagtggatc atggacctct |
| 781 | tccccttcta ccggcagaac cagcagcgct ggcagaactc catccgccac tcgctctcct |
| 841 | tcaacgactg tttcctgaag gtgccccgct cgcccgacaa gcccggcaag ggctccttct |
| 901 | ggaccctgca ccctgactcg ggcaacatgt tcgagaacgg ctgctacctg cgccgccaga |
| 961 | agcgcttcaa gtgcgagaag cagctggcgc tgaaggaggc cgcaggcgcc gccggcagcg |
| 1021 | gcaagaaggc ggccgccgga gcccaggcct cacaggctca actcggggag gccgccgggc |
| 1081 | cggcctccga gactccggcg ggcaccgagt cgcctcactc gagcgcctcc ccgtgccagg |
| 1141 | agcacaagcg agggggcctg ggagagctga aggggacgcc ggctgcggcg ctgagccccc |
| 1201 | cagagccggc gccctctccc gggcagcagc agcaggccgc ggcccacctg ctgggcccgc |
| 1261 | cccaccaccc gggcctgccg cctgaggccc acctgaagcc ggaacaccac tacgccttca |
| 1321 | accacccgtt ctccatcaac aacctcatgt cctcggagca gcagcaccac cacagccacc |
| 1381 | accaccacca accccacaaa atggacctca aggcctacga acaggtgatg cactaccccg |
| 1441 | gctacggttc ccccatgcct ggcagcttgg ccatgggccc ggtcacgaac aaaacgggcc |
| 1501 | tggacgcctc gcccctggcc gcagatacct cctactacca gggggtgtac tcccggccca |
| 1561 | ttatgaactc ctcttaagaa gacgacggct tcaggcccgg ctaactctgg caccccggat |
| 1621 | cgaggacaag tgagagagca agtgggggtc gagactttgg ggagacggtg ttgcagagac |
| 1681 | gcaagggaga agaaatccat aacaccccca ccccaacacc cccaagacag cagtcttctt |
| 1741 | cacccgctgc agccgttccg tcccaaacag agggccacac agatacccca cgttctatat |
| 1801 | aaggaggaaa acgggaaaga atataaagtt aaaaaaaagc ctccggtttc cactactgtg |
| 1861 | tagactcctg cttcttcaag cacctgcaga ttctgatttt tttgttgttg ttgttctcct |
| 1921 | ccattgctgt tgttgcaggg aagtcttact taaaaaaaaa aaaaaatttt gtgagtgact |
| 1981 | cggtgtaaaa ccatgtagtt ttaacagaac cagagggttg tactattgtt taaaaacagg |
| 2041 | aaaaaaaata atgtaagggt ctgttgtaaa tgaccaagaa aaagaaaaaa aaagcattcc |
| 2101 | caatcttgac acggtgaaat ccaggtctcg ggtccgatta atttatggtt tctgcgtgct |
| 2161 | ttatttatgg cttataaatg tgtattctgg ctgcaagggc cagagttcca caaatctata |
| 2221 | ttaaagtgtt atacccggtt ttatcccttg aatcttttct tccagatttt tcttttcttt |
| 2281 | acttggctta caaaatatac aggcttggaa attatttcaa gaaggaggga gggataccct |
| 2341 | gtctggttgc aggttgtatt ttattttggc ccagggagtg ttgctgtttt cccaacattt |
| 2401 | tattaataaa attttcagac ataaaaaa |

//
NM_153675.2 (SEQ. ID. No. 9).
Homo sapiens forkhead box A2 (FOXA2), transcript variant 2, mRNA,
isoform 2.

| | |
|---|---|
| 1 | cggccgctgc tagaggggct gcttgcgcca ggcgccggcc gccccactgc gggtccctgg |
| 61 | cggccggtgt ctgaggagtc ggagagccga ggcggccaga ccgtgcgccc cgcgcttctc |
| 121 | ccgaggccgt tccgggtctg aactgtaaca gggaggggcc tcgcaggagc agcagcgggc |
| 181 | gagttaaagt atgctgggag cggtgaagat ggaagggcac gagccgtccg actggagcag |
| 241 | ctactatgca gagcccgagg gctactcctc cgtgagcaac atgaacgccg gcctggggat |
| 301 | gaacggcatg aacacgtaca tgagcatgtc ggcggccgcc atgggcagcg gctcgggcaa |
| 361 | catgagcgcg ggctccatga acatgtcgtc gtacgtgggc gctggcatga gcccgtccct |
| 421 | ggcggggatg tcccccggcg cgggcgccat ggcgggcatg ggcggctcgg ccggggcggc |
| 481 | cggcgtggcg ggcatggggc cgcacttgag tcccagcctg agcccgctcg gggggcaggc |
| 541 | ggccggggcc atgggcggcc tggcccccta cgccaacatg aactccatga gccccatgta |
| 601 | cgggcaggcg ggcctgagcc gcgcccgcga ccccaagacc tacaggcgca gctacacgca |
| 661 | cgcaaagccg ccctactcgt acatctcgct catcaccatg gccatccagc agagccccaa |
| 721 | caagatgctg acgctgagcg agatctacca gtggatcatg gacctcttcc ccttctaccg |
| 781 | gcagaaccag cagcgctggc agaactccat ccgccactcg ctctccttca acgactgttt |
| 841 | cctgaaggtg ccccgctcgc ccgacaagcc cggcaagggc tccttctgga ccctgcaccc |
| 901 | tgactcgggc aacatgttcg agaacggctg ctacctgcgc cgccagaagc gcttcaagtg |
| 961 | cgagaagcag ctggcgctga aggaggccgc aggcgccgcc ggcagcggca agaaggcggc |
| 1021 | cgccggagcc caggcctcac aggctcaact cggggaggcc gccgggccgg cctccgagac |
| 1081 | tccggcgggc accgagtcgc ctcactcgag cgcctccccg tgccaggagc acaagcgagg |
| 1141 | gggcctggga gagctgaagg ggacgccggc tgcggcgctg agccccccag agccggcgcc |
| 1201 | ctctcccggg cagcagcagc aggccgcggc ccacctgctg ggcccgcccc accacccggg |
| 1261 | cctgccgcct gaggcccacc tgaagccgga acaccactac gccttcaacc acccgttctc |
| 1321 | catcaacaac ctcatgtcct cggagcagca gcaccaccac agccaccacc accaccaacc |
| 1381 | ccacaaaatg gacctcaagg cctacgaaca ggtgatgcac taccccggct acggttcccc |
| 1441 | catgcctggc agcttggcca tgggcccggt cacgaacaaa acgggcctgg acgcctcgcc |
| 1501 | cctggccgca gatacctcct actaccaggg ggtgtactcc cggcccatta tgaactcctc |
| 1561 | ttaagaagac gacggcttca ggcccggcta actctggcac cccggatcga ggacaagtga |
| 1621 | gagagcaagt gggggtcgag actttgggga gacggtgttg cagagacgca agggagaaga |
| 1681 | aatccataac acccccaccc caacaccccc aagacagcag tcttcttcac ccgctgcagc |
| 1741 | cgttccgtcc caaacagagg gccacacaga taccccacgt tctatataag gaggaaaacg |
| 1801 | ggaaagaata taaagttaaa aaaaagcctc cggtttccac tactgtgtag actcctgctt |
| 1861 | cttcaagcac ctgcagattc tgattttttt gttgttgttg ttctcctcca ttgctgttgt |
| 1921 | tgcagggaag tcttacttaa aaaaaaaaaa aaattttgtg agtgactcgg tgtaaaacca |
| 1981 | tgtagtttta acagaaccag agggttgtac tattgtttaa aaacaggaaa aaaaataatg |
| 2041 | taagggtctg ttgtaaatga ccaagaaaaa gaaaaaaaaa gcattcccaa tcttgacacg |
| 2101 | gtgaaatcca ggtctcgggt ccgattaatt tatggtttct gcgtgcttta tttatggctt |
| 2161 | ataaatgtgt attctggctg caagggccag agttccacaa atctatatta aagtgttata |
| 2221 | cccggtttta tcccttgaat cttttcttcc agatttttct tttctttact tggcttacaa |
| 2281 | aatatacagg cttggaaatt atttcaagaa ggagggaggg ataccctgtc tggttgcagg |
| 2341 | ttgtatttta ttttggccca gggagtgttg ctgttttccc aacattttat taataaaatt |
| 2401 | ttcagacata aaaaa |

//
NG_012022 (SEQ ID NO. 10)

| | |
|---|---|
| 1 | aaaagaaatg agttgcttaa tattcaaagt tttgggggaa actagaacct caaaagtgca |
| 61 | cttatggaga gacaaggaga gtagggaagc gtatgccaag ctcttattta ctgagcaagt |
| 121 | gtgtcctgca tgccaggaac tggccttggc acaacattct ccctaatcct cccagcagct |
| 181 | ccctacacca cctgcctcct ggggagacag gataagtaat ctcatgggta gagctgggcg |
| 241 | cctgtggata aaaaacacta ggccggacac agtggcttac acctgtaaat cccagcactt |
| 301 | tggaaagccg aggtgggagg atcacttgag ctcaggagtt cgagaccagc ctgggctgcg |
| 361 | tggtgaaacc ccatctatac aaaaaattag ccaggcatgg tggtgcatgc ctgtagtccc |
| 421 | agctacccag gaggccgatg tgggaggatc acttgagcct gggaggcgga ggttgcattg |
| 481 | agctgagatc caaccactgc tctccagcct gggcaacaga gtgagaccct gtctcgaaaa |
| 541 | aaaaaagaag aaagaaaaag aaacactgga ctttgggttg attatgcgtt gagttcatat |
| 601 | gctttacatg tcaagatcta aagcctaaaa tgaatcagta taaaaccgtt tatgaatcca |
| 661 | tttgtaacaa aaagtaacta gtgtttttat tttgttttgt ttgtctagtt ggaggaaggc |
| 721 | ctttgagagt atcttattga atgattgaga cattattagc tataccatat tagatacttg |
| 781 | tacccctagc cagagctcag ctgagcctga aatcatagtt ctggactttt atcttatctg |
| 841 | aattggtcct gtttggagcg accaatctta cacacacatt tcagcttcag ataaagtcag |
| 901 | aaaactacat cagaagggtt agttccgttt ttgctcttca gaatttgatt gatttccaaa |
| 961 | cttacgtact aattctctcg tgggccttgc attaggaagg ttaaaccaaa gtaaaataaa |
| 1021 | acatttctag ggctctgttt gtcttctcaa aatggttagt gctgtgattg actccagaat |
| 1081 | caggcttcat cctggagcat ccaaaactca ggaccaccgt ttcccgggga ctcacgctca |
| 1141 | accccccaca ctgggcctgt gtacaccccc atccacagcc tcaggaagaa ggaccttaaa |
| 1201 | ggtggtcaca gaagaattta caagctgctc ttaggactgg ctttgtctca gttgcacatg |
| 1261 | agccagagcc acttctccca atcaaaggtg atttctcctt tgctcccact tctccagcca |
| 1321 | caagccaact gtctatgaga ctgtcagaca tcagccccct ctcccttacc ctgtgggact |
| 1381 | ctcccatgga aagtgcctcc atcgacccca gggtctcacc ccaactcccc agctaatttc |
| 1441 | ctgctttagg gcttgggcag gacacttcta tctagggccc tagacacccc ctgtcctctg |
| 1501 | agaaccccgc caggcctagc cagagaccta ccacttgccc tcaaaaccat ttcctcctca |
| 1561 | tcccgaggta cacttttagg cttccaagga aaaaccagaa catggtagat ggatcttcgc |
| 1621 | aactgctcca aggtcttccc ctccaaaacc acaaagcagg caggtgagta tcctggaatg |
| 1681 | agtccccagt ggcctcctgg gggcttggag aggggcctcc aatttcctgg gcattatatg |
| 1741 | tgtcaggggg tggtaatccc agccagaagc ctggcattca gcccccaagt cctgacatct |
| 1801 | tgttctaatc taccctgccc gctcattact ggggcaatac aagtctccat cctttcccat |
| 1861 | ctggtcaaca ccccctctcc gtcttggcca gagtagattc tcaggaaccc aaatccgaca |
| 1921 | acgaagctcc cctgctaaaa tctggaggtc aaaagcctgg gagataaggg ttcccccttc |
| 1981 | acctgcagtg gccttggcca tcagcccagc cctgtctgcg gatccagcct tatctaggcc |
| 2041 | ccaccctccc tcaccccact ctccagccgg ccaaccatct cgttctttgt taccagaaca |
| 2101 | catgcactgg caggctctcc acctttgcac ttaccattcc ctcctcctag aagagccttc |
| 2161 | ctcccttatc cttccagcaa attcctgctc aactttcaag gcccagctca aaggtctgct |
| 2221 | cctgcatgaa gccctgtctc accgccggtt cagtccaagc aggctgccat catagaccta |
| 2281 | ccataaacat ttatttctca tggttctgga ggctggatgc ccaaggccaa agccaagctg |
| 2341 | ccagcaccct caggttctgg tgaggggcct cttccctgct tgcagacggc cacctccttt |
| 2401 | ctgtgcatgc ccatgacatt gttcttctga taaggccacc aatcctatca aattaggacc |
| 2461 | ctacccatat gacatcatct cactttaatg acttcctaaa agccttatct ccaaatacag |
| 2521 | tcacatgggg gttaggcctt caacatagga atttggggca gggacacagt tcagtccata |
| 2581 | acacctccgc agacaaatct gatctctgcc tcctccacag cgccaccttg ttcagggcag |
| 2641 | gacttctctt catcacagca catccaccct tgattcccat ttgtgcctcg tgtgcatttg |
| 2701 | atgaatgaat gaagtcccat cccctactcc ttctgtctcc tccttcctcc cagcagcctc |
| 2761 | tgtgtgacaa cttatccgtc tctcccactg tcccagttcc cagaggcagg agccaccctc |
| 2821 | tcatgcatcc ctgggctctc ggcaccctgc acagggcagg cccgggtggg tgaattctgg |
| 2881 | ttcaattgta ggaggacctg tggcccctgg ggttggcgaa ccccgggccg ggagtcccac |
| 2941 | tccttgccat tgtcgccccc acacattcat cagcccccta ttggacaaag tcttattccc |
| 3001 | attactgagg tttcttccag tcctacgggg cagagttccc gatctctggg caggcaacag |
| 3061 | cagctgtgca aaaaaggcca gacatcgcct tcctcccaca actccacagg ccgggaaccg |
| 3121 | tgcatgtaca gtggcgtttc tttgttccta ggtaggaaaa gccattagtt ttagcaccgc |
| 3181 | agaaaaaaac gcaaatcgct cttggtgctt tttgatttcc gtccaggtct ccctccacag |
| 3241 | gtgaatgcta tatgaatggg tcctgcattt ggctcagcaa gcagctctcc ggctcacatc |
| 3301 | gggatattta tttaacgtat ctggttttta ctccctcccg tccaaaagcg tcaagctaaa |
| 3361 | aatatcttgt ttatgagctc tggaccgaaa acgaagcagt tgggctatta atcactggga |
| 3421 | ctatgttgaa taggaacttg attaaagcgc agtgtgcgtt gcccagatga aactgcttct |
| 3481 | ttactgcgat cgtcgtggaa gctcggggct ctccagcggt gtttttagct gtgccccctc |
| 3541 | cggggctcct gggcgggtcg tggcgccttg ccaggcctaa ggccactgtc ggtgaagggg |
| 3601 | gtcttccctc acctgtaggc aagcgcgcct ctagccggga cgcaggcggc gtcaggcctc |
| 3661 | agcgccagtg gcgaggggcg gcgcgggcaa ggacaggaga cacttgaggg ctcccaagac |
| 3721 | tccatctggg cccagctgac ggtccgtagc cgccaccccg gccccggtaa agaatgcgag |
| 3781 | ggacgcacgt ggctgggggt ctcggtggca agctccttcc cccgccgcgg tgagagcatt |
| 3841 | agctgccgca ctcaaggggc cccagggcct ggccgcctcg gtccctagga tcccggggac |
| 3901 | tacagcgccc ggcgaaccac tgggcagagt tctccctgtg cgtgctcgga cctggcagcc |
| 3961 | cggcggcctg gacaccctcg ctcccgccgt tggcgcccac ctgaatgggg aggcggggga |
| 4021 | ggaagcggtg gcttcgggcc tcgagggctg cggcccccgc ctcggaatcc cggccccaga |
| 4081 | gttaagtttg tctcctccct cggtgcgccc ctccccgtgc tcgccccggc gtccagctcc |
| 4141 | gctagtctgg ggggccccgc ggtcccgcag gaaaaaaaat cggcgactcc acgccgggta |
| 4201 | acagcgccgg ccccgcgcgc ctagcaagcc gcgcgctccg acctggagaa gtaattataa |
| 4261 | agaaagtttt ccagcccggg cggatcgacc ggccaggtcc tccaggctcc cgggccgagg |
| 4321 | ccgcctccgc tccccgcggg gtcctagcgc cctgcgcggc gccgcccatg ggaccccggc |
| 4381 | gtcctccgag aggtacctct gcgcggaatc acaggggtag cctggagatc agagctagga |
| 4441 | gacgcagagc caccgcgctc aggacctctc cgcgcgtccc aaacggcccc ccaccggccc |
| 4501 | cagccccgct gctctgcgct gcagcctccc cgggacgcgg gtccgggaca ggcctggttc |
| 4561 | tggctttgaa agagaatccg cgccccagca gctcaagacc aagactcgcc ctccgccccc |
| 4621 | cacccctacc ccgtgcagcc tcgggatact cctgggctcc cggccgtggc tggatacggg |
| 4681 | cgcctagggc aggcaggagg agggggcccc cgctaccgac cacgtgggcg cgggggcgac |
| 4741 | ggccgggccg ggggcggagc ttggagcgag cgccgcggct ctgctgggcg cgctggaggc |
| 4801 | ggtgggcgtt gcgccgcggc ctgcctgggg agcgcggcgc tgtgccgcgc tggttcgccg |
| 4861 | ccccatgccg gccgcgcgct aggacccagc aggcgccgcg ccgccgcagc ccggggacag |
| 4921 | aggccgcctc ggactctagg gggcgacgcg gcctgccggg tataaaagct gggccggcgc |
| 4981 | gggccgggcc attcgcgacc cggaggtgcg cgggcgcggg cgagcagggt ctccgggtgg |
| 5041 | gcggcggcga cgccccgcgc aggctggagg ccgccgaggc tcgccatgcc gggagaactc |
| 5101 | taactccccc atggagtcgg ccgacttcta cgaggcggag ccgcggcccc cgatgagcag |
| 5161 | ccacctgcag agccccccgc acgcgcccag cagcgccgcc ttcggctttc cccggggcgc |
| 5221 | gggccccgcg cagcctcccg ccccacctgc cgccccggag ccgctgggcg gcatctgcga |
| 5281 | gcacgagacg tccatcgaca tcagcgccta catcgacccg gccgccttca acgacgagtt |
| 5341 | cctggccgac ctgttccagc acagccggca gcaggagaag gccaaggcgg ccgtgggccc |
| 5401 | cacgggcggc ggcggcggcg gcgactttga ctacccgggc gcgcccgcgg gccccggcgg |
| 5461 | cgccgtcatg cccgggggag cgcacgggcc cccgcccggc tacggctgcg cggccgccgg |
| 5521 | ctacctggac ggcaggctgg agcccctgta cgagcgcgtc ggggcgccgg cgctgcggcc |
| 5581 | gctggtgatc aagcaggagc cccgcgagga ggatgaagcc aagcagctgg cgctggccgg |
| 5641 | cctcttccct taccagccgc cgccgccgcc gccgccctcg cacccgcacc cgcacccgcc |
| 5701 | gcccgcgcac ctggccgccc cgcacctgca gttccagatc gcgcactgcg gccagaccac |
| 5761 | catgcacctg cagcccggtc accccacgcc gccgcccacg cccgtgccca gcccgcaccc |
| 5821 | cgcgcccgcg ctcggtgccg ccggcctgcc gggccctggc agcgcgctca aggggctggg |
| 5881 | cgccgcgcac cccgacctcc gcgcgagtgg cggcagcggc gcgggcaagg ccaagaagtc |
| 5941 | ggtggacaag aacagcaacg agtaccgggt gcggcgcgag cgcaacaaca tcgcggtgcg |
| 6001 | caagagccgc gacaaggcca agcagcgcaa cgtggagacg cagcagaagg tgctggagct |
| 6061 | gaccagtgac aatgaccgcc tgcgcaagcg ggtggaacag ctgagccgcg aactggacac |
| 6121 | gctgcggggc atcttccgcc agctgccaga gagctccttg gtcaaggcca tgggcaactg |
| 6181 | cgcgtgaggc gcgcggctgt gggaccgccc tgggccagcc tccggcgggg acccagggag |
| 6241 | tggtttgggg tcgccggatc tcgaggcttg cccgagccgt gcgagccagg actaggagat |
| 6301 | tccggtgcct cctgaaagcc tggcctgctc cgcgtgtccc ctcccttcct ctgcgccgga |
| 6361 | cttggtgcgt ctaagatgag ggggccaggc ggtggcttct ccctgcgagg aggggagaat |
| 6421 | tcttggggct gagctgggag cccggcaact ctagtattta ggataacctt gtgccttgga |
| 6481 | aatgcaaact caccgctcca atgcctactg agtaggggga gcaaatcgtg ccttgtcatt |
| 6541 | ttatttggag gtttcctgcc tccttcccga ggctacagca gacccccatg agagaaggag |
| 6601 | gggagcaggc ccgtggcagg aggagggctc agggagctga gatcccgaca agcccgccag |
| 6661 | ccccagccgc tcctccacgc ctgtccttag aaaggggtgg aaacataggg acttggggct |
| 6721 | tggaacctaa ggttgttccc ctagttctac atgaaggtgg agggtctcta gttccacgcc |
| 6781 | tctcccacct ccctccgcac acaccccacc ccagcctgct ataggctggg cttccccttg |
| 6841 | gggcggaact cactgcgatg ggggtcacca ggtgaccagt gggagccccc accccgagtc |
| 6901 | acaccagaaa gctaggtcgt gggtcagctc tgaggatgta tacccctggt gggagaggga |
| 6961 | gacctagaga tctggctgtg gggcgggcat ggggggtgaa gggccactgg gaccctcagc |
| 7021 | cttgtttgta ctgtatgcct tcagcattgc ctaggaacac gaagcacgat cagtccatcc |
| 7081 | cagagggacc ggagttatga caagctttcc aaatattttg ctttatcagc cgatatcaac |
| 7141 | acttgtatct ggcctctgtg ccccagcagt gccttgtgca atgtgaatgt gcgcgtctct |
| 7201 | gctaaaccac cattttattt ggtttttgtt ttgttttggt tttgctcgga tacttgccaa |
| 7261 | aatgagactc tccgtcggca gctgggggaa gggtctgaga ctccctttcc ttttggtttt |
| 7321 | gggattactt ttgatcctgg gggaccaatg aggtgagggg ggttctcctt tgccctcagc |
| 7381 | tttccccagc ccctccggcc tgggctgccc acaaggcttg tcccccagag gccctggctc |
| 7441 | ctggtcggga agggaggtgg cctcccgcca acgcatcact ggggctggga gcagggaagg |
| 7501 | acggcttggt tctcttcttt tggggagaac gtagagtctc actctagatg ttttatgtat |
| 7561 | tatatctata atataaacat atcaaagtca atgtcggtgt ctttttaaaa ccagaaagaa |
| 7621 | gctacttcca aggttgtctg tgggccaggt cacatttgta aataatacag cattttccct |
| 7681 | ggcggcaatc ctgactttca tgagctctcc atccatcctg agcccctctt accctaaggg |
| 7741 | ggtgacttac ttcccccagg caagacaaat aaatagcaga ggacaaggct ccaaatggag |
| 7801 | tatgtccaga gcctgaaggc agtctcttgg ggtcagggga gggggctgaa ggggttactg |
| 7861 | ggctgaggcc ttggcgaggc ttcttatctg ccccggggag gaggagaggg agtcctctgc |
| 7921 | ctgaggggta ggcctggcta agcagcccta ggctcaagga gccctttgtg cagacttcct |
| 7981 | tgcaaatcac ctacagctgc agccctggcc actcacacac accgcagctc cagattccag |
| 8041 | caggaccctc ggccagcagg aagaggcctc cagtggtagg accctccaac cctctcctct |
| 8101 | ttccctagac catgtggcta caccctaccc ctgcgaggcc cgaaggcagc ctgaagagag |
| 8161 | agacccctcc atccagccct ccacacctgc ccagccccag ccctcacaag aaagggggct |
| 8221 | gaggcaggcc tgcaacaggg gtctggtcct gccccacaca ctggctgttc aagaacccac |
| 8281 | ccagcatcca cactccccac tgcattcatc ccagacccag caggggactg agtgctggtg |
| 8341 | tgcccccaac tccaatgagc accctgtaag gtcaggcact ccggcacccc cttagcagca |
| 8401 | gtgacagcca agcaagggag ggcagccctt atccctgaac taacaaggcc cgggagaaag |
| 8461 | aggcccagga ggctgatggt gcttttcaga ttttaccctc acggagggta aaagataaat |
| 8521 | tgcacttttc tttttttttt aactctgtac cgagagaatg ttcactgagt ggcttactgt |
| 8581 | gtgtaccaat gcgggctggg gcattttcga gaaattacac attcctttta cactctggaa |
| 8641 | gcttagcttt acctgcctgc gtggttagct ggcacccctt ccggcccccc actctgtcct |
| 8701 | gtgtctggcc gcctcctcac ccacggccgc ccttggcaga ggagaggcca cgagcccagt |
| 8761 | gaggaggcgg cgcctgcccc gccaccctgg gaacagagga ggcagagccc cgccgcgtgc |
| 8821 | cccgaggcct ggggcaggtc gcagcttgct aagccccagc aaaccctctg agttcagtgc |
| 8881 | taattccagt ttttcttgtt tgagggggct gatccctggc ttcccccttt ccccccagaa |
| 8941 | caggtcctag gacgggaggg ggtggggcga ccctcctgcc ctcactcctt cccctcctct |
| 9001 | ctgctgtgcc agcccgctag gggcttcctc ttttaaagct gatggctccc ggaagggatc |
| 9061 | cctcctaccc cgagtcctcc tcctggtcct gagttccagg gtgggctttg ttagggacaa |
| 9121 | gaaggatcca atcacatcca gccccaggaa agggatgagg agggcgaggc ccccgggggg |
| 9181 | ggccaaagta gaggggagga aggtcttttg ggcggcatgg ggtgaacctg cccagcatgt |
| 9241 | accccgtgtc cagctgtagg ggcttcccgg gaacagagtc acccttggcc acccaccgct |
| 9301 | tcctgcaggg ttcctcacag ttcccctcct gactacagcc ttcaggaaga accattccaa |
| 9361 | gagcccagca ctgaacacac ctcaagtatt tatcagatgc ctactgtatg ccagggcctg |
| 9421 | cgccaaagat ggggcactgg gctgggaaca agccgactct gctctggctc ttggggagct |
| 9481 | cacacccttg aggccctaag ctgtggttta aggttgccaa gtgcaatgaa ggggatctca |
| 9541 | gagtgtgaac agggtccacc agggagggct ttatggaggc agcagctttga |

1. A method for generating a hepatocyte-like cell, the method comprising:
culturing a stem cell in a three dimensional culture;
expressing in the stem cell a heterologous nucleic acid molecule encoding a HNF-4-α polypeptide; and
contacting the stem cell with one or more agents selected from the group consisting of epidermal growth factor, basic fibroblast growth factor, hepatocyte growth factor, nicotinamide, and oncostatin M, thereby generating a hepatocyte-like cell.

2. The method of claim 1, wherein the stem cell is an adipose-derived mesenchymal stem cell (ADMSC), embryonic stem cell, mesenchymal stem cell, tissue-specific stem cell, or induced pluripotent stem cell.

3. A method for generating a hepatocyte-like cell, the method comprising:
culturing a adipose-derived mesenchymal stem cell (ADMSC) in a three dimensional culture;
expressing in the stem cell a heterologous nucleic acid molecule encoding a HNF-4-α polypeptide; and
contacting the stem cell with epidermal growth factor, basic fibroblast growth factor, hepatocyte growth factor, nicotinamide, and oncostatin M, thereby generating a hepatocyte-like cell.

4. The method of claim 1, wherein the stem cell further comprises a heterologous nucleic acid molecule selected from the group consisting of HNF-3β, HNF-1α, and CEBP/α.

5. The method of claim 1, wherein HNF-4-α polypeptide is expressed in a viral vector.

6. The method of claim 1, wherein the hepatocyte-like cell expresses a hepatocyte marker selected from the group consisting of glucose-6-phosphatase, albumin secretion, arginase Type I, CYP 3A4, and bile or exhibits a biological activity selected from the group consisting of glucose metabolism, protein synthesis, urea production, xenobiotic detoxification, biliary secretion.

7. The method of claim 1, wherein the cell expresses HNF-4-α, Cyp-3A4, and Zona Occludens-1.

8. The method of claim 1, wherein the three dimensional culture is a collagen sandwich culture.

9. A hepatocyte-like cell produced by the method of claim 1.

10. A hepatocyte-like cell comprising a heterologous nucleic acid molecule encoding a HNF-4-α polypeptide and expressing a hepatocyte marker or hepatocyte biological activity.

11. A pharmaceutical composition comprising the hepatocyte-like cell of claim 10.

12. A method for treating a subject in need of an increase in liver function, the method comprising administering to the subject a hepatocyte-like cell of claim 9, thereby increasing liver function.

13. A method of characterizing agent toxicity, the method comprising contacting a hepatocyte-like cell produced by the method of claim 1 with an agent and identifying a reduction in the cell's metabolic activity or viability.

14. A method for producing a coagulation factor, the method comprising culturing a hepatocyte-like cell produced by the method of claim 1, and isolating from the cell a coagulation factor.

15. A method of neutralizing a toxic compound in a bodily fluid of a mammal, the method comprising contacting the fluid with a hepatocyte-like cell produced by the method of claim 1, wherein the contacting step takes place in a perfusion device.

16. A bioartificial liver device comprising a hepatocyte-like cell produced by the method of claim 1.

17. A method of treating a subject for reduced liver function, the method comprising administering to the subject the bioartifical liver device of claim 16.
